SEQUENCE LISTING (3 sequences)

SEQ ID NO: 1 - protein, 13 aa, Homo sapiens
  MOD_RES (1)..(1): Acetylation
SYSMEHFRWGKPV

SEQ ID NO: 2 - protein, 50 aa, Homo sapiens
SSRRCVRLHESCLGQQVPCCDPCATCYCRFFNAFCYCRKLGTAMNPCSRT

SEQ ID NO: 3 - protein, 46 aa, Homo sapiens
CVRLHESCLGQQVPCCDPCATCYCRFFNAFCYCRKLGTAMNPCSRT

(19) United States
(12) Patent Application Publication
(10) Pub. No.: US 2009/0076029 A1
(43) Pub. Date: Mar. 19, 2009

(54) Compounds and Methods for Treating Obesity

(75) Inventors: Shubh D. SHARMA, Cranbury, NJ (US); Kevin D. Burris, Indianapolis, IN (US); Ramesh Rajpurohit, Hillsboro, NJ (US)

Correspondence Address: MORGAN LEWIS & BOCKIUS LLP, 1111 PENNSYLVANIA AVENUE NW, WASHINGTON, DC, 20004, US

(73) Assignee: Palatin Technologies, Inc., Cranbury, NJ (US)

(21) Appl. No.: 12/130,316

(22) Filed: May 30, 2008

Related U.S. Application Data
(60) Provisional application No. 60/924,837, filed on Jun. 1, 2007.

(51) Int. Cl. A61K 31/496; G01N 33/53; A61P 3/04

(52) U.S. Cl. 514/254.09; 435/7.1

(57) ABSTRACT

Methods for selection of compounds for treatment of obesity, compounds selected by the disclosed methods, and methods of treatment of obesity, wherein a selective melanocortin-4 receptor compound is identified, which compound is further characterized in that it attenuates the binding of both an agonist, including alpha-melanocyte stimulating hormone, and an inverse agonist, including agouti-related protein, to a melanocortin receptor, including melanocortin-4 receptor.

### CROSS-REFERENCE TO RELATED APPLICATIONS

This application claims priority to U.S. Provisional Patent Application No. 60/924,837 filed Jun. 1, 2007, which is herein incorporated by reference in its entirety.

A related U.S. application entitled Methods for Selection of Melanocortin Receptor-Specific Agents for Treatment of Obesity is being filed concurrently herewith, Attorney Docket No. 056291-5366, and the specification and claims thereof are incorporated herein by reference.

### BACKGROUND OF THE INVENTION

1. Field of the Invention (Technical Field)

The present invention relates to compounds, methods of treatment and methods of selecting compounds for treatment of obesity and other disorders, and in particular, compounds for treatment of obesity and related energy homeostasis or feeding disorders characterized by excess weight gain, wherein the compound attenuates the binding of both alpha-melanocyte stimulating hormone (α-MSH) and agouti-related protein (AgRP) to melanocortin receptors.

2. Description of Related Art

Note that the following discussion refers to a number of publications by author(s) and year of publication, and that due to recent publication dates certain publications are not to be considered as prior art vis-a-vis the present invention. Discussion of such publications herein is given for more complete background and is not to be construed as an admission that such publications are prior art for patentability determination purposes.

A family of melanocortin receptor types and subtypes have been identified, including melanocortin 1 receptors (MC1-R) expressed on normal human melanocytes and melanoma cells, melanocortin 2 receptors (MC2-R) for adrenocorticotropin (ACTH) expressed in cells of the adrenal gland, melanocortin 3 and melanocortin 4 receptors (MC3-R and MC4-R), expressed primarily in cells in the hypothalamus, mid-brain and brainstem, and melanocortin 5 receptors (MC5-R), expressed in a wide distribution of tissues.

It has been hypothesized that compounds specific for MC3-R or MC4-R are useful in regulation of energy homeostasis, including use as agents for attenuating food intake and body weight gain, for use in treatment of anorexia and cachexia, for treatment of obesity, and treatment of other food intake and metabolism-related indications. However, the mechanism of action of compounds specific for MC3-R or MC4-R as agents for regulation of energy homeostasis has not been fully elucidated.

Most MC4-R agonist compounds that have been evaluated for use in attenuating food intake and body weight gain also have other systemic effects relating to the melanocortin system, including inducing or facilitating penile erection in males. These effects render most MC4-R agonists unsuitable for use in treatment of obesity. There is a need for melanocortin-specific agents which are efficacious for treatment of obesity, but without other systemic effects, such as induction of penile erections in males, or a sexual response generally in mammals.

While most reports suggest that MC4-R agonists may be employed for attenuating food intake and body weight gain, it is clear that agouti-related protein (AgRP), an endogenous inverse agonist, plays a critical role in the regulatory system. It has been shown that melanocortin receptors, including MC4-R, can express a basal or constitutive level (i.e., unstimulated) of adenylyl cyclase activity, even in the absence of a specific agonist. This effect is particularly pronounced in high receptor density expression systems. (Nijenhuis, W. A. J., Oosterom J. and Adan, R. A. H. AgRP (83-132) acts as an inverse agonist on the human-melanocortin-4 receptor. Molecular Endocrinology 15(1):164-171, 2001.) Adenylyl cyclase is an enzyme of the lyase class that catalyzes the formation of 3′,5′-cyclic adenosine monophosphate (cAMP) from ATP (adenosine triphosphate, the 5′ triphosphate of adenosine). It is hypothesized that AgRP is an inverse agonist, decreasing the activation of andenylyl cyclase. (Id.; but see Dinulescu, D. M. and Cone, R. D. Agouti and agouti-related protein: analogies and contrasts (minireview). J. Biological Chemistry 275(10):6695-6698, 2000). At least one group has suggested that a neutral antagonist of AgRP may produce agonist-like effects in vivo. (Adan, R. A. H. and Kas, M. J. H. Inverse agonist gains weight. TRENDS in Pharmacological Sciences 24(6):315-321, 2003.) However, no such compounds have heretofore been described. It is further clear that the melanocortin receptor system is complex, involving a variety of regulatory mechanisms in addition to a classical agonist/antagonist system. (See, e.g., Ollmann, M. M. et al. Antagonist of central melanocortin receptors in vitro and in vivo by agouti-related protein. Science 278:135-138, 1997; Yang, Y.-K., et al. Characterization of agouti-related protein binding to melanocortin receptors. Molecular Endocrinology 13:148-155, 1999; Kim, M.-S., et al. Sustained orexigenic effect of agouti related protein may not be mediated by the melanocortin 4 receptor. Peptides 23:1069-1076, 2002; Shinyama, H., Masuzaki, H., Fang, H. and Flier, J. S. Regulation of melanocortin-4 receptor signaling: agonist-mediated desensitization and internalization. Endocrinology 133(4):1301-1314, 2003.) Further, all or virtually all MC4-R agonists compounds reported for attenuating food intake or body weight gain in animal models have shown a “rebound” effect, with animals gaining weight equal to or, in most instances, exceeding controls upon cessation of administration of the compounds. There is thus a need for compounds which attenuate food intake or body weight gain without causing a rebound effect upon cessation of administration of the compound.

U.S. Patent Application Pub. No. 2002/0198152 A1 and U.S. Pat. No. 6,451,783, both by Hadcock and Swick and assigned to Pfizer Inc., disclose a method of treating obesity, comprising administration of a compound that attenuates the binding of AgRP to melanocortin receptors, but does not affect the binding of alpha-melanocyte stimulating hormone (α-MSH, an endogenous regulatory hormone) to melanocortin receptors. However, the methods disclosed therein and compounds selected by such method have not been shown to be efficacious in vivo.

U.S. Pat. No. 5,908,609, by Lee, Huszar and Gu and assigned to Millennium Pharmaceuticals, Inc., disclosed a method for identifying compounds that regulate body weight, including contacting a test compound with a cell which expresses a functional MC4-R, and determining whether the compound activates the receptor. It is claimed that compounds that “activate” MC4-R are identified as compounds for inducing weight loss. However, “activation” is defined in terms of induction of cAMP accumulation, and thus this patent simply claims a method of selecting a MC4-R agonist. Similarly, U.S. Pat. No. 6,716,810, by Brennan and Hochgeschwender and assigned to Eleanor Roosevelt Institute and Oklahoma Medical Research Foundation, disclose methods of regulating body weight, such as by administration of α-MSH, an α-MSH analog, or an α-MSH homolog having agonist activity.

### SUMMARY OF THE INVENTION

In one aspect, the invention provides a method of treating weight related disorders. Such weight related disorders can include obesity, such as obesity based upon a body mass index or other diagnostic tool, or may include above-optimum weight, including self-diagnosed above-optimum weight. The weight related disorder may further be a risk of becoming obese, such as a patient at risk of becoming obese based on genetic considerations, heredity, co-factors, life style, or other factors. The weight related disorder may further include energy homeostasis or feeding disorders characterized by excess weight gain. The weight related disordered may be present in a human or non-human mammal. The method includes administering to an obese patient or patient at risk of becoming obese a therapeutically effective amount of a compound or a pharmaceutically acceptable salt thereof that attenuates the binding of both an agonist as to one or more melanocortin receptors and an inverse agonist as to the same one or more melanocortin receptors. Preferably the melanocortin receptor is melanocortin-4 receptor (MC4-R). The agonist may be alpha-melanocyte stimulating hormone (α-MSH) and the inverse agonist may be agouti-related protein (AgRP), such as wherein α-MSH is NDP-α-MSH and AgRP is AgRP (83-132). Alternatively, the inverse agonist may be agouti signaling peptide.

The compound may be an agonist as to human MC4-R, may be a partial agonist as to human MC4-R, may be functionally inactive or a neutral antagonist as to human MC4-R, may be an antagonist as to human MC4-R or may be an inverse agonist as to human MC4-R. Thus, the compound may be an agonist, antagonist, neutral antagonist, or inverse antagonist in a functional MC4-R cAMP assay. Preferably, the compound is more specific for MC4-R than for any of melanocortin-1 receptor (MC1-R), melanocortin-3 receptor (MC3-R) or melanocortin-5 receptor (MC5-R).

In a further aspect of the invention, the compound has a Ki (nM) at MC4-R, determined with respect to α-MSH, which is at least an order of magnitude lower than the Ki (nM) for any of MC1-R, MC3-R or MC5-R. In yet another further aspect, the compound attenuates the binding of both α-MSH as to MC4-R and AgRP as to MC4-R by inhibiting, in a competitive inhibition assay, at least about 90% of the binding of each α-MSH and AgRP to MC4-R.

In another aspect, the compound is an agonist or partial agonist as to MC4-R and has a Ki (nM) at MC4-R, determined with respect to an MC4-R agonist, that is half or less than half of the EC₅₀ (nM) at MC4-R. The MC4-R agonist may be α-MSH or NDP-α-MSH. The Ki (nM) may be at least one order of magnitude less than the EC₅₀ (nM), and preferably the Ki (nM) may be more than one order of magnitude less than the EC₅₀ (nM).

In another aspect, the compound has an intrinsic activity of less than about 0.1 (10%), based on maximal stimulation of adenylyl cyclase achievable by the compound in a MC4-R cell system where the maximal stimulation achieved by α-MSH or NDP-α-MSH is designated as an intrinsic activity of 1.0 (100%), and has a Ki (nM) at MC4-R, determined with respect to an MC4-R agonist, that is half or less than half of the EC₅₀ (nM) at MC4-R. Here too, the MC4-R agonist may be α-MSH or NDP-α-MSH. The Ki (nM) may be at least one order of magnitude less than the EC₅₀ (nM), and preferably the Ki (nM) is more than one order of magnitude less than the EC₅₀ (nM). The Ki (nM) may further be more than two orders of magnitude less than the EC₅₀ (nM).

In yet another aspect, the compound is a non-selective MC3-R and MC4-R agonist, a non-selective MC3-R and MC4-R antagonist, an agonist for MC3-R and antagonist for MC4-R, an antagonist for MC3-R and agonist for MC4-R, an inverse agonist for MC3-R and agonist for MC4-R, an inverse agonist for MC3-R and antagonist for MC4-R or an agonist for MC3-R and inverse agonist for MC4-R. The compound selected by the method may further be characterized in that the compound does not induce or initiate a sexual response in a mammal.

In another and distinct aspect of the invention, there is provided a method of identifying a compound as a candidate for treating weight related disorders, such as obesity and/or related energy homeostasis or feeding disorders characterized by excess weight gain, the method comprising the steps of:
providing a melanocortin receptor assay system;
determining if the compound attenuates the binding of an agonist to a melanocortin receptor in the melanocortin receptor assay system;
determining if the compound attenuates the binding of an inverse agonist to the same melanocortin receptor in the melanocortin receptor assay system; and
selecting the compound as a candidate compound for said treatment if the compound attenuates the binding of both the agonist and the inverse agonist to the melanocortin receptor in the melanocortin receptor assay system.
   In this method the melanocortin receptor assay system may comprise an MC4-R assay system.

The agonist may be alpha-melanocyte stimulating hormone (α-MSH) and the inverse agonist may be agouti-related protein (AgRP), such as wherein α-MSH is NDP-α-MSH and AgRP is AgRP (83-132).

The method may further comprise the steps of:
determining the Ki (nM) of the compound with respect to MC4-R and at least one other melanocortin receptor; and
selecting the compound If the compound has a Ki (nM) with respect to MC4-R that is at least about ten times lower than the Ki (nM) with respect to the at least one other melanocortin receptor.
   The at least one other melanocortin receptor may comprise MC1-R, MC3-R or MC5-R.

The method may further comprise the steps of:
determining specificity of the compound for MC1-R, MC3-R, MC4-R and MC5-R; and
selecting the compound as a candidate compound for said treatment if the compound is more specific for MC4-R than for MC1-R, MC3-R or MC5-R.

In the practice of the method, selecting the compound may comprise selecting the compound wherein the compound attenuates the binding of both α-MSH as to MC4-R and AgRP as to MC4-R by inhibiting, in a competitive inhibition assay, at least about 90% of the binding of each α-MSH and AgRP to MC4-R. In the method, compounds having a Ki (nM) of less than about 10 as to both α-MSH binding to MC4-R and AgRP binding to MC4-R may be selected, such as compounds having a Ki (nM) of less than about 5 as to both α-MSH binding to MC4-R and AgRP binding to MC4-R. In one method, compounds having a Ki (nM) as to α-MSH binding to MC4-R that is no more than about five times higher or lower than the Ki (nM) as to AgRP binding to MC4-R are selected, such as compounds having a Ki (nM) as to α-MSH binding to MC4-R that is no more than about three times higher or lower than the Ki (nM) as to AgRP binding to MC4-R.

The method may further comprise the steps of:
determining the Ki (nM) of the compound at MC4-R;
determining the EC₅₀ of the compound with respect to MC4-R; and
selecting the compound as a candidate compound for said treatment if the compound has a Ki (nM) value that is half or less than half of the EC₅₀ (nM) value. In this method, the MC4-R agonist may be α-MSH or NDP-α-MSH. Selecting the compound may comprise selecting the compound If the compound has a Ki (nM) value that is at least five times less than the EC₅₀ (nM) value. Alternatively, selecting the compound may comprise selecting the compound If the compound has a Ki (nM) value that is at least ten times less than the EC₅₀ (nM) value. In a further alternative, selecting the compound may comprise selecting the compound that has a Ki (nM) value that is more than at least ten times less than the EC₅₀ (nM) value.

The method may further comprise the steps of:
determining the intrinsic activity of the compound based on maximal stimulation of adenylyl cyclase achievable by the compound in a MC4-R cell system where the maximal stimulation achieved by α-MSH or NDP-α-MSH is designated as an intrinsic activity of 1.0 (100%); and
selecting the compound as a candidate compound for said treatment if it has an intrinsic activity of less than about 0.1 (10%).

Such method may further comprise the steps of:
determining the Ki (nM) of the compound at MC4-R;
determining the EC₅₀ of the compound with respect to MC4-R; and
selecting the compound as a candidate compound for said treatment if the compound has a Ki (nM) value that is half or less than half the EC₅₀ (nM) value. The MC4-R agonist may be α-MSH or NDP-α-MSH. Selecting the compound may comprise selecting the compound If the compound has a Ki (nM) value that is at least five times less than the EC₅₀ (nM) value, at least ten times less than the EC₅₀ (nM) value, more than at least ten times less than the EC₅₀ (nM) value or more than at least one hundred times less than the EC₅₀ (nM) value.

The method may further comprise the steps of:
determining the functional status of the compound as an agonist, partial agonist, antagonist or as inactive in a MC4-R cell system; and
selecting the compound as a candidate compound for said treatment if it is an antagonist.

The method may further comprise the steps of:
determining the functional status of the compound as an agonist, partial agonist, inactive or an antagonist in a MC4-R cell system; and
selecting the compound If it is inactive. Selecting the compound If it is inactive may comprise selecting the compound wherein intrinsic activity of the compound based on maximal stimulation of adenylyl cyclase achievable by the compound in a MC4-R cell system is less than about 0.1 (10%) where the maximal stimulation achieved by α-MSH or NDP-α-MSH is designated as an intrinsic activity of 1.0 (100%).

In another and distinct aspect of the invention, there is provided a method of selecting a compound as a candidate compound for treating weight related disorders such as obesity and/or related energy homeostasis or feeding disorders characterized by excess weight gain, which compound does not induce or initiate a sexual response in a mammal, the method comprising the steps of:
providing a melanocortin receptor assay system;
determining if the compound attenuates the binding of an agonist to a melanocortin receptor in the melanocortin receptor assay system;
determining if the compound attenuates the binding of an inverse agonist to the same melanocortin receptor in the melanocortin receptor assay system;
selecting the compound as a candidate compound for said treatment if the compound attenuates the binding of both the agonist and the inverse agonist to the melanocortin receptor in the melanocortin receptor assay system; and
wherein the compound does not induce or initiate a sexual response in a mammal. In this method the melanocortin receptor assay system may comprise an MC4-R assay system. The agonist may be alpha-melanocyte stimulating hormone (α-MSH) and the inverse agonist may be agouti-related protein (AgRP) such as wherein α-MSH is NDP-α-MSH and AgRP is AgRP (83-132).

The method may further comprise the steps of:
determining the Ki (nM) of the compound with respect to MC4-R and at least one other melanocortin receptor; and
selecting the compound as a candidate compound for said treatment if the compound has a Ki (nM) with respect to MC4-R that is at least about ten times lower than the Ki (nM) with respect to the at least one other melanocortin receptor. The at least one other melanocortin receptor may comprise MCL-R, MC3-R and MC5-R.

The method may further comprise the steps of:
determining specificity of the compound for MCL-R, MC3-R, MC4-R and MC5-R; and
selecting the compound as a candidate compound for said treatment if the compound is more specific for MC4-R that for MC1-R, MC3-R or MC5-R. Selecting the compound may comprise selecting the compound wherein the compound attenuates the binding of both α-MSH as to MC4-R and AgRP as to MC4-R by inhibiting, in a competitive inhibition assay, at least about 90% of the binding of each α-MSH and AgRP to MC4-R.

In one general method, selecting the compound may comprise selecting a compound having a Ki (nM) of less than about 10 as to both α-MSH binding to MC4-R and AgRP binding to MC4-R, or alternatively less than about 5 as to both α-MSH binding to MC4-R and AgRP binding to MC4-R. In the method, selecting the compound may comprise selecting a compound having a Ki (nM) as to α-MSH binding to MC4-R that is no more than about five times higher or lower than the Ki (nM) as to AgRP binding to MC4-R, or alternatively no more than about three times higher or lower than the Ki (nM) as to AgRP binding to MC4-R.

The method may further comprise the steps of:
determining the Ki (nM) of the compound at MC4-R;
determining the EC₅₀ of the compound with respect to MC4-R; and
selecting the compound If the compound has a Ki (nM) value that is half or less than half the EC₅₀ (nM) value. MC4-R agonist may be α-MSH or NDP-α-MSH. Selecting the compound may comprise selecting the compound If the compound has a Ki (nM) value that is at least five times less than the EC₅₀ (nM) value. Selecting the compound may comprise selecting the compound If the compound has a Ki (nM) value that is at least ten times less than the EC₅₀ (nM) value. Selecting the compound may comprise selecting the compound If the compound has a Ki (nM) value that is more than at least ten times less than the EC₅₀ (nM) value.

The method may further comprise the steps of:
determining the intrinsic activity of the compound based on maximal stimulation of adenylyl cyclase achievable by the compound in a MC4-R cell system where the maximal stimulation achieved by α-MSH or NDP-α-MSH is designated as an intrinsic activity of 1.0 (100%); and
selecting the compound If it has an intrinsic activity of less than about 0.1 (10%)

This method may further comprise the steps of:
determining the Ki (nM) of the compound at MC4-R;
determining the EC₅₀ of the compound with respect to MC4-R; and
selecting the compound as a candidate compound for said treatment if the compound has a Ki (nM) value that is half or less than half the EC₅₀ (nM) value. Here too the MC4-R agonist may be α-MSH or NDP-α-MSH. Selecting the compound may comprise selecting the compound If the compound has a Ki (nM) value that is at least five times less than the EC₅₀ (nM) value. Selecting the compound may comprise selecting the compound If the compound has a Ki (nM) value that is at least ten times less than the EC₅₀ (nM) value. Selecting the compound may comprise selecting the compound If the compound has a Ki (nM) value that is more than at least ten times less than the EC₅₀ (nM) value. Selecting the compound may comprise selecting the compound If the compound has a Ki (nM) value that is more than at least one hundred times less than the EC₅₀ (nM) value.

The general method further may comprise the steps of:

determining the functional status of the compound as an agonist, partial agonist, inactive or an antagonist in a MC4-R cell system; and

selecting the compound If it is an antagonist.

The general method may further comprise the steps of:

determining the functional status of the compound as an agonist, partial agonist, inactive or an antagonist in a MC4-R cell system; and

selecting the compound as a candidate compound for said treatment if it is inactive. Selecting the compound If it is inactive may comprise selecting the compound wherein intrinsic activity of the compound based on maximal stimulation of adenylyl cyclase achievable by the compound in a MC4-R cell system is less than about 0.1 (10%) where the maximal stimulation achieved by α-MSH or NDP-A-MSH is designated as an intrinsic activity of 1.0 (100%).

In yet another and distinct aspect, the invention provides a pharmaceutical composition for treating weight related disorders such as obesity and/or related energy homeostasis or feeding disorders characterized by excess weight gain, which compound does not induce or initiate a sexual response in a mammal, the composition comprising a substance that attenuates the binding of an agonist to a melanocortin receptor in a melanocortin receptor assay system and attenuates the binding of an inverse agonist to the same melanocortin receptor in the melanocortin receptor assay system, or a pharmaceutically acceptable salt thereof, and a pharmaceutically acceptable carrier, wherein the composition does not induce or initiate a sexual response in a mammal. The melanocortin receptor assay system may comprise an MC4-R assay system. The agonist may be alpha-melanocyte stimulating hormone (α-MSH) and the inverse agonist may be agouti-related protein (AgRP), such as wherein α-MSH is NDP-α-MSH and AgRP is AgRP (83-132). Preferably the substance has a Ki (nM) with respect to MC4-R that is at least about ten times lower than the Ki (nM) with respect to at least one other melanocortin receptor. The at least one other melanocortin receptor may comprise one or more of MC1-R, MC3-R and MC5-R.

In the pharmaceutical composition, the substance may be more specific for MC4-R than for MC1-R, MC3-R or MC5-R. The substance may attenuate the binding of both α-MSH as to MC4-R and AgRP as to MC4-R by inhibiting, in a competitive inhibition assay, at least about 90% of the binding of each α-MSH and AgRP to MC4-R. The substance has a Ki (nM) of less than about 10 as to both α-MSH binding to MC4-R and AgRP binding to MC4-R, or less than about 5 as to both α-MSH binding to MC4-R and AgRP binding to MC4-R. The substance may have a Ki (nM) as to α-MSH binding to MC4-R that is no more than about five times higher or lower than the Ki (nM) as to AgRP binding to MC4-R, is no more than about three times higher or lower than the Ki (nM) as to AgRP binding to MC4-R, or that is half or less than half of the EC₅₀ (nM) value of the substance with respect to MC4-R. The substance may have a Ki (nM) value that is at least five times less than the EC₅₀ (nM) value, at least ten times less than the EC₅₀ (nM) value, or more than at least ten times less than the EC₅₀ (nM) value. The substance may have an intrinsic activity of less than about 0.1 (10%) based on maximal stimulation of adenylyl cyclase achievable by the substance in a MC4-R cell system where the maximal stimulation achieved by α-MSH or NDP-α-MSH is designated as an intrinsic activity of 1.0 (100%).

The substance may be an antagonist in a MC4-R cell system or may be inactive in a MC4-R cell system. The substance is inactive if intrinsic activity of the substance based on maximal stimulation of adenylyl cyclase achievable by the compound in a MC4-R cell system is less than about 0.1 (10%) where the maximal stimulation achieved by α-MSH or NDP-α-MSH is designated as an intrinsic activity of 1.0 (100%).

In another and distinct aspect of the invention, there is provided a method of treating weight related disorders such as obesity and/or related energy homoeostasis or feeding disorders characterized by excess weight gain, comprising administration of a therapeutically effective amount of a compound that is an antagonist with respect to MC4-R. Preferably, the compound attenuates the binding of both an agonist and an inverse agonist to MC4-R.

In another and distinct aspect of the invention, there is provided a method of treating weight related disorders such as obesity and/or related energy homoeostasis or feeding disorders characterized by excess weight gain, comprising administration of a therapeutically effective amount of a compound that has a Ki(nM) at hMC4-R with respect to both NDP-α-MSH and AgRP of no more than about 50 (nM), and which has an intrinsic activity of less than about 0.3 (30%), based on maximal stimulation of adenylyl cyclase achievable by the compound in a MC4-R cell assay system where the maximal stimulation achieved by α-MSH or NDP-α-MSH is designated as an intrinsic activity of 1.0 (100%).

In another aspect, the invention provides a pharmaceutical composition for treating weight related disorders such as obesity and/or related energy homeostasis or feeding disorders characterized by excess weight gain, which composition does not substantially induce or initiate a sexual response in a mammal, the composition comprising a compound of the formula:

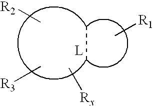

wherein:
L is a conformationally restricted ring system consisting of a single ring or bicyclic non-aromatic carbocyclic ring system, a single ring or bicyclic aromatic carbocyclic ring system, a single ring or bicyclic non-aromatic heterocyclic ring system or a single ring or bicyclic aromatic heterocyclic ring system, with the single ring comprising from 5 to about 9 atoms, and the bicyclic ring system comprising from 5 to about 9 atoms in each ring, where the dashed line represents the common bond between shared atoms of the two rings where L is a bicyclic ring system;
R₁ is a bond or a linker unit comprising from one to six backbone atoms selected from the group consisting of carbon (C), oxygen (O) and nitrogen (N) and an unsubstituted naphthalene group;
R₂ is a C₁ to C₆ aliphatic chain and a heteroatom unit with at least one cationic center, hydrogen (H) bond donor or hydrogen bond acceptor wherein at least one heteroatom is N, a C₁ to C₆ aliphatic amino acid side chain moiety or a neutral hydrogen bonding or positively charged amino acid side chain moiety;
R₃ comprises a bond or a linker unit and at least one carbocyclic aromatic ring; and
Rₓ comprises from zero to about three additional pendant groups;

or an enantiomer, stereoisomer or diastereoisomer thereof, or a pharmaceutically acceptable salt thereof; and

a pharmaceutically acceptable carrier;

wherein the compound attenuates the binding of both an agonist, including α-MSH, and an inverse agonist, including AgRP, to a MC4-R in a melanocortin receptor assay system.

In another aspect, the invention provides a pharmaceutical composition for treating weight related disorders such as obesity and related energy homeostasis or feeding disorders characterized by excess weight gain, which composition does not substantially induce or initiate a sexual response in a mammal, the composition comprising a compound of the formula:

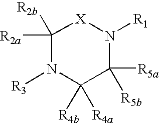

or an enantiomer, stereoisomer or diastereoisomer thereof, wherein
X is CH₂, C═O or C═S;
R₁ is -L₁-J;
One of R₂ₐ and R_{2b} is -L₂-W and the remaining of R₂ₐ and R_{2b} is hydrogen;
R₃ is -L₃-Q;
L₁ is a bond or a linker unit comprising from one to eight backbone atoms selected from the group consisting of carbon, sulfur, oxygen and nitrogen;
J is a ring structure selected from the group consisting of substituted or unsubstituted aromatic carbocyclic rings, substituted or unsubstituted non-aromatic carbocyclic rings, substituted or unsubstituted aromatic fused carbobicyclic ring groups, two substituted or unsubstituted aromatic carbocyclic rings wherein the rings are joined by a bond or —O—, and substituted or unsubstituted aromatic fused heterobicyclic ring groups; wherein in each instance the rings include 5 or 6 ring atoms;
L₂ is a bond or —(CH₂)_{y}—;
W is a heteroatom unit with at least one cationic center, hydrogen bond donor or hydrogen bond acceptor wherein at least one heteroatom is nitrogen or oxygen;
L₃ is a bond or a linker unit comprising from one to nine backbone atoms selected from the group consisting of carbon, sulfur, oxygen and nitrogen;
Q is a carbocyclic ring group comprising at least one aromatic ring;
one or two of R₄ₐ, R_{4b}, R₅ₐ, and R_{5b} are independently L₃-G and, if one of R₄ₐ, R_{4b}, R₅ₐ, and R_{5b} is L₃-G, then one of R₄ₐ, R_{4b}, R₅ₐ, and R_{5b} that is not L₃-G is optionally a C₁ to C₆ aliphatic linear or branched chain, and the remaining of R₄ₐ, R_{4b}, R₅ₐ, and R_{5b} are hydrogen, provided that at least one of R₄ₐ and R_{4b} and at least one of R₅ₐ and R_{5b} are hydrogen;
G is a ring structure selected from the group consisting of substituted or unsubstituted aromatic carbocyclic rings, substituted or unsubstituted non-aromatic carbocyclic rings, substituted or unsubstituted aromatic fused carbobicyclic ring groups, substituted or unsubstituted aromatic heterocyclic rings, substituted or unsubstituted non-aromatic heterocyclic rings, two substituted or unsubstituted aromatic carbocyclic rings wherein the rings are joined by a bond or —O—, and substituted or unsubstituted aromatic fused heterobicyclic ring groups, provided that if two of R₄ₐ, R_{4b}, R₅ₐ, and R_{5b} are L₃-G, then no more than one G comprises an aromatic ring; and
y is at each occurrence independently from 1 to 6;
or a pharmaceutically acceptable salt thereof; and

a pharmaceutically acceptable carrier;

wherein the compound attenuates the binding of both an agonist, such as α-MSH, and an inverse agonist, such as AgRP, to a MC4-R in a melanocortin receptor assay system.

In another aspect, the invention provides a pharmaceutical composition for treating weight related disorders such as obesity and related energy homeostasis or feeding disorders characterized by excess weight gain, which composition does not substantially induce or initiate a sexual response in a mammal, the composition comprising a compound of the formula:

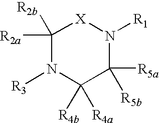

or an enantiomer, stereoisomer or diastereoisomer thereof, or a pharmaceutically acceptable salt thereof, wherein
X is CH₂, C═O or C═S;
R₁ is -L₁-J;
One of R₂ₐ and R_{2b} is -L₂-W and the remaining of R₂ₐ and R_{2b} is hydrogen;
R₃ is -L₃-Q;
L₁ is a bond or a linker unit comprising from one to eight backbone atoms selected from the group consisting of carbon, sulfur, oxygen and nitrogen;
J is a ring structure selected from the group consisting of substituted or unsubstituted aromatic carbocyclic rings, substituted or unsubstituted non-aromatic carbocyclic rings, substituted or unsubstituted aromatic fused carbobicyclic ring groups, two substituted or unsubstituted aromatic carbocyclic rings wherein the rings are joined by a bond or —O—, and substituted or unsubstituted aromatic fused heterobicyclic ring groups; wherein in each instance the rings include 5 or 6 ring atoms;
L₂ is a bond or —(CH₂)_{y}—;
W is a heteroatom unit with at least one cationic center, hydrogen bond donor or hydrogen bond acceptor wherein at least one heteroatom is nitrogen or oxygen;
L₃ is a bond or a linker unit comprising from one to nine backbone atoms selected from the group consisting of carbon, sulfur, oxygen or nitrogen;
Q is an aromatic carbocyclic ring selected from the group consisting of phenyl, substituted phenyl, naphthyl and substituted naphthyl;
one or two of R₄ₐ, R_{4b}, R₅ₐ, and R_{5b} are independently -L₂-W or a C₁ to C₆ aliphatic linear or branched chain and the remaining of R₄ₐ, R_{4b}, R₅ₐ, and R_{5b} are hydrogen, provided that at least one of R₄ₐ and R_{4b} and at least one of R₅ₐ and R_{5b} are hydrogen;
y is at each occurrence independently from 1 to 6; and

a pharmaceutically acceptable carrier;

wherein the compound attenuates the binding of both an agonist, such as α-MSH, and an inverse agonist, such as AgRP, to a MC4-R in a melanocortin receptor assay system.

In another aspect, the invention provides a pharmaceutical composition for treating weight related disorders such as obesity and related energy homeostasis or feeding disorders characterized by excess weight gain, which composition does not substantially induce or initiate a sexual response in a mammal, the composition comprising a peptide comprising the sequence S₁-S₂-S₃-S₄-S₅, wherein:
S₁ is heptanoyl, 2′-naphthylacetyl, 7′-amino-heptanoyl, 2′-chlorophenylacetyl, 3′-chlorophenylacetyl, 4′-chlorophenylacetyl, 4′-phenylbutylaminocarbonyl, 3′-phenylbutylaminocarbonyl, 4′-bromophenyl-acetyl, 3-4-dichlorophenyl-acetyl, 2,4-dichlorophenyl-acetyl, 4-biphenyl-acetyl, 2-naphthoyl, Ph-(CH₂)₂NH, 3′-phenylpropanecarbonyl, 2′-naphthoyl-Pip, 2′-naphthylacetyl, 2′-bromophenyl-acetyl, 4′-CF₃-phenyl-acetyl, 3′-CF₃-phenyl-acetyl, 2′-CF₃-phenyl-acetyl, 3′,5′-CF₃-phenylacetyl, 2′,5′-CF₃-phenylacetyl, 4′-Mephenyl-acetyl, 3′-Mephenyl-acetyl, 2′-Mephenyl-acetyl, 7′-aminoheptonoyl, beta-Ala, 4-aminoBytyl, 5-aminoValeryl, 6-aminoCaproyl, aminoTranexamyl, Cmpi or 3′4′—Cl₂-phenylacetyl;
S₂ is absent or is Ser(Bzl), Ala, D-Ala, beta-Ala, Val, Leu, Chg, Aib, Tle, 1-amino-1cyclohexanecarbonyl, Inp, CO(CH₂)₂NH, CO(CH₂)₂CO, Pip, MeThr(Bzl), Thr(Bzl) or D-Thr(Bzl);
S₃ is Phe, D-Phe, Phe(4-Cl), D-Phe(4-Cl), Phe(3-Cl), D-Phe(3-Cl), Phe(2-Cl), D-Phe(2-Cl), D-Phe(3,4-diCl), MePhe, D-MePhe, D-Tic, D-Tpi, D-Nal 2, Arg, D-Phe(3,4-F₂), D-Tiq, D-Me(homo)Phe or D-EtPhe;
S₄ is Arg, D-Arg, (Nlys)Gly, Trp, Lys, homoLys, Dpr(beta-Ala), alpha-(N-amidino-4′-piperidine)Gly, (4′-guanidino)Gly, (4′-guanidino)Phe, D-(4′-guanidino)Phe, beta-(N-amidino-4′-peperidine)Ala or homo-Ala-4′-pip(N-amidino); and
S₅ is Trp, Trp-OH, Trp-NH₂, Trp-Cys-NH₂, D-Trp, D-Trp-NH₂, Trp-Val-NH₂, 3′-Pya-NH₂, Phe-NH₂, MeTrp-NH₂, beta-Ala-Trp-NH₂, aminobutylamide, Nal 1-NH₂, D-Nal 1-NH₂, Nal 2-NH₂, D-Nal 2-NH₂, Tic-NH₂, D-Tic-NH₂, 1′-aminoindan, 1′-aminoindane-1-carboxyl-NH₂, Aic-NH₂, Atc-NH₂, Disc-NH₂, Tpi-NH₂, D-Tpi-NH₂, Tiq-NH₂, D-Tiq-NH₂, tryptamide, NMe-tryptamide, alpha-Me-tryptamide, 2′-(4″-methylphenyl)ethylamide, 3′,4′—Cl₂)phenylmethylamide, 3′-phenylpropylamide, 2′,4′-dichlorobenzylamide, 3′-(1H-imidazol)propylamide, 4-phenyl-piperidine-4-carbonamide, 3-phenyl-1-propylamide, 2,4-dichlorophenethylamide, S-(−)-1-(2-naphthyl)ethylamide, S-(−)-1-(1-naphthyl)ethylamide, 2′-methylbenzylamide, 4′-methylbenzylamide, 2′,2′-diphenylethylamide, 1-(2-pyridyl)piperazine, N-benzylmethylamide, histamide, R-(+)-1-(2-Naphthyl)ethylamide, Trp-Asp-NH₂, Trp-Asp-Phe-NH₂, Asp-Trp-NH₂, Ala-Trp-NH₂, Trp-Ala-NH₂, phenethylamide or Trp-Asp-OH;
or a pharmaceutically acceptable salt thereof; and

a pharmaceutically acceptable carrier;

wherein the compound attenuates the binding of both an agonist, including α-MSH, and an inverse agonist, including AgRP, to a MC4-R in a melanocortin receptor assay system.

In another aspect, the invention provides a pharmaceutical composition for treating weight related disorders such as obesity and related energy homeostasis or feeding disorders characterized by excess weight gain, which compound does not substantially induce or initiate a sexual response in a mammal, the composition comprising a compound having the structure:

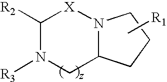

or a stereoisomer or pharmaceutically acceptable salt thereof,
wherein
R₁ is -L₁-J;
R₂ is (CH₂)_{y}—W;
R₃ is -L₂-Q;
L₁ is a linker selected from the group consisting of —(CH₂)_{y}—, —O—(CH₂)_{y}—, —O—, —NH—(CH₂)_{y}—, —(C═O)(CH₂)_{y}—, —(C═O)—O—(CH₂)_{y}—, and —CH₂(C═O)NH—;
J is a ring structure selected from the group consisting of substituted or unsubstituted aromatic carbocyclic rings, substituted or unsubstituted non-aromatic carbocyclic rings, substituted or unsubstituted aromatic fused carbobicyclic ring groups, substituted or unsubstituted aromatic carbocyclic ring groups wherein the rings are joined by a bond or —O—, and substituted or unsubstituted aromatic fused heterobicyclic ring groups; wherein in each instance the rings comprise 5 or 6 ring atoms;

W is a heteroatom unit with at least one cationic center, hydrogen bond donor or hydrogen bond acceptor wherein at least one atom is N;
L₂ is a linker selected from the group consisting of

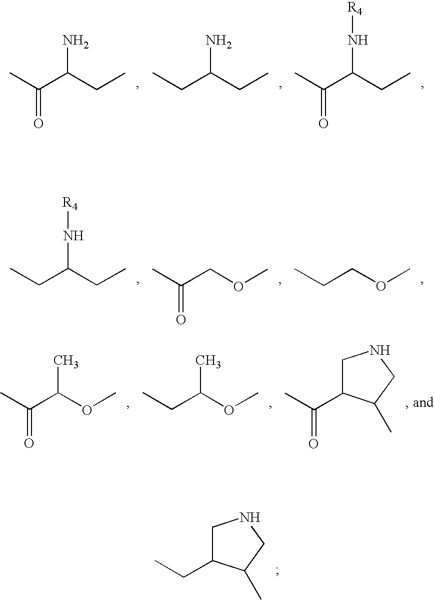

Q is an aromatic carbocyclic ring selected from the group consisting of phenyl, substituted phenyl, naphthyl and substituted naphthyl;
R₄ is a unit selected from the group consisting of an amine capping group, an amino acid residue, and an amino acid residue with an amine capping group;
X is CH₂ or C═O;
z is 0 or 1; and
y is at each occurrence independently from 1 to 6; and

a pharmaceutically acceptable carrier;

wherein the compound attenuates the binding of both an agonist, such as α-MSH, and an inverse agonist, such as AgRP, to a MC4-R in a melanocortin receptor assay system.

In another aspect, the invention provides a pharmaceutical composition for treating weight related disorders such as obesity and related energy homeostasis or feeding disorders characterized by excess weight gain, which compound does not substantially induce or initiate a sexual response in a mammal, the composition comprising a compound having the structure:

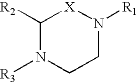

or a stereoisomer or pharmaceutically acceptable salt thereof, wherein
R₁ is -L₁-J or, if X is CH₂, is H or -L₁-J;
R₂ is (CH₂)_{y}—W or, if X is CH₂, is H or -L₁-J, on the proviso that not R₁ and R₂ are not both;
R₃ is -L₂-Q;
L₁ is a linker selected from the group consisting of —(CH₂)_{y}—, —O—(CH₂)_{y}—, —O—, —NH—(CH₂)_{y}—, —(C═O)(CH₂)_{y}—, —(C═O)—O—(CH₂)_{y}—, and —CH₂(C═O)NH—;
J is a ring structure selected from the group consisting of substituted or unsubstituted aromatic carbocyclic rings, substituted or unsubstituted non-aromatic carbocyclic rings, substituted or unsubstituted aromatic fused carbobicyclic ring groups, substituted or unsubstituted aromatic carbocyclic ring groups wherein the rings are joined by a bond or —O—, and substituted or unsubstituted aromatic fused heterobicyclic ring groups; wherein in each instance the rings comprise 5 or 6 ring atoms;
W is a heteroatom unit with at least one cationic center, hydrogen bond donor or hydrogen bond acceptor wherein at least one atom is N;
L₂ is a linker selected from the group consisting of

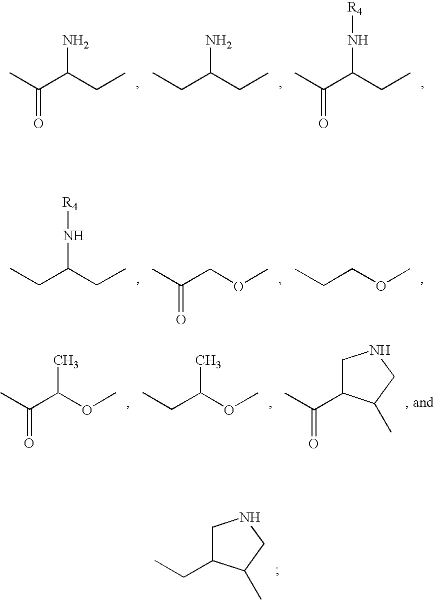

Q is an aromatic carbocyclic ring selected from the group consisting of phenyl, substituted phenyl, naphthyl and substituted naphthyl;
R₄ is H, —R₅ or —R₅-R₆;
R₅ is an amino acid residue or an amine capping group, provided that if R₆ is present,
R₅ is an amino acid residue;
R₆ is H or an amine capping group;
X is CH₂ or C═O; and
y is at each occurrence independently from 1 to 6; and

a pharmaceutically acceptable carrier;

wherein the compound attenuates the binding of both an agonist, such as α-MSH, and an inverse agonist, such as AgRP, to a MC4-R in a melanocortin receptor assay system.

One object of the present invention is to provide a melanocortin receptor-specific agent useful in treatment of obesity, the agent characterized in part in that it attenuates the binding of both α-MSH and AgRP to one or more melanocortin receptors.

Another object of the present invention is to provide a method for selection of a melanocortin receptor-specific agent useful in treatment of obesity, the method characterized in part in that an agent is selected which attenuates the binding of both α-MSH and AgRP to one or more melanocortin receptors.

Another object of the present invention is to provide a melanocortin receptor-specific agent useful in treatment of obesity, the agent characterized in part in that it attenuates the binding of both α-MSH and AgRP to one or more melanocortin receptors without substantial intrinsic activity determined by activation of adenylyl cyclase.

Another object of the present invention is to provide a method for selection of a melanocortin receptor-specific agent useful in treatment of obesity, the method characterized in part in that an agent is selected which attenuates the binding of both α-MSH and AgRP to one or more melanocortin receptors without substantial intrinsic activity determined by activation of adenylyl cyclase.

Another object of the present invention is to provide a melanocortin receptor-specific agent useful in treatment of obesity, the agent characterized in part in that it is efficacious for treatment of obesity without substantially inducing a sexual response, including without substantially inducing a penile erection response in a male.

Another object of the present invention is to provide a method for selection of a melanocortin receptor-specific agent useful in treatment of obesity, the method characterized in part in that an agent is selected which attenuates the binding of both α-MSH and AgRP to one or more melanocortin receptors without substantially inducing a sexual response, for example, without substantially inducing a penile erection response in a male.

Yet another object of the present invention is to provide a melanocortin receptor-specific agent useful in treatment of obesity that binds to MC4-R with high affinity but has low intrinsic activity in terms of cAMP accumulation.

Yet another object of the present invention is to provide a melanocortin receptor-specific agent that is an agonist or partial agonist and is useful in treatment of obesity that has a Ki (nM) at MC4-R, determined with respect to NDP-α-MSH, that is half or less than half, and preferably substantially less than half, of the EC₅₀ (nM) at MC4-R.

Yet another object of the present invention is to provide a method of treating obesity comprising administration of a melanocortin receptor-specific agent that attenuates the binding of both α-MSH and AgRP to melanocortin receptors.

Yet another object of the present invention is to provide a method of treating obesity comprising administration of a melanocortin receptor-specific agent that binds to MC4-R with high affinity but has low intrinsic activity in terms of cAMP accumulation, and is efficacious is treating obesity.

Yet another object of the present invention is to provide a method of treating obesity comprising administration of a melanocortin receptor-specific agent that has a Ki (nM) at MC4-R, determined with respect to NDP-α-MSH, that is half or less than half, and preferably substantially less than half, of the EC₅₀ (nM) for cAMP accumulation at MC4-R.

Yet another object of the present invention is to provide a method of treating obesity comprising administration of a melanocortin receptor-specific agent wherein there is no significant rebound effect or increase in body weight, following cessation of administration of the agent.

Yet another object of the present invention is to provide a method of treatment and an agonist or partial agonist melanocortin receptor-specific agent wherein the agent has a Ki (nM) at MC4-R, determined with respect to AgRP or AgRP (83-132), that is half or less than half, and preferably substantially less than half, of the EC₅₀ (nM) for cAMP accumulation at MC4-R.

Yet another object of the present invention is to provide a method of treatment and melanocortin receptor-specific agent useful in treatment of obesity that has high specific affinity for MC4-R, determined with respect to NDP-α-MSH, AgRP or AgRP (83-132) and is a neutral antagonist with respect to α-MSH, NDP-α-MSH or related analogs in functional cAMP accumulation assays.

Yet another object of the present invention is to provide a method of treatment and melanocortin receptor-specific agent useful in treatment of obesity that has high specific affinity for MC4-R, determined with respect to NDP-α-MSH, AgRP or AgRP (83-132), and is further characterized as a weak partial agonist, preferably with an EC₅₀ (nM) greater than at least 2-fold, and preferably substantially greater than 2-fold, of the Ki (nM) value for such agent, as determined in functional cAMP accumulation assays.

Yet another object of the present invention is to provide a method of treatment and melanocortin receptor-specific agent useful in treatment of obesity that has high specific affinity for MC4-R, determined with respect to NDP-α-MSH, AgRP or AgRP (83-132), yet is inactive or has an intrinsic activity of 0.35 or lower in a functional cAMP accumulation assay.

Yet another object of the present invention is to provide a method of treatment and melanocortin receptor-specific agent useful in treatment of obesity that has high specific affinity for MC4-R, determined with respect to NDP-α-MSH and AgRP or AgRP (83-132), and further that reverses the inverse agonism of the AgRP with respect to MC4-R stimulation.

According to one embodiment of the present invention, the methods provide compounds that bind to a putative common receptor binding site of α-MSH and AgRP, such as on MC4-R, preferably human MC4-R (hMC4-R), with high affinity yet without causing any activation, or alternatively causing low activation, of adenylyl cyclase activity. In one embodiment, the compounds are either inactive or are neutral antagonists with respect to α-MSH in cAMP accumulation assays. In another embodiment, the compounds are partial agonists, but functionally are similar to inactive compounds because the Ki (nM) values of the compounds are lower, preferably 2-fold or more lower, than corresponding EC₅₀ values, such that the compounds bind to receptors, such as MC4-R, and are efficacious for lowering food intake and body weight at low doses that are not efficacious in stimulating activation of adenylyl cyclase activity. The invention thus provides, in one embodiment, compounds that cause decrease in food intake and body weight by binding to a receptor, such as MC4-R, and thereby inhibiting the binding of endogenous AgRP to the receptor.

Yet another object of the present invention is to provide a melanocortin receptor-specific agent useful in treatment of obesity that binds to MC3-R with high affinity but has low intrinsic activity in terms of cAMP accumulation.

Yet another object of the present invention is to provide a method of treatment and an agonist or partial agonist melanocortin receptor-specific agent wherein the agent has a Ki (nM) at MC3-R, determined with respect to AgRP or AgRP (83-132), that is half or less than half, and preferably substantially less than half, of the EC₅₀ (nM) for cAMP accumulation at MC3-R.

Yet another object of the present invention is to provide a method of treatment and melanocortin receptor-specific agent useful in treatment of obesity that has high specific affinity for MC3-R, determined with respect to NDP-α-MSH, AgRP or AgRP (83-132) and is a neutral antagonist with respect to α-MSH, NDP-α-MSH or related analogs in functional cAMP accumulation assays.

Yet another object of the present invention is to provide a method of treatment and melanocortin receptor-specific agent useful in treatment of obesity that has high specific affinity for MC3-R, determined with respect to NDP-α-MSH, AgRP or AgRP (83-132), and is further characterized as a weak partial agonist, preferably with an EC₅₀ (nM) greater than at least 2-fold, and preferably substantially greater than 2-fold, of the Ki (nM) value for such agent, as determined in functional cAMP accumulation assays.

Yet another object of the present invention is to provide a method of treatment and melanocortin receptor-specific agent useful in treatment of obesity that has high specific affinity for MC3-R, determined with respect to NDP-α-MSH and AgRP or AgRP (83-132), yet is inactive or has an intrinsic activity of 0.35 or lower in a functional cAMP accumulation assay.

Yet another object of the present invention is to provide a method of treatment and melanocortin receptor-specific agent useful in treatment of obesity that has high specific affinity for MC3-R, determined with respect to NDP-α-MSH, AgRP or AgRP (83-132), and further that reverses the inverse agonism of the AgRP with respect to MC3-R stimulation.

The methods of this invention provide compounds that bind to a putative common receptor binding site of α-MSH and AgRP, such as on MC3-R, preferably human MC3-R, with high affinity yet without causing any activation, or alternatively causing low activation, of adenylyl cyclase activity. In one embodiment the compounds are either inactive or are neutral antagonists with respect to α-MSH in cAMP accumulation assays. In another embodiment the compounds are partial agonists, but function as inactive compounds in lowering food intake and body weight in vivo because the Ki (nM) values of the compounds are lower, preferably 2-fold or more lower, than corresponding EC₅₀ values, such that the compounds bind to receptors, such as MC3-R, and are efficacious for lowering food intake and body weight at low doses that are not efficacious in stimulating activation of adenylyl cyclase activity. The invention thus provides, in one embodiment, compounds that cause decrease in food intake and body weight by binding to a receptor, such as MC3-R, and thereby inhibiting the binding of endogenous AgRP to the receptor.

Other objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 is a graph showing of cAMP accumulation in HEK-293/MCR-4 cells of the compound of Example 11.

FIG. 2 is a graph showing the antagonistic activity of the compound of Example 12 in HEK-293 cells expressing MCR-4 receptors. The pA₂ value derived from the Schild plot is 7.52.

FIG. 3 is a graph of inverse agonism of the compound of Example 13 in HEK-293 cells expressing MC4-R receptors.

FIG. 4 is a graph of the antagonistic activity of the compound of Example 4 on AgRP induced inverse agonism in HEK-2931MC4-R cells.

FIG. 5 is a graph showing the inhibition by AgRP of forskolin-stimulated cAMP levels in HEK-293 cells expressing MC4-R receptors.

FIGS. 6A and 6B are graphs showing the reversal of AgRP inhibition in forskolin-stimulated cAMP levels in HEK-293 cells expressing MC4-R receptors by use of the compound of Example 28, with FIG. 6A showing results over the range of 0 to 600 pmole/10⁶ cells of cAMP accumulation, and

FIG. 6B showing results over the range of 0 to 2000 pmole/10⁶ cells of cAMP accumulation.

FIGS. 7A and 7B are graphs showing the reversal of AgRP inhibition in forskolin-stimulated cAMP levels in HEK-293 cells over-expressing MC4-R receptors by use of the compound of Example 26, with FIG. 7A showing results over the range of 0 to 100 pmole/10⁶ cells of cAMP accumulation, and FIG. 7B showing results over the range of 0 to 2000 pmole/10⁶ cells of cAMP accumulation.

FIG. 8 is a graph showing the reversal of AgRP-induced decrease in the levels of cAMP in HEK-293 cells that express mutant L250Q MC4 receptors by use of the compound of Example 4.

### DETAILED DESCRIPTION OF THE INVENTION

In one broad aspect, the invention provides methods for treatment of obesity and related energy homeostasis diseases, conditions and syndromes, together with methods for the identification and selection of compounds with utility for treatment of obesity and related energy homeostasis diseases, conditions and syndromes. In a related aspect, the invention provides the use of a variety of specified melanocortin receptor-specific compounds that may be employed in the treatment of obesity and related energy homeostasis diseases, conditions and syndromes. In yet another related aspect, the invention provides a variety of combination and multiple agent therapies that may be employed for treatment of obesity and related energy homeostasis diseases, conditions and syndromes, such combination or multiple agent therapies including at least one compound identified or described by the methods set forth herein.

In the practice of the method of this invention, a melanocortin receptor-specific agent, and preferably an agent specific for MC4-R, is selected, which agent is characterized in that: (a) the agent attenuates the binding of both alpha-melanocyte stimulating hormone (α-MSH) and agouti-related protein (AgRP) to melanocortin receptors; (b) optionally, the agent is further characterized in having a low efficacy, such that the agent is a weak agonist, weak antagonist, neutral antagonist, inverse agonist or protean agonist at MC4-R, and preferably has an efficacy of less than about 50%, more preferably less than about 30%, and most preferably less than about 10%; and, (c) optionally, the agent is further characterized in that the agent has a Ki (nM) at MC4-R, determined with respect to NDP-α-MSH, that is half or less than half, and preferably substantially less than half, of the EC₅₀ (nM) at MC4-R.

The melanocortin receptor-specific agents disclosed herein, and the methods for selecting such agents, are further characterized in that the agents in general do not significantly induce a sexual response in a mammal, such as a penile erection response in a male. A number of melanocortin-receptor specific compounds and agents heretofore evaluated for use in treatment of obesity and related conditions have had unacceptable side effects relating to initiation or induction of a sexual response, including a penile erection response in males. In part, compounds selected by methods of the invention do not generally induce a sexual response.

The methods disclosed herein may be employed with any compound believed or suspected to bind to a melanocortin receptor, preferably to bind to MC4-R, and more preferably to bind to hMC4-R. Thus the methods and this invention may be employed with peptide, peptidomimetic, metallopeptide, small molecule, ring-core small molecule and other compounds known in the art, including compounds hereafter developed. While the invention has been exemplified with the compounds disclosed herein, the invention is not limited to such compounds, and may be applied to other compounds, which compounds may significantly differ from the compounds disclosed herein.

With respect to the compounds disclosed herein, the methods of synthesis and purification, methods of testing, formulas and definitions of classes of compounds and specific compounds disclosed in the following patent applications are relevant to the practice of the invention: U.S. patent application Ser. No. 11/557,408, entitled N-Alkylated Cyclic Peptide Melanocortin Agonists, filed on Nov. 7, 2006; U.S. patent application Ser. No. 11/464,069, entitled Melanocortin Receptor-Specific piperazine and Keto-piperazine Compounds, filed on Aug. 11, 2006; U.S. patent application Ser. No. 11/464,051, entitled Substituted Melanocortin Receptor-Specific Single Acyl piperazine Compounds, filed on Aug. 11, 2006; U.S. patent application Ser. No. 11/464,053, entitled Melanocortin Receptor-Specific piperazine Compounds with Diamine Groups, filed on Aug. 11, 2006; U.S. patent application Ser. No. 11/110,060, entitled Substituted Melanocortin Receptor-Specific piperazine Compounds, filed on Apr. 19, 2005; U.S. patent application Ser. No. 11/099,814, entitled Substituted Melanocortin Receptor-Specific piperazine Compounds, filed on Apr. 5, 2005; U.S. patent application Ser. No. 11/040,838, entitled Thieno[2,3-d]Pyrimidine-Z4-Dione Melanocortin-Specific Compounds, filed on Jan. 21, 2005; U.S. patent application Ser. No. 11/036,282, entitled Naphthlalene-Containing Melanocortin Receptor-Specific Small Molecules, filed on Jan. 14, 2005; U.S. patent application Ser. No. 10/837,519, entitled Melanocortin Receptor-Specific Compounds, filed on Apr. 30, 2004; U.S. patent application Ser. No. 10/762,079, entitled piperazine Melanocortin-Specific Compounds, filed on Jan. 21, 2004, and issued as U.S. Patent No. 7,354,923 on Apr. 8, 2008; U.S. patent application Ser. No. 10/761,889, entitled Bicyclic Melanocortin-Specific Compounds, filed on Jan. 21, 2004, and issued as U.S. Pat. No. 7,326,707 on Feb. 5, 2008; International Patent Application PCT/US02/22196, entitled Linear and Cyclic Melanocortin Receptor-Specific Peptides, filed on Nov. 7, 2002; International Patent Application PCT/US02/25574, entitled Peptidomimetics of Biologically Active Molecules, filed on Aug. 12, 2002; and International Patent Application PCT/US01/50075, entitled Identification of Target-Specific Folding Sites in Peptides and Proteins, filed on Dec. 19, 2001. The specification and claims of each of the foregoing patent applications is incorporated herein by reference.

Definitions. Before proceeding further with the description of the invention, certain terms are defined as set forth herein.

The “amino acid” and “amino acids” used in this invention, and the terms as used in the specification and claims, include the known naturally occurring protein amino acids, which are referred to by both their common three letter abbreviation and single letter abbreviation. See generally Synthetic Peptides: A User's Guide, G A Grant, editor, W.H. Freeman & Co., New York, 1992, the teachings of which are incorporated herein by reference, including the text and table set forth at pages 11 through 24. As set forth above, the term “amino acid” also includes stereoisomers and modifications of naturally occurring protein amino acids, non-protein amino acids, post-translationally modified amino acids, enzymatically synthesized amino acids, derivatized amino acids, constructs or structures designed to mimic amino acids, and the like. Modified and unusual amino acids are described generally in Synthetic Peptides: A User's Guide, cited above; Hruby V J, Al-obeidi F and Kazmierski W: Biochem J 268:249-262, 1990; and Toniolo C: Int J Peptide Protein Res 35:287-300, 1990; the teachings of all of which are incorporated herein by reference.

The term “amino acid side chain moiety” used in this invention includes any side chain of any amino acid, as the term “amino acid” is defined herein, including any derivative of an amino acid side chain moiety, as the term “derivative” is defined herein. Therefore, this includes the side chain moiety present in naturally occurring amino acids. It further includes side chain moieties in modified naturally occurring amino acids, such as glycosylated amino acids. It further includes side chain moieties in stereoisomers and modifications of naturally occurring protein amino acids, non-protein amino acids, post-translationally modified amino acids, enzymatically synthesized amino acids, derivatized amino acids, constructs or structures designed to mimic amino acids, and the like. For example, the side chain moiety of any amino acid disclosed herein is included within the definition of an amino acid side chain moiety.

The “derivative” of an amino acid side chain moiety includes any modification to or variation in any amino acid side chain moieties, including a modification of naturally occurring amino acid side chain moieties. By way of example, derivatives of amino acid side chain moieties include straight chain or branched, cyclic or noncyclic, substituted or unsubstituted, and saturated or unsaturated alkyl, aryl or aralkyl moieties.

The following abbreviations for amino acids, amino acid side chain moieties and derivatives and constituents thereof have the meanings giving, it being understood that any amino acid may be in either the L- or D-configuration:

Abu—gamma-amino butyric acid

2-Abz—2-amino benzoic acid

3-Abz—3-amino benzoic acid

4-Abz—4-amino benzoic acid

Achc—1-amino-cyclohexane-1-carboxylic acid

Acpc—1-amino-cyclopropane-1-carboxylic acid

12-Ado—12-amino dodecanoic acid

Aib—alpha-aminoisobutyric acid

1-Aic—2-aminoindane-1-carboxylic acid

2-Aic—2-aminoindane-2-carboxylic acid

6-Ahx—6-amino hexanoic acid

Beta-Ala—Bate-alanine

Amb—4-(aminomethyl)-benzoic acid

Amc—4-(aminomethyl)-cyclohexane carboxylic acid

7′-amino-heptanoyl—NH₂—(CH₂)₆CO—

8-Aoc—8-amino octanoic acid

Arg(Tos)—N^{G}-para-tosyl-arginine

Asp(anilino)—beta-anilino-aspartic acid

Asp(3-Cl-anilino)—beta-(3-chloro-anilino)-aspartic acid

Asp(3,5-diCl-anilino)—beta-(3,5-dichloro anilino)-aspartic acid

Atc—2-aminotetralin-2-carboxylic acid

11-Aun—11-amino undecanoic acid

AVA—5-amino valeric acid

Beta-hHyp(Bzl)—Beta-(O-benzyl)-homohydroxyproline

Beta-hSer(Bzl)—Beta-(O-benzyl)-homoserine

Bip—biphenylalanine

Bzl—benzyl

Bz—benzoyl

Cha—cyclohexylalanine

Chg—cyclohexylglycine

Cmpi—4-caboxymethyl-piperazine

Cys(Bzl)—S-benzyl-cysteine

Dip—3,3-diphenylalanine

Disc—1,3-dihydro-2H-isoindolecarboxylic acid

Dpr(beta-Ala)—N^{beta}-(3-aminopropionyl)-alpha, beta-diaminopropionic acid

Et—ethyl

GAA—epsilon-guanidino acetic acid

GBzA—4-guanidino benzoic acid

B-Gpa—3-guanidino propionic acid

GVA(Cl)—beta-chloro-epsilon-guanidino valeric acid

Heptanoyl—CH₃—(CH₂)₅CO

hPhe—homophenylalanine

hSer—homoserine

Hyp—hydroxy proline

hHyp—homo hydroxy proline

Hyp(Bzl)—O-benzyl-hydroxyproline

Hyp(2-naphthly)—O-2′ naphthyl-hydroxyproline

Hyp(Phenyl)—O-phenyl-hydroxyproline

Idc—indoline-2-carboxylic acid

Igl—indanylglycine

Inp—isonipecotic acid

Lys(Z)—N-epsilon-benzyloxycarbonyl-lysine

Me—methyl

Nal 1—3-(1-naphthyl)alanine

Nal 2—3-(2-naphthyl)alanine

(N-Bzl)Nal 2-N-benzyl-3-(2-naphthyl) alanine

2-Naphthylacetyl—2-naphthyl-CH₂CO—

(Nlys)Gly—N-(4-aminobutyl)-glycine

(N-PhEt)Nal 2-N(2-phenylethyl)-3-(2-naphthyl) alanine

OcHx—cyclohexyl ester

Phg—phenylglycine

Phe(4-F)— para-fluoro-phenylalanine

Phe(4-Br)—4-bromo-phenylalanine

Phe(4-CF₃)—4-trifluoromethyl-phenylalanine

Phe(4-Cl)—4-chloro-phenylalanine

Phe(3-Cl)—3-chloro-phenylalanine

Phe(2-Cl)—2-chloro-phenylalanine

Phe(2,4-diCi)—2,4,-dichloro-phenylalanine

Phe(2,4-diF)—2,4-difluoro-phenylalanine

Phe(3,4-diCl)—3,4,-dichloro-phenylalanine

Phe(5-Cl)—5-chloro-phenylalanine

Phe(2-Cl,4-Me)—2-chloro-4-methyl-phenylalanine

Phe(2-Me,4-Cl)—4-chloro-2-methyl-phenylalanine

Phe(2-F, 4-Cl)—4-chloro-2-fluoro-phenylalanine

Phe(2,4-diMe)—2,4-dimethyl-phenylalanine

Phe(2-Cl,4-CF₃)—2-chloro-4-trifluoromethyl-phenylalanine

Phe(3,4-diF)—3,4,-difluoro-phenylalanine

Phe(4-I)—4-iodo-phenylalanine

Phe(3,4-di-OMe)—3,4,-dimethoxy-phenylalanine

Phe(4-Me)—4-methyl-phenylalanine

Phe(4-OMe)—4-methoxy-phenylalanine

Phe(4-NC)—4-cyano-phenylalanine

Phe(4-NO₂)—4-nitro-phenylalanine

Pip—pipecolic acid

Pr—propyl

Pr-i—isopropyl

4-phenylPro—4-phenyl-pyrrolidin-2-carboxylic acid

5-phenyl Pro—5-phenyl-pyrrolidin-2-carboxylic acid

3-Pya—3-pyridylalanine

Pyr—pyroglutamic acid

Qal(2′)—beta-(2-quinolyl)-alanine

Sal—3-styrylalanine

Sar—sarcosine

Ser(Bzl)—O-benzyl-serine

Ser(2-Naphthyl)—O-2-Naphthyl-serine

Ser(Phenyl)—O-2-Phenyl-serine

Ser(4-Cl-Phenyl)—O-4-Cl-Phenyl-serine

Ser(2-Cl-Phenyl)—O-2-Cl-Phenyl-serine

Ser(p-Cl-Bzl)—O-4-Cl-Benzyl-serine

Thr(Bzl)—O-Benzyl-threonine

Thr(2-Naphthyl)—O-(2-naphthyl)-threonine

Thr(Phenyl)—O-phenyl-threonine

Thr(4-Cl-Phenyl)—O-(4-Cl-phenyl)-threonine

Thr(2-Cl-Phenyl)—O-(2-Cl-phenyl)-threonine

Beta-homoThr(Bzl)—O-Benzyl-bate-homothreonine

Tic—1,2,3,4-tetrahydroisoquinoline-3-carboxylic acid

Tiq—1,2,3,4-tetrahydroisoquinoline-1-carboxylic acid

Tle—tert-butylalanine

Tpi—1,2,3,4-tetrahydronorharman-3-carboxylic acid

Tyr(Bzl)—O-benzyl-tyrosine

Tyr(2,6-DiCl-Bzl)—O-(2,6 dichloro)benzyl-tyrosine

Conventional amino acid residues have their conventional meaning as given in Chapter 2400 of the Manual of Patent Examining Procedure, 8^{th} Ed. Thus, “NIe” is norleucine, “Asp” is aspartic acid, “His” is histidine, “D-Phe” is D-phenylalanine, “Arg” is arginine, “Trp” is tryptophan, “Lys” is lysine, “Gly” is glycine, “Pro” is praline, “Tyr” is tyrosine, “Ser” is serine and so on.

The term “homolog” includes, without limitation, (a) a D-amino acid residue or side chain substituted for an L-amino acid residue side chain, (b) a post-translationally modified residue or side chain substituted for the residue or side chain, (c) a non-protein or other modified amino acid residue or side chain based on another such residue or side chain, such as phenylglycine, homophenylalanine, ring-substituted halogenated, and alkylated or arylated phenylalanines for a phenylalanine residue, diamino proionic acid, diamino butyric acid, ornithine, lysine and homoarginine for an arginine residue, and the like, and (d) any amino acid residue or side chain, coded or otherwise, or a construct or structure that mimics an amino acid residue or side chain, and which has at least a similarly charged side chain (neutral, positive or negative), preferably a similar hydrophobicity or hydrophilicity, and preferably a similar side chain in terms of being a saturated aliphatic side chain, a functionalized aliphatic side chain, an aromatic side chain or a heteroaromatic side chain.

The term “alkene” includes unsaturated hydrocarbons that contain one or more double carbon-carbon bonds. Examples of such alkene groups include ethylene, propene, and the like.

The term “alkenyl” includes a linear monovalent hydrocarbon radical of two to six carbon atoms or a branched monovalent hydrocarbon radical of three to six carbon atoms containing at least one double bond; examples thereof include ethenyl, 2-propenyl, and the like.

The “alkyl” groups specified herein include those alkyl radicals of the designated length in either a straight or branched configuration. Examples of such alkyl radicals include methyl, ethyl, propyl, isopropyl, butyl, sec-butyl, tertiary butyl, pentyl, isopentyl, hexyl, isohexyl, and the like.

The term “alkynal” includes a linear monovalent hydrocarbon radical of two to six carbon atoms or a branched monovalent hydrocarbon radical of three to six carbon atoms containing at least one triple bond; examples thereof include ethynyl, propynal, butynyl, and the like.

The term “aryl” includes a monocyclic or bicyclic aromatic hydrocarbon radical of 6 to 12 ring atoms, and optionally substituted independently with one or more substituents selected from alkyl, haloalkyl, cycloalkyl, alkoxy, alkylhio, halo, nitro, acyl, cyano, amino, monosubstituted amino, disubstituted amino, hydroxy, carboxy, or alkoxy-carbonyl. Examples of an aryl group include phenyl, biphenyl, naphthyl, 1-naphthyl, and 2-naphthyl, derivatives thereof, and the like.

The term “aralkyl” includes a radical-R^{a}R^{b} where R^{a} is an alkylene (a bivalent alkyl) group and R^{b} is an aryl group as defined above. Examples of aralkyl groups include benzyl, phenylethyl, 3-(3-chlorophenyl)-2-methylpentyl, and the like.

The term “aliphatic” includes compounds with hydrocarbon chains, such as for example alkanes, alkenes, alkynes, and derivatives thereof.

The term “acyl” includes a group RCO—, where R is an organic group. An example is the acetyl group CH₃CO—.

A group or aliphatic moiety is “acylated” when an alkyl or substituted alkyl group as defined above is bonded through one or more carbonyl [—(C═O)—] groups.

An “omega amino derivative” includes an aliphatic moiety with a terminal amino group. Examples of omega amino derivatives include aminoheptanoyl and the amino acid side chain moieties of ornithine and lysine.

The term “heteroaryl” includes mono- and bicyclic aromatic rings containing from 1 to 4 heteroatoms selected from nitrogen, oxygen and sulfur. 5- or 6-membered heteroaryl are monocyclic heteroaromatic rings; examples thereof include thiazole, oxazole, thiophene, furan, pyrrole, imidazole, isoxazole, pyrazole, triazole, thiadiazole, tetrazole, oxadiazole, pyridine, pyridazine, pyrimidine, pyrazine, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine and thienopyridine.

An “amide” includes compounds that have a trivalent nitrogen attached to a carbonyl group (—CO.NH₂), such as methylamide, ethylamide, propylamide, and the like.

An “imide” includes compounds containing an imido group (—CO.NH.CO—).

An “amine” includes compounds that contain an amino group (—NH₂).

A “nitrile” includes compounds that are carboxylic acid derivatives and contain a (—CN) group bound to an organic group.

An amino acid side chain moiety is “hydrogen bonding” when the side chain includes hydrogen donors or alternatively hydrogen acceptors.

An “amine capping group” includes any terminal group attached through a terminal amine, including but not limited to any omega amino derivative, acyl group or terminal aryl or aralkyl including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 12-Ado, 7′-amino heptanoyl, 6-Ahx, Amc or 8-Aoc, as well as terminal groups such as polyethylene glycol (PEG) with an average or formula molecular weight of between 100 and 10,000, optionally a PEG carboxylic acid derivative capable of forming a covalent bond with a terminal amine.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing or otherwise combining a compound of the present invention and one or more pharmaceutically acceptable carriers, and optionally one or more pharmaceutically active ingredients and agents.

A variety of chemicals and compounds are employed in this invention, and the following abbreviations have the meanings given:
AcOH acetic acid
Boc tertiary butyloxycarbonyl
Cbz benzyloxycarbonyl
DCM dichloromethane
DEAD diethyl azodicarboxylate
DIAD diisopropyl azodicarboxylate
DIC 1,3-diisopropylcarbodiimide
DMF N,N-dimethylformamide
DMSO dimethyl sulfoxide
EtOAc ethyl acetate
Fmoc 9-fluorenylmethoxycarbonyl
HBTU 2-(1H-benzotriazole-1-yl)-1,1,3,3-tetramethyluronium hexafluorophosphate
HEPES 4-(2-hydroxyethyl)1-piperazineethanesulfonic acid
HOAt 1-hydroxy-7-azabenzotriazole
IBCF isobutyl chloroformate
LAH lithium aluminum hydride
NMM N-methyl-morpholine
NMP 1-methyl-2-pyrrolidinone
Prt A protecting group, such as Boc, Cbz or Fmoc
TBTU 2-(1H-Benzotriazole-1-yl)-1,1,3,3-tetramethyluronium tetrafluoroborate
TEA triethylamine
TFA trifluoroacetic acid
THF tetrahydrofuran
TPP triphenylphosphine

A “tri-substituted piperazine”, as used herein, is a piperazine compound or derivative thereof wherein a group other than solely H, and preferably including an amino acid residue or an amino acid side chain moiety, is attached to each ring N member, and further wherein a group other than solely H, O, S or a halogen, preferably including an amino acid side chain moiety, is attached to one ring C member.

A “tetra-substituted piperazine” or “penta-substituted piperazine”, as used herein, is a piperazine compound or derivative thereof wherein a group other than solely H, and preferably including an amino acid residue or an amino acid side chain moiety, is attached to each ring N member, and further wherein a group other than solely H, O, S or a halogen, preferably including an amino acid side chain moiety, is attached to two ring C members for a tetra-substituted piperazine and to three ring C members for a penta-substituted piperazine.

By a melanocortin receptor “agonist” is meant an endogenous or drug substance or compound, including compounds disclosed herein, that can interact with a melanocortin receptor and initiate a pharmacological response, including but not limited to adenylyl cyclase activation, characteristic of the melanocortin receptor. By a melanocortin receptor “antagonist” is meant a drug or a compound, including compounds disclosed herein, that opposes the melanocortin receptor-associated responses normally induced by a melanocortin receptor agonist agent, but without itself initiating a pharmacological response characteristic of the melanocortin receptor, such as increasing or decreasing adenylyl cyclase activation. By a melanocortin receptor “inverse agonist” is meant a drug or a compound, including compounds disclosed herein, which is an antagonist with respect to an agonist, and which by itself induces or initiates a pharmacological response characteristic of the melanocortin receptor, such as reducing basal or constitutive adenylyl cyclase activation. By a melanocortin receptor “protean agonist” is meant a drug or a compound, including compounds disclosed herein, which acts as either an inverse agonist or an agonist, depending on the constitutive activity of the MC4-R, either promoting a switch to a less active conformation or enriching the active conformation. In general, inverse and protean agonists are discussed at length in Kenakin, T. Inverse, protean, and ligand-selective agonism: matters of receptor conformation. The FASEB Journal 15:598-611, 2001, incorporated here by reference as if set forth in full.

By “α-MSH” is meant the peptide Ac-Ser-Tyr-Ser-Met-Glu-His-Phe-Arg-Trp-Gly-Lys-Pro-Val-NH₂ (SEQ ID NO:1) and analogs and homologs thereof, including without limitation NDP-α-MSH.

By “NDP-α-MSH” is meant the peptide Ac-Ser-Tyr-Ser-Nle-Glu-His-D-Phe-Arg-Trp-Gly-Lys-Pro-Val-NH₂ and analogs and homologs thereof.

By “AgRP” is meant Agouti-related protein or a biologically active fragment of Agouti protein, and for applications relating to human AgRP, including the truncated variants AgRP (83-132) and AgRP (87-132). AgRP includes proteins or fragments thereof made by purification of biological materials, synthetic methodologies or recombinant methodologies, and includes the sequences and constructs, including analogs, homologs and variants thereof, disclosed generally in Yang, Y.-K., Thompson, D. A., Dickinson, C. J., Wilken, J., Barsh, G. S., Kent, S. B. H., Gantz, I. Characterization of agouti-related protein binding to melanocortin receptors. Molecular Endocrinology 13:148-155, 1999; and Ollmann, M. M., Wilson, B. D., Yang, Y.-K., Kerns, J. A., Chen, Y. Gantz, I., Barsh, G. S. Science 278:135-138, 1997. AgRP further includes other agouti-related peptides and proteins which are or may be inverse agonists, including without limitation agouti signaling peptide.

By “AgRP (83-132)” is meant a biologically active fragment of AgRP containing the amino acids at positions 83 to 132 of human AgRP (Ser-Ser-Arg-Arg-Cys-Val-Arg-Leu-His-Glu-Ser-Cys-Leu-Gly-Gln-Gln-Val-Pro-Cys-Cys-Asp-Pro-Cys-Ala-Thr-Cys-Tyr-Cys-Arg-Phe-Phe-Asn-Ala-Phe-Cys-Tyr-Cys-Arg-Lys-Leu-Gly-Thr-Ala-Met-Asn-Pro-Cys-Ser-Arg-Thr (SEQ ID NO:2). AgRP (83-132) is included within the definition of AgRP given here.

By “AgRP (87-132)” is meant a biologically active fragment of AgRP containing the amino acids at positions 87 to 132 of human AgRP (Cys-Val-Arg-Leu-His-Glu-Ser-Cys-Leu-Gly-Gln-Gln-Val-Pro-Cys-Cys-Asp-Pro-Cys-Ala-Thr-Cys-Tyr-Cys-Arg-Phe-Phe-Asn-Ala-Phe-Cys-Tyr-Cys-Arg-Lys-Leu-Gly-Thr-Ala-Met-Asn-Pro-Cys-Ser-Arg-Thr (SEQ ID NO:3). AgRP (87-132) is included within the definition of AgRP given here.

By “attenuates” is meant that a compound or drug prevents or inhibits the binding of either AgRP or α-MSH to melanocortin receptors, and preferably MC4-R, or decreases the binding affinity of either AgRP or α-MSH to melanocortin receptors, and preferably MC4-R. Compounds of this invention attenuate the binding of both AgRP and α-MSH to melanocortin receptors, preferably MC4-R. For attenuation of AgRP binding, it is preferable if the compound being tested inhibits at least about 70% of AgRP binding, such as iodinated AgRP (83-132) binding in a competitive inhibition assay, and more preferably inhibits at least about 85% of AgRP binding, and most preferably inhibits at least about 90% or greater of AgRP binding. For attenuation of α-MSH binding, it is preferable if the compound being tested inhibits at least about 70% of α-MSH binding, such as iodinated NDP-α-MSH binding in a competitive inhibition assay, and more preferably inhibits at least about 85% of α-MSH, and most preferably inhibits at least about 95% or greater of α-MSH. The percent inhibition of binding can be readily determined by those skilled in the art by competition and other inhibition assays, including the methods disclosed herein and other comparable methods.

By “EC₅₀” is meant the molar concentration of an agonist, including a partial agonist, which produced 50% of the maximum possible response for that agonist. By way of example, a test compound which, at a concentration of 72 nM, produces 50% of the maximum possible response for that compound as determined in a cAMP assay in an MC4-R cell expression system, has an EC₅₀ of 72 nM. Unless otherwise specified, the molar concentration associated with an EC₅₀ determination is in nanomoles (nM).

By “Ki (nM)” is meant the equilibrium inhibitor dissociation constant representing the molar concentration of a competing compound that binds to half the binding sites of a receptor at equilibrium in the absence of radioligand or other competitors. In general, the Ki is inversely correlated to the affinity of the compound for the receptor, such that if the Ki is low, the affinity is high. Ki may be determined using the equation of Cheng and Prusoff (Cheng Y., Prusoff W. H., Biochem. Pharmacol. 22: 3099-3108,1973):

$$Ki = \frac{{EC}_{50}}{1 + \frac{[ ligand ]}{{K}_{D}}}$$

where “ligand” is the concentration of radioligand and K_{D} is an inverse measure of receptor affinity for the radioligand which produces 50% receptor occupancy by the radioligand. Unless otherwise specified, the molar concentration associated with a Ki determination is in nM. Ki may be expressed in terms of specific receptors (e.g., MC1-R, MC3-R, MC4-R or MC5-R) and specific ligands (e.g., either iodinated AgRP or α-MSH).

By “inhibition” is meant the percent attenuation, or decrease in receptor binding, in a competitive inhibition assay compared to a known standard. Thus, by “inhibition at 1 μM (NDP-α-MSH)” is meant the percent decrease in binding of NDP-α-MSH by addition of a determined amount of the compound to be tested, such as 1 μM of a test compound, such as under the assay conditions hereafter described. By way of example, a test compound that does not inhibit binding of NDP-α-MSH has a 0% inhibition, and a test compound that completely inhibits binding of NDP-α-MSH has a 100% inhibition. By “inhibition at 1 μM AgRP (83-132)” is meant the percent decrease in binding of AgRP (83-132) by addition of a determined amount of the compound to be tested, such as 1 μM of a test compound, such as under the assay conditions hereafter described. Typically, as described hereafter, a radio assay is used for competitive inhibition testing, such as with 1125-labeled NDP-α-MSH or AgRP (83-132). However, other methods of testing competitive inhibition are known, including use of label or tag systems other than radioisotopes, and in general any method known such as those known in the art for testing competitive inhibition may be employed in this invention. It may thus be seen that “inhibition” is one measure to determine whether a test compound attenuates binding of either AgRP or α-MSH to melanocortin receptors.

By “pA₂” is meant a logarithmic measure of the potency of a compound that is an antagonist, including a partial antagonist, or the negative log of the concentration of the antagonist which produces a 2-fold shift in the concentration-response curve for an agonist. The pA₂ value is typically determined by the intercept on the y-axis of the extrapolated line in a Schild plot plotting the log (concentration ratio −1) against the log (antagonist) concentration. See generally Schild, H. O. pA, a new scale for the measurement of drug antagonism. Br. J. Pharmacol. 2:189-206, 1947; and Arunlakshana, O. and Schild, H. O. Some quantitative uses of drug antagonists. Br. J. Pharmacol. 14:48-58, 1949.

By “binding affinity” is meant the ability of a compound or drug to bind to its biological target, expressed herein as Ki (nM).

By “intrinsic activity” is meant the maximal stimulation of adenylyl cyclase achievable by a compound in a melanocortin receptor cell system. The maximal stimulation achieved by α-MSH or NDP-α-MSH is designated as an intrinsic activity of 1.0 (or 100%) and a compound capable of stimulating half the maximal activity that of α-MSH or NDP-α-MSH is designated as having an intrinsic activity of 0.5 (or 50%). A compound of this invention that under assay conditions described herein has an intrinsic activity of 0.7 (70%) or higher is classified as an agonist, a compound with intrinsic activity between 0.1 (10%) and 0.7 (70%) is classified as a partial agonist, and a compound with intrinsic activity below 0.1 (10%) is classified as inactive or having no intrinsic activity. Compounds with intrinsic activity below 0.1 (10%) were typically further evaluated, as further discussed herein, for antagonist effect.

Clinical Applications. The compounds disclosed herein, and compounds selected by the methods disclosed herein, can be used for both medical applications and animal husbandry or veterinary applications. Typically, the product is used in humans, but may also be used in other mammals. The term “patient” is intended to denote a mammalian individual, and is so used throughout the specification and in the claims. The primary applications of this invention involve human patients, but this invention may be applied to laboratory, farm, zoo, wildlife, pet, sport or other animals.

According to one embodiment of the present invention, compounds may be employed that are MC4-R agonists, partial agonists, antagonists, inverse agonists or functionally inactive, yet with demonstrated efficacy in animal models in modifying energy metabolism and feeding behavior. Thus in one embodiment, the compounds are inactive or have no intrinsic activity with respect to MC4-R, but bind MC4-R with high affinity and, in some instances, selectivity, such that the compounds attenuate the binding of both α-MSH and AgRP, and further have demonstrated efficacy, in animal models, in modifying energy metabolism and feeding behavior.

Formulations. The compounds may be formulated by any means such as those known in the art, including but not limited to tablets, capsules, caplets, suspensions, powders, lyophilized forms and aerosols/aerosolizable forms and may be mixed and formulated with buffers, binders, stabilizers, anti-oxidants and other agents known in the art. The compounds may be administered by any systemic or partially systemic means known in the art, including but not limited to intravenous injection, subcutaneous injection, administration through mucous membranes, oral administration, dermal administration, skin patches, aerosols and the like.

One embodiment of the present invention provides a pharmaceutical composition and method for the use thereof that includes one or more compounds disclosed herein, or alternatively one or more compounds selected by a method disclosed herein, and a pharmaceutically acceptable carrier. The compounds may thus be formulated or compounded into pharmaceutical compositions that include at least one compound of this invention, or a compound selected by a method of this invention, together with one or more pharmaceutically acceptable carriers, including excipients, such as diluents, carriers and the like, and additives, such as stabilizing agents, preservatives, solubilizing agents, buffers and the like, as may be desired. Formulation excipients may include polyvinylpyrrolidone, gelatin, hydroxy cellulose, acacia, polyethylene glycol, mannitol, sodium chloride or sodium citrate. For injection or other liquid administration formulations, water containing at least one or more buffering constituents is suitable, and stabilizing agents, preservatives and solubilizing agents may also be employed. For solid administration formulations, any of a variety of thickening, filler, bulking and carrier additives may be employed, such as starches, sugars, fatty acids and the like. For pharmaceutical formulations, it is also contemplated that any of a variety of measured-release, slow-release or time-release formulations and additives may be employed, such that the dosage may be formulated so as to effect delivery of a compound of this invention over a period of time.

The compounds of this invention, including compounds selected by one or more methods disclosed herein, may be in the form of any pharmaceutically acceptable salt. Acid addition salts of the compounds of this invention are prepared in a suitable solvent from the compound and an excess of an acid, such as hydrochloric, hydrobromic, sulfuric, phosphoric, acetic, trifluoroacetic, maleic, succinic or methanesulfonic acids. The acetate salt form is especially useful. Where the compounds of this invention include an acidic moiety, suitable pharmaceutically acceptable salts may include alkali metal salts, such as sodium or potassium salts, or alkaline earth metal salts, such as calcium or magnesium salts.

The compounds disclosed herein, or alternatively one or more compounds selected by a method disclosed herein, and pharmaceutical compositions comprising such compound or compounds, may be administered by injection, which injection may be intravenous, subcutaneous, intramuscular, intraperitoneal or by any other means known in the art. In general, any route of administration by which the compounds of this invention are introduced across an epidermal layer of cells may be employed. Administration means may include administration through mucous membranes, buccal administration, oral administration, dermal administration, inhalation administration, nasal administration and the like. The dosage for treatment is administration, by any of the foregoing means or any other means known in the art, of an amount sufficient to bring about the desired therapeutic effect.

Therapeutically Effective Amount. In general, the actual quantity of compound administered to a patient will vary between fairly wide ranges depending upon the mode of administration, the formulation used, and the response desired. This may readily be determined by one of ordinary skill in the art through means such as pharmacokinetic studies, plasma half-life studies, dose escalation studies, and the like. The dosage for treatment is administration, by any of the foregoing means or any other means known in the art, of an amount sufficient to bring about the desired therapeutic effect. Thus a therapeutically effective amount includes an amount of a compound or pharmaceutical composition of this invention that is sufficient to induce the desired therapeutic or biological effect.

The compounds disclosed herein, and alternatively compounds selected by a method disclosed herein, are highly active in modifying energy metabolism and feeding behavior. For example, a compound can be administered at 0.01, 0.05, 0.1, 0.5, 1, 5, 10, 50, 100, or 500 μg/kg body weight, depending on specific compound selected, the desired therapeutic response, the route of administration, the formulation and other factors known to those of skill in the art. Conventional dose response studies and other pharmacological means may be employed to determine the optimal dose for a desired effect with a given compound, given formulation and given route of administration.

Combination Therapy and Weight Regulation. It is also possible and contemplated to use compounds of this invention, or compounds selected by a method of this invention, in combination with other drugs or agents for treatment of various weight and feeding-related disorders. Thus any compound or method may be employed in combination with any other agent or drug heretofore employed as a diet aid, or for decreasing food intake and/or body weight.

Drugs that reduce energy intake include, in part, various pharmacological agents, referred to as anorectic drugs, which are used as adjuncts to behavioral therapy in weight reduction programs. Classes of anorectic drugs include, but are not limited to, noradrenergic and serotonergic agents. Noradrenergic medications may be described as those medications generally preserving the anorectic effects of amphetamines but with weaker stimulant activity. The noradrenergic drugs, except phenylpropanolamine, generally act through a centrally mediated pathway in the hypothalamus that causes anorexia. Phenylpropanolamine, a racemic mixture of norephedrine esters, causes a release of norepinephrine throughout the body and stimulates hypothalamic adrenoreceptors to reduce appetite.

Suitable noradrenergic agents include, but are not limited to, diethylpropion such as TENUATE™ (1-propanone, 2-(diethylamino)-1-phenyl-, hydrochloride) commercially available from Merrell; mazindol (or 5-(p-chlorophenyl)-2,5-dihydro-3H-imidazo[2,1-a]isoindol-5-ol) such as SANOREX™ commercially available from Novartis or MAZANOR™ commercially available from Wyeth Ayerst; phenylpropanolamine (or Benzenemethanol, alpha-(1-aminoethyl)-, hydrochloride); phentermine (or Phenol, 3-[[4,5-duhydro-1H-imidazol-2-yl)ethyl](4-methylphenyl)amino], monohydrochloride) such as ADIPEX-P™ commercially available from Lemmon, FASTING commercially available from Smith-Kline Beecham and Ionamin™ commercially available from Medeva; phendimetrazine (or (2S,3S)-3,4-Dimethyl-2-phenylmorpholine L-(+)-tartrate (1.1)) such as METRA™ commercially available from Forest, PLEGINE™ commercially available from Wyeth-Ayerst; PRELU-2™ commercially available from Boehringer Ingelheim, and STATOBEX™ commercially available from Lemmon; phendamine tartrate such as THEPHORIN™ (2,3,4,9-Tetrahydro-2-methyl-9-phenyl-1H-indenol[2,1-c]pyridine L-(+)-tartrate (1:1)) commercially available from Hoffmann-LaRoche; methamphetamine such as DESOXYN™ Tablets ((S)—N, (alpha)-dimethylbenzeneethanamine hydrochloride) commercially available from Abbott; and phendimetrazine tartrate such as BONTRIL™ Slow-Release Capsules (−3,4-Dimethyl-2-phenylmorpholine Tartrate) commercially available from Amarin.

Suitable non-limiting serotonergic agents include sibutramine such as MERIDIA™ capsules (a racemic mixture of the (+) and (−) enantiomers of cyclobutanemethanamine, 1-(4-chlorophenyl)-N,N-dimethyl-(alpha)-(2-methylpropyl)-, hydrochloride, monohydrate) commercially available from Knoll, fenfluramine such as Pondimin™ (Benzeneethanamine, N-ethyl-alpha-methyl-3-(trifluoromethyl)-, hydrochloride) commercially available from Robbins; dexfenfluramine such as Redux™ (Benzeneethanamine, N-ethyl-alpha-methyl-3-(trifluoromethyl)-, hydrochloride) commercially available from Interneuron. Fenfluramine and dexfenfluramine stimulate release of serotonin and inhibit its reuptake. Sibutramine inhibits the reuptake of serotonin, norepinephrine and dopamine, but does not stimulate secretion of serotonin.

Other serotonergic agents useful with the practice of the present invention include, but are not limited to, certain auoretic gene 5HT1 a inhibitors (brain, serotonin) such as carbidopa and benserazide as disclosed by U.S. Pat. No. 6,207,699 which is incorporated herein by reference; and certain neurokinin 1 receptor antagonists and selective serotonin reuptake inhibitors including fluoxetine, fluvoxamine, paroxtine, sertraline and other useful compounds as disclosed by U.S. Pat. No. 6,162,805 which is incorporated herein by reference. Other potential inhibitors that may be employed include 5HT2c agonists.

Other useful compounds for reducing energy intake include, but are not limited to, certain aryl-substituted cyclobutylalkylamines as disclosed by U.S. Pat. No. 6,127,424 which is incorporated herein by reference; certain trifluoromethylthiophenylethylamine derivatives as disclosed by U.S. Pat. No. 4,148,923 which is incorporated herein by reference; certain compounds as disclosed by U.S. Pat. No. 6,207,699 which is incorporated herein by reference; certain kainite or AMPA receptor antagonists as disclosed by U.S. Pat. No. 6,191,117 which is incorporated herein by reference; certain neuropeptide receptor subtype 5 as disclosed by U.S. Pat. No. 6,140,354 which is incorporated herein by reference; and certain alpha-blocking agents as disclosed by U.S. Pat. No. 4,239,763 which is incorporated herein by reference.

Moreover, several peptides and hormones regulate feeding behavior. For example, cholecystokinin and serotonin act to decrease appetite and food intake. Leptin, a hormone produced by fat cells, controls food intake and energy expenditure. In obese persons who are losing weight without medications, a decrease in weight is associated with a decrease in circulating levels of leptin, suggesting its role in weight homeostasis. Obese patients with high leptin levels are thought to have peripheral leptin resistance secondary to the down-regulation of leptin receptors. Non-limiting examples of useful compounds affecting feeding behavior include certain leptin-lipolysis stimulated receptors as disclosed by WO 01/21647 which is incorporated herein by reference; certain phosphodiesterase enzyme inhibitors as disclosed by WO 01/35970 which is incorporated herein by reference; certain compounds having nucleotide sequences of the mahogany gene as disclosed by WO 00/05373 which is incorporated herein by reference; and certain sapogenin compounds as disclosed by U.S. Pat. No. 4,680,289 which is incorporated herein by reference.

Other useful compounds include certain gamma peroxisome proliferator activated receptor (PPAR) agonists as disclosed by WO 01/30343 and U.S. Pat. No. 6,033,656 which are incorporated herein by reference and certain polypeptides such as fibroblast growth factor-10 polypeptides as disclosed by WO 01/18210 which is incorporated herein by reference.

Moreover, monoamine oxidase inhibitors that decrease energy intake or increase energy expenditure are useful with the practice of the present invention. Suitable, but non-limiting examples of monoamine oxidase inhibitors include befloxatone, moclobemide, brofaromine, phenoxathine, esuprone, befol, toloxatone, pirlindol, amiflamine, sercloremine, bazinaprine, lazabemide, milacemide, caroxazone and other certain compounds as disclosed by WO 01/12176 which is incorporated herein by reference.

Certain compounds that increase lipid metabolism are also useful with the practice of the present invention. Such compounds include, but are not limited to, useful evodiamine compounds as disclosed by U.S. Pat. No. 6,214,831 which is incorporated herein by reference.

Nutrient partitioning agents and digestive inhibitors provide another strategy in the treatment of obesity by interfering with the breakdown, digestion or absorption of dietary fat in the gastrointestinal tract. Gastric and pancreatic lipases aid in the digestion of dietary triglycerides by forming them into free fatty acids that are then absorbed in the small intestine. Inhibition of these enzymes leads to inhibition of the digestion of dietary triglycerides. Non-limiting examples include a lipase inhibitor, orlistat, such as XENICAL™ capsules ((S)-2-formylamino-4-methyl-pentanoic acid (S)-1-[[(2S, 3S)-3-hexyl-4-oxo-2-oxetanyl]methyl]-dodecyl ester) commercially available from Roche Laboratories and certain benzoxazinone compounds as described by WO 00/40247 which is incorporated herein by reference.

Agents that increase energy expenditure are also referred to as thermogenic medications. Non-limiting examples of suitable thermogenic medications include xanthines, such as caffeine and theophylline, selective β-3-adrenergic agonists for example certain compounds in U.S. Pat. No. 4,626,549 which is incorporated by reference herein, and α-2-adrenergic and growth hormones compounds as described in U.S. Pat. Nos. 4,937,267 and 5,120,713 which are incorporated by reference herein.

Generally, a total dosage of the above-described obesity control agents or medications, when used in combination with a compound of this invention or a compound selected or administered in a method of this invention can range from 0.1 to 3,000 mg/day, preferably from about 1 to 1,000 mg/day and more preferably from about 1 to 200 mg/day in single or 2-4 divided doses. The exact dose, however, is determined by the attending clinician and is dependent on such factors as the potency of the compound administered, the age, weight, condition and response of the patient. Assays and Animal Models.

Selected compounds were tested in assays to determine binding and functional status, and were tested in animal models of penile erection and feeding behavior, as discussed below. The following assays and animal models were employed, with modifications as discussed in the examples.

Competitive inhibition assay using [1125]-NDP-α-MSH. A competitive inhibition binding assay was conducted using membranes prepared from HEK-293 cells transfected to express hMC3-R, hMC4-R or hMC5-R, and from B-16 mouse melanoma cells (containing MC1-R) using 0.2 to 0.4 nM [I¹²⁵]-NDP-α-MSH (New England Nuclear) in 50 mM HEPES buffer containing 1 mM MgCl₂, 2 mM CaCl₂, and 5 mM KCl, at pH 7.2. In certain experiments, HEK-293 cells transfected to express hMC1-R were also employed. The assay tube also contained a chosen concentration of the test compound of this invention, typically a 1 μM concentration, for determining inhibition of the binding of [¹125]-NDP-α-MSH to its receptor. Non-specific binding was measured by inhibition of binding of [I¹²⁵]-NDP-α-MSH in the assay with the presence of 1 μM α-MSH.

The assay mixture was incubated for 90 minutes at room temperature, then filtered and the membranes washed three times with ice cold buffer. The filter was dried and counted in a gamma counter for remaining radioactivity bound to the membranes. 100% specific binding was defined as the difference in radioactivity (cpm) bound to cell membranes in the absence and presence of 1 μM α-MSH. The cpm obtained in presence of test compounds were normalized with respect to 100% specific binding to determine the percent inhibition of [125]-NDP-α-MSH binding. Each assay was conducted in triplicate and the actual mean values are described, with results less than 0% reported as 0%.

Competitive binding assay using [I¹²⁵]-AgRP (83-132). Competitive binding studies using [I¹²⁵]-AgRP (83-132) were carried out on membranes isolated from cells expressing hMC4-R. The assay was performed in 96 well GF/B Millipore multiscreen filtration plates (MAFB NOB10) pre-coated with 0.5% bovine serum albumin (Fraction V). The assay mixture contained 25 mM HEPES buffer (pH 7.5) with 100 mM NaCl, 2 mM CaCl₂, 2 mM MgCl₂, 0.3 mM 1,10-phenanthroline, 0.5% bovine serum albumin, cell membranes, radioligand [I¹²⁵]-AgRP (83-132) (NEN), and increasing concentrations of compounds in a total volume of 200 μL. Binding was measured at radioligand concentrations of 0.2 nM. After incubating for 1 hour at 37° C., the reaction mixture was filtered and washed with an assay buffer containing 500 mM NaCl. The dried discs were punched out from the plate and counted on a gamma counter. Care was taken to limit the specific binding of the ligand to an amount not exceeding 10% of the counts added to the reaction mixture. Data were analyzed using the Prism Graph-Pad curve fitting software.

General method for EC₅₀ determination in functional activity assay. Functional evaluation of compounds at melanocortin receptors was performed by measuring the accumulation of intracellular cAMP in HEK-293 cells expressing hMC3-R, hMC4-R or hMC5-R, and in B-16 mouse melanoma cells expressing MC1-R. Cells suspended in Earle's Balanced Salt Solution containing 10 mM HEPES (pH 7.5), 5 mM MgCl₂, 1 mM glutamine, 0.1% albumin and 0.6 mM 3-isobutyl-1-methyl-xanthine, a phosphodiesterase inhibitor, were plated in 96 well plates at a density of 0.5×10⁵ cells per well. Cells were incubated with the test compounds in the presence or absence of α-MSH for 1 hour at 37° C. cAMP levels in the cell lysates were measured using an EIA kit (Amersham). Data analysis and EC₅₀ values were determined using nonlinear regression analysis with Prism Graph-Pad software.

Functional status. The agonist/antagonist status with respect to MC1-R, MC-3R, MC4-R and MC5-R of certain compounds of the invention was determined. Antagonistic activity was determined by measuring the inhibition of α-MSH-induced or NDP-α-MSH-induced cAMP levels following exposure to graded doses of compounds as in the preceding descriptions.

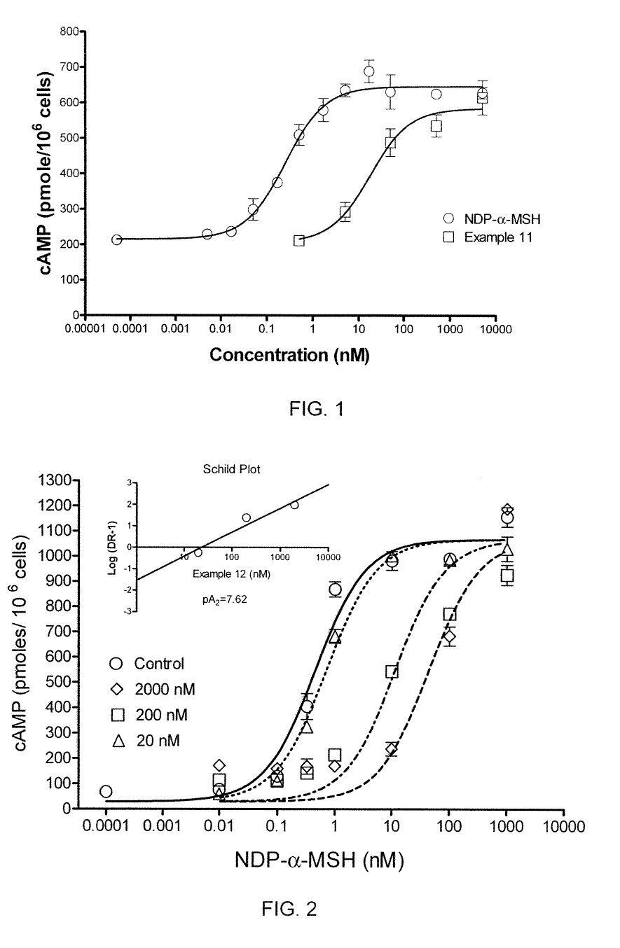

Assay for agonist. Evaluation of the molecules to elicit a functional response in HEK-293 cells expressing MC4-R was accomplished by measuring the accumulation of intracellular cAMP following treatment. Confluent HEK-293 cells expressing MC4-R were detached by enzyme free cell suspension buffer. Cells were suspended in Earle's Balanced Salt Solution containing 10 mM HEPES (pH 7.5), 1 mM MgCl₂, 1 mM glutamine, 0.5% albumin and 0.3 mM 3-isobutyl-1-methyl-xanthine (IBMX), a phosphodiesterase inhibitor. The cells were plated in 96 well plates at a density of 0.5×10⁵ cells per well and pre-incubated for 30 minutes. The cells were then challenged with the test compounds dissolved in DMSO at a concentration range of 0.05-5000 nM in a total assay volume of 200 μL for 1 hour at 37° C. The concentration of DMSO was always held at 1% in the assay mixture. NDP-α-MSH was used as the reference agonist. At the end of the incubation period the cells were disrupted by the addition of 50 μL of lysis buffer from a cAMP EIA kit (Amersham). Complete rupture of the cells was obtained by pipetting the cells multiple times. cAMP levels in the cell lysates were measured after appropriate dilution using the EIA kit (Amersham) method. Data analysis and EC₅₀ values were determined by using nonlinear regression analysis with Prism Graph-Pad software. Compounds at a concentration of 5000 nM that had a response ratio compared to NDP-α-MSH of 0.7 (70%) and above were classified as full agonists. Compounds with a ratio from 0.1 to 0.7 (10% to 70%) were classified as partial agonists. Compounds with a response ratio of less than 0.1 (10%) were evaluated for antagonistic activity. FIG. 1 illustrates typical results, showing the stimulation of cAMP using the compound of Example 11 in HEK-293/MC4-R cells with comparison to NDP-α-MSH.

Assay for Neutral Antagonist. Compounds with a high affinity for binding to MC4-R membranes but with less potency in terms of cAMP accumulation (EC₅₀>1000 nM) and low response ratio (<0.1) were analyzed for their ability to antagonize the stimulatory effect of the agonist NDP-α-MSH. These studies were carried out in HEK-293 cells expressing MC4-R. Cells were incubated with the compounds in the presence of the agonist NDP-α-MSH and the extent of antagonism was measured by the decrease in intracellular cAMP concentrations. Screening the compounds for antagonist activity was performed at a single concentration of NDP-α-MSH (1.0 nM) over a compound concentration range of 0.5-5000 nM. Studies were extended further for compounds exhibiting strong antagonism to derive the pA₂ value from Schild analysis.

Experimental details were similar to the analysis for agonistic activity described above. Briefly, cells were pre-incubated for 30 minutes with the test compounds at concentrations between 0.5 nM and 5000 nM. The cells were then stimulated with NDP-α-MSH at a concentration of 1 nM for 1 hour. For Schild's analysis, the interactions were studied using at least 3 concentrations of the compounds, separated by a log unit, over a full range of the agonist (0.005-5000 nM). cAMP levels were measured in the cell lysates after appropriate dilution. Nonlinear regression analysis with Prism Graph-Pad software was used for Schild analysis and to obtain EC₅₀ values. pA₂ values were derived from the Schild plot of the data. FIG. 2 shows typical results, here the antagonistic activity of the compound of Example 12 in HEK-293 cells expressing MC4-R. The pA₂ value is derived from the Schild plot.

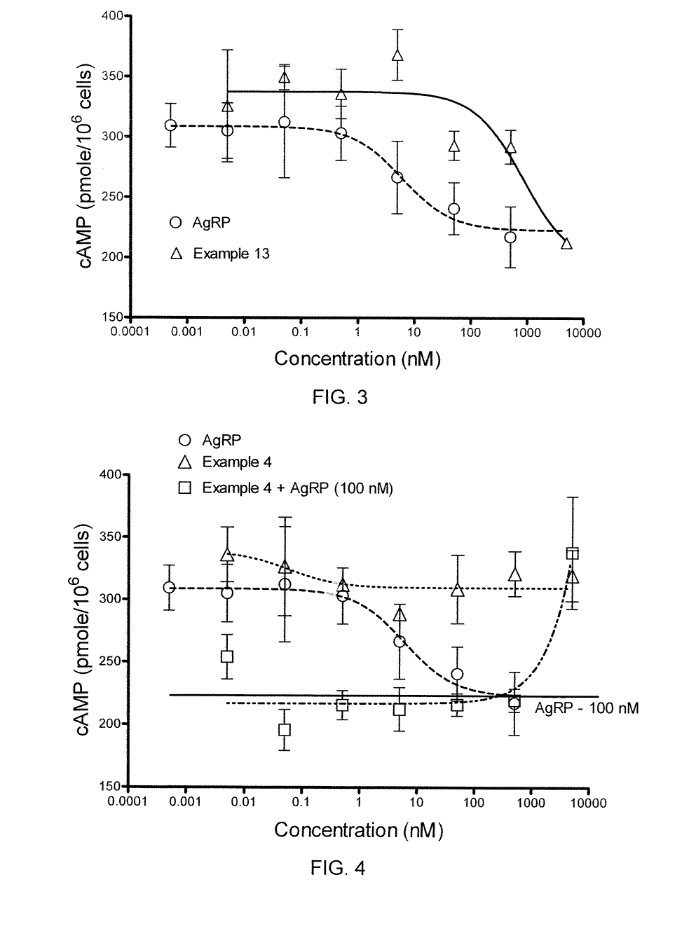

Assay for inverse agonist. Compounds that had a weak EC₅₀ value (EC₅₀>1000 nM) or a low intrinsic activity response ratio (<0.1) were also investigated for their ability to act as inverse agonists, i.e., to decrease the basal or constitutive level of cAMP in HEK-293 cells expressing MC4-R receptors. The experimental protocol was essentially as described above. The cells were exposed to the test compounds over a concentration range of 0.05 nM to 5000 nM for 1 hour at 37° C. AgRP (83-132) was used as the reference inverse agonist. Data analysis and EC₅₀ values were determined by using nonlinear regression analysis with the Prism Graph-Pad software. FIG. 3 shows typical results; here the inverse agonism of the compound of Example 13 in HEK-293 cells expressing MC4-R. Assay for

Antagonists of AgRP. High affinity compounds that displayed no agonistic activity or inverse agonism were investigated for their ability to reverse the antagonistic effects of AgRP, in an effort to identify compounds that compete with AgRP, a recognized inverse agonist. The experimental protocol was essentially as described above for the antagonist assay. Cells were incubated with the compounds in the presence of the inverse agonist AgRP and the extent of reversal of inverse agonism was measured by following the increase in the levels of intracellular cAMP. HEK-293 cells expressing MC4-R were suspended in 10 mM HEPES (pH 7.5) and Earle's Balanced Salt Solution with the phosphodiesterase inhibitor IBMX. The cells were pre-incubated for 30 minutes with the test compounds over a concentration of 0.5 nM to 5000 nM. The cells were then treated with AgRP (100 nM) and incubated for 1 hour. Cells were lysed and cAMP was measured using an EIA kit (Amersham). FIG. 4 shows the antagonistic activity of the compound of Example 4 on AgRP induced inverse agonism in HEK-293 cells expressing MC4-R.

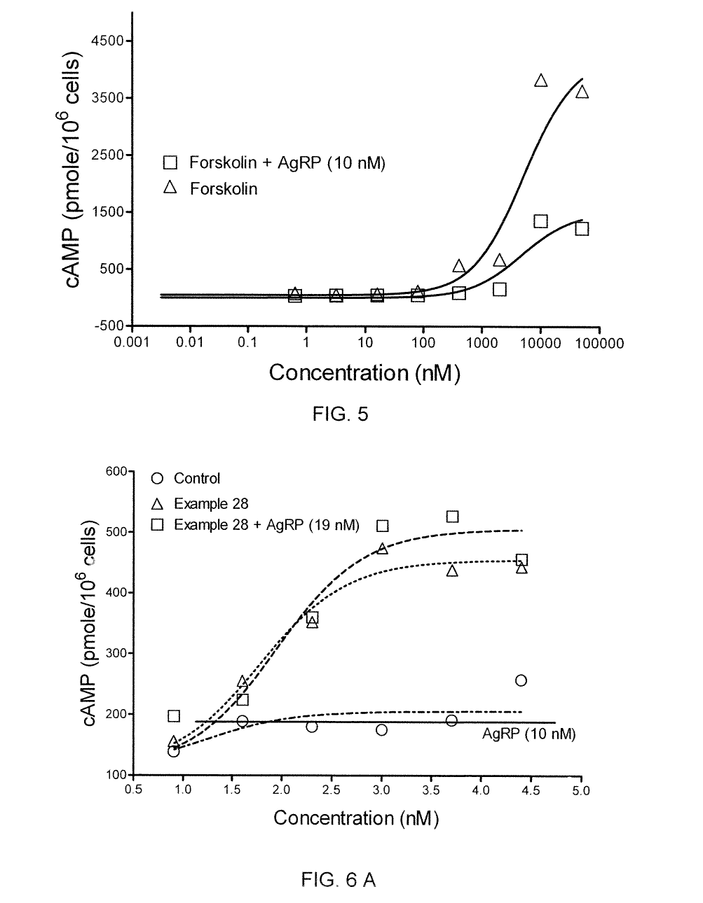

Assay for Antagonists of AgRP in HEK-293/MC4-R Cells stimulated with Forskolin. High affinity compounds that displayed no agonistic activity or inverse agonism were investigated for their ability to reverse the antagonistic effects of AgRP, in an effort to identify compounds that compete with AgRP, a recognized inverse agonist. AgRP causes a decrease in the basal adenylyl cyclase activity only to the extent of 40-50%. Even though the AgRP-induced decrease in cAMP levels is substantial, conventional assay methods, such as those described above, have limited sensitivity, precluding detecting small changes that are reversed by certain antagonists to AgRP. This limitation was circumvented by the use of a non-specific activator, forskolin, which directly activates adenylyl cyclase activity and raises cyclic AMP levels. Forskolin, a cell permeable diterpenoid initially isolated from Coleus forskohlii, has the ability to stimulate adenylyl cyclase activity and increase intracellular cAMP, thus activating cAMP-dependent protein kinase and other cAMP receptor proteins. See, e.g., Seamon, K. B., Daly, J. W. Forskolin: a unique diterpene activator of cyclic AMP-generating system. J. Cyclic Nucleotide Res. 7(4):201-24, 1981; Fradkin, J. E., Cook, G. H., Kilhoffer, M. C., Wolff, J. Forskolin stimulation of thyroid adenylate cyclase and cyclic 3′,5′-adenosine monophosphate accumulation. Endocrinology 111 (3):849-56, 1982; Barber, R., Goka, T. J. Adenylate cyclase activity as a function of forskolin concentration. J. Cyclic Nucleotide Protein Phosphor. Res. 10(1):23-9, 1985; and Laurenza, A., Sutkowski, E. M., Seamon, K. B. Forskolin: a specific stimulator of adenylyl cyclase or a diterpene with multiple sites of action? Trends Pharmacol. Sci. 10(11):442-7, 1989. As illustrated in FIG. 5, it was observed that forskolin stimulated cAMP levels in HEK-293 cells expressing MC4-R can be inhibited by AgRP in a dose dependent fashion. Forskolin elicited a full response in HEK-293 cells expressing MC4-R. The total response was three-fold higher than the response with the strong agonist NDP-α-MSH. The EC₅₀ value was about 5 μM. AgRP inhibited the forskolin-induced response by 65% at a concentration of 10 nM. The difference in forskolin stimulated response and the inhibition due to AgRP in the cAMP levels was exploited to screen compounds for their ability to antagonize AgRP inhibition and decrease levels of cAMP accumulation.

Experimental details for assays included incubating cells with the test compound in the presence of the inverse agonist AgRP with the extent of reversal of the AgRP effect measured by following the increase in the levels of intracellular cAMP. HEK-293 cells expressing MCR-4 were suspended in 10 mM HEPES/Earle's Balanced Salt Solution at pH 7.5 with the phosphodiesterase inhibitor IBMX, and pre-incubated for 30 minutes with the compounds (0.5-5000 nM). The cells were then treated with AgRP (10 nM) and incubated further for 30 minutes. The cells were then treated with forskolin (10 μM) and incubated for an additional 60 minutes. Cells were lysed and cAMP was measured by EIA kit (Amersham).

Expression of recombinant mutant hMC4-R in which a glutamine is exchanged for leucine in amino acid position 250 (L250Q) results in production of increased levels of cAMP in the absence of agonist (Vaisse C. et al., Melanocortin-4 receptor mutations are a frequent and heterogeneous cause of morbid obesity. J Clin Invest 106:253-262, 2000). In some cases, reversal of the inverse agonist activity of AgRP was examined in cells that expressed mutant L250Q MC4-R coupled to a high level of constitutive activity. FIG. 8 illustrates the results obtained with the mutant L250Q MC4-R system.

Penile erection induction. The ability of compounds to induce penile erection (PE) in male rats was evaluated with selected compounds. Male Sprague-Dawley rats weighing 200-250 g were kept on a 12 hour on/off light cycle with food and water ad libitum. All behavioral studies were performed between 10 A.M. and 5 P.M. Groups of 4-8 rats were treated with compounds at a variety of doses via intravenous (IV) or intracerebroventricular (ICV) routes. Immediately after treatment, rats were placed into individual polystyrene cages (27 cm long, 16 cm wide, and 25 cm high) for behavioral observation. Rats were observed for 30 to 60 minutes following IV administration or 120 minutes following ICV administration, and the number of yawns, grooming bouts and PEs were recorded in 10-minute bins. Controls utilized carrier without the test compound. Mean PEs in control groups were 0.17 to 0.5 PEs/rat by IV administration and 0.3 to 0.6 PEs/rat by ICV administration, and thus only PEs with statistically relevant increases over the mean PEs in control groups are reported as inducing PEs.

A PE response in IV animals greater than the mean PEs in control groups but less than 1.0 PEs/rat, particularly with less than all animals responding, was treated as equivocal, and not necessarily distinguishable from vehicle control. In selected instances, compounds with equivocal results in the IV model were tested for penile response in an ICV model, and compounds without a statistically relevant increase over the mean PEs in ICV control groups were determined to not induce PEs.

ICV food intake and body weight change. Change in food intake and body weight was evaluated for selected compounds. Rats with indwelling intracerebroventricular cannulas (ICV rats) were obtained from Hilltop Lab Animals, Inc. (Scottdale, Pa.). Animals were individually housed in conventional plexiglass hanging cages and maintained on a controlled 12 hour on/off light cycle. Water and powdered (LabDiet, 5P00 Prolab RMH 3000) or pelleted (Harlan Teklad 2018 18% Protein Rodent Diet) food was provided ad libitum. For 1 week before treatment, 24-hour food intake and body weight change was recorded to assess a baseline for the group during vehicle treatment. The rats were dosed ICV with vehicle or selected compounds (1-3 nmol). The changes in body weight and food intake for the 24 hour period after dosing were determined. The changes in body weight and food intake for the 48 hour and 72 hour periods after dosing were also measured to determine reversal of changes in body weight and food intake effects back to baseline levels.

IV and IP food intake and body weight change. Change in food intake and body weight was evaluated for selected compounds. Male Sprague-Dawley rats or male C57BLU6 mice were utilized. Animals were individually housed in conventional plexiglass hanging cages and maintained on a controlled 12 hour on/off light cycle. Water and powdered (LabDiet, 5P00 Prolab RMH 3000) or pelleted (Harlan Teklad 2018 18% Protein Rodent Diet) food was provided ad libitum. For 1 week before treatment, 24-hour food intake and body weight change was recorded to assess a baseline for the group during vehicle treatment. The rats were dosed IV with vehicle or selected compounds (0.5-3 mg/kg, and in some cases up to 10 mg/Kg) or dosed IP (intraperitoneally) with vehicle or selected compounds (0.5-10 mg/kg, and in some cases up to 50 mg/kg). The changes in body weight and food intake for the 24 hour period after dosing were determined. The changes in body weight and food intake for the 48 hour and 72 hour periods after dosing were also measured to determined reversal of changes in body weight and food intake effects back to baseline levels.

Determination of mass and nuclear magnetic resonance analysis. The mass values were determined using a Waters MicroMass ZQ device utilizing a positive mode. Mass determinations were compared with calculated values and expressed in the form of mass weight plus one (M+1 or M+H).

Proton NMR data was obtained using a Bruker 300 MHz spectrometer. The spectra were obtained after dissolving compounds in a deuteriated solvent such as chloroform, DMSO, or methanol as appropriate.

Summary of Binding Affinity Data. Table 1 summarizes a comparison of binding affinities of compounds to MC4-R membranes using both iodinated NDP-α-MSH and AgRP as ligands and EC₅₀ values. In Table 1, “Ex.” refers to the example number, “pAg” means partial agonist, “Antag” means antagonist, “NIA” means no intrinsic activity, “ND” means not determined, and pA₂ values are included for selected antagonists.

TABLE 1
Binding affinities and EC₅₀ vales of compounds determined at
MC4-R membranes using [I¹²⁵]-NDP-α-MSH and [I¹²⁵]-AgRP as ligands

| | EC₅₀ | | Ki (nM) | |
|---|---|---|---|---|
| | (nM)/ | | I¹²⁵- | |
| | pA₂ | | NPD-α- | I¹²⁵- |
| Ex. | (M) | Molecular Structure | MSH | AgRP |
| 1 | 296 | NH₂—(CH₂)₆—C(═O)-Ser(Bzl)-D-Phe(4-Cl)-Arg-Trp-NH₂ | 1 | 8 |
| | pAg | | | |
| 2 | 604pAg | 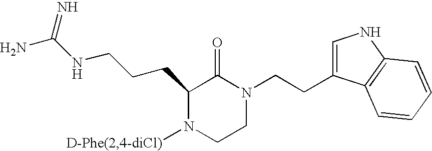 | 15 | 38 |
| 3 | Inactive | 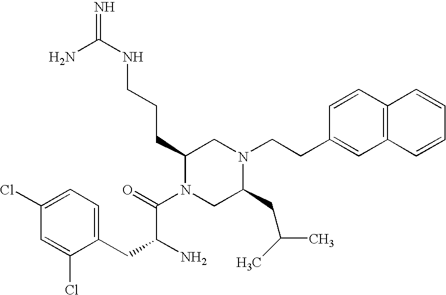 | 48 | 39 |
| 4 | AntagpA₂ =7.69 | 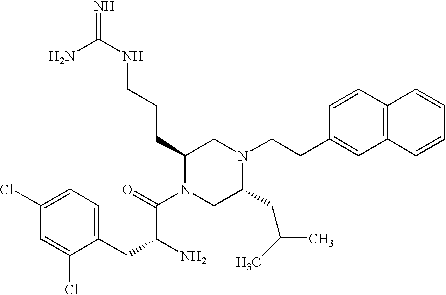 | 5 | 6 |
| 5 | 370pAg | 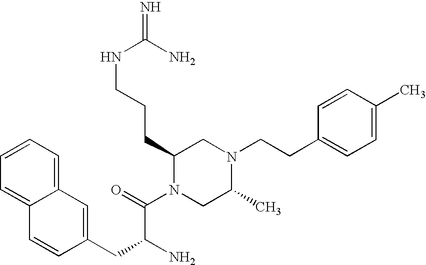 | 29 | 42 |
| 6 | >1000pAg | 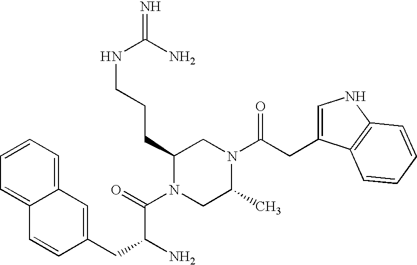 | 35 | 83 |
| 7 | 3800pAg | 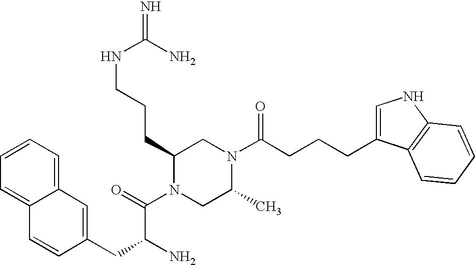 | 90 | 133 |
| 8 | >1000Ag | 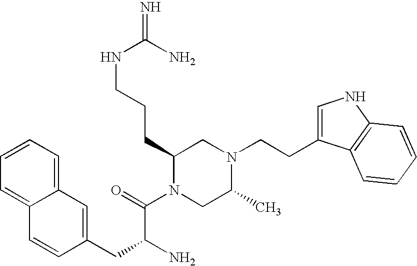 | 37 | 56 |
| 9 | 384Ag | 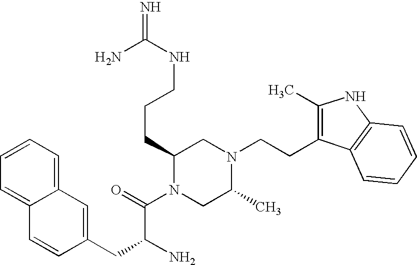 | 26 | 35 |
| 10 | 7pAg | 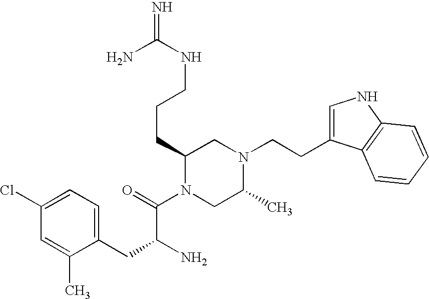 | 5 | 5 |
| 11 | 18Ag | 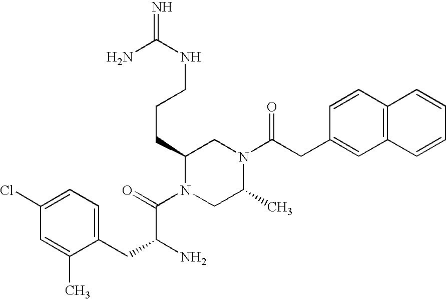 | 3 | 27 |
| 12 | AntagpA₂ =7.75 | 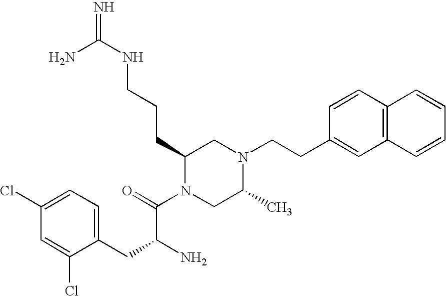 | 2 | 2 |
| 13 | AntagpA₂ =7.28 | 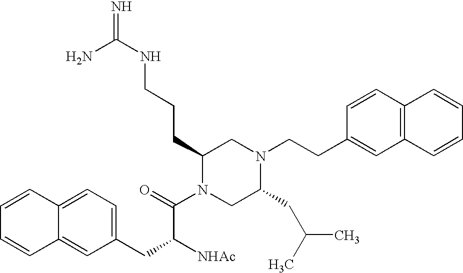 | 6 | 3.7 |
| 14 | 568 | NH₂—(CH₂)₆—C(═O)-Ser(Bzl)-D-Phe(4-Cl)-Arg-Trp-Asp-NH₂ | 9 | ND |
| | pAg | | | |
| 15 | 283 | NH₂—(CH₂)₆—C(═O)-Ser(Bzl)-D-Phe(4-Cl)-Arg-Trp-Asp-Phe-NH₂ | 5 | 0.6 |
| | pAg | | | |
| 16 | >1 μM | NH₂—(CH₂)₆—C(═O)-Ser(Bzl)-D-Phe(4-Cl)-Arg-Trp-Ala-NH₂ | 2 | ND |
| | IA | | | |
| 17 | 17pAg | 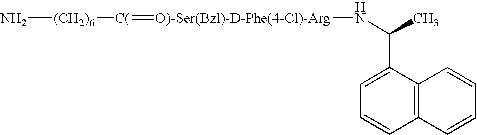 | 7 | 0.6 |
| 18 | 22pAg | 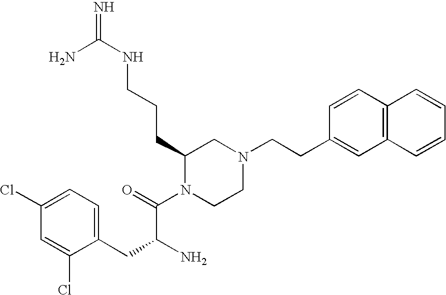 | 3 | 1 |
| 19 | 959pAg | 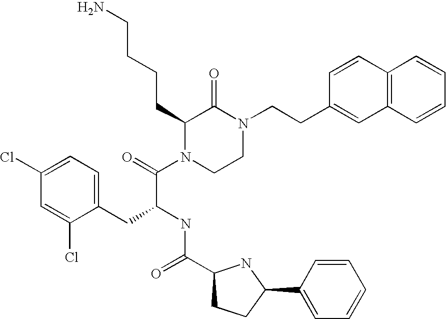 | 50 | 63 |
| 20 | >1000Inactive | 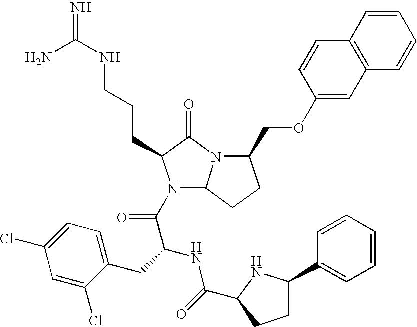 | 12 | 23 |
| 21 | 112pAg | 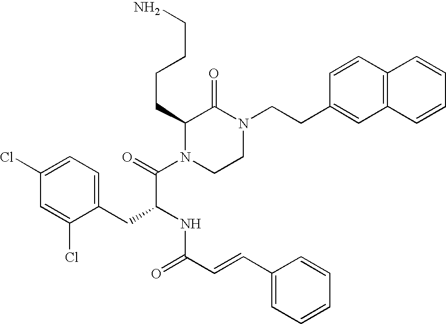 | 6 | 5 |
| 22 | >1000Inactive | 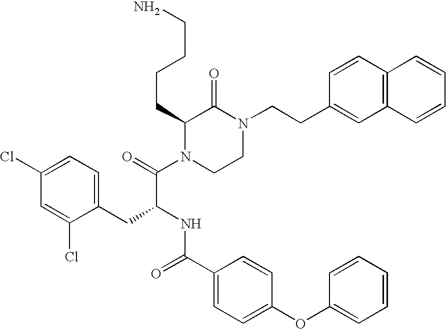 | 1 | 0.9 |
| 23 | AntagpA₂ =8.086 | 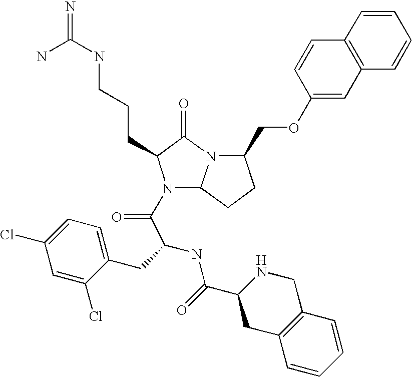 | 1 | 1.2 |
| 24 | >1 μM | Heptanoyl-Thr(Bzl)-D-Phe(4-Cl)-Arg-Trp-NH₂ | 21 | 69 |
| | pAg | | | |
| 25 | >1 μM | 2-naphthylacetyl-1-amino-1-cyclohexane-carbonyl-D- | 40 | 50 |
| | pAg | Phe(4-Cl)-Arg-Trp-NH₂ | | |
| 26 | AntagpA₂ =8.276 | 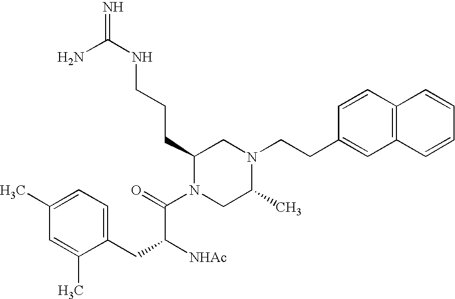 | 1 | 3 |
| 27 | 214pAg | 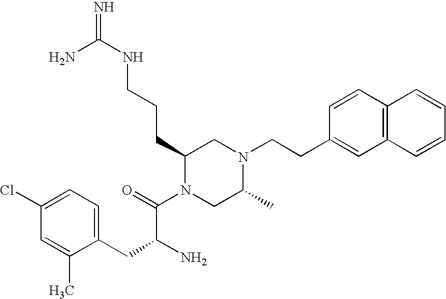 | 29 | ND |
| 28 | 390pAg | 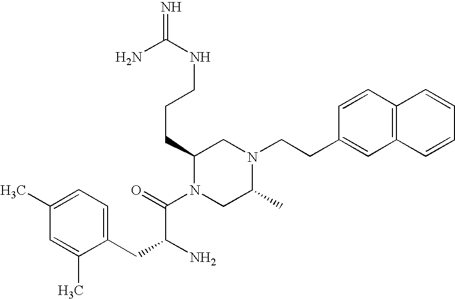 | 11 | 3 |
| 29 | 252Ag | 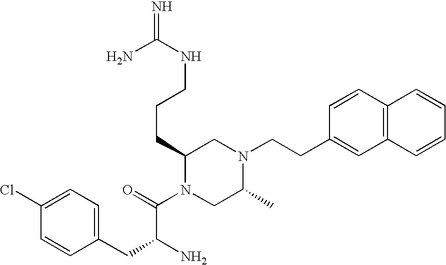 | 11 | 15 |
| 30 | Inactive | 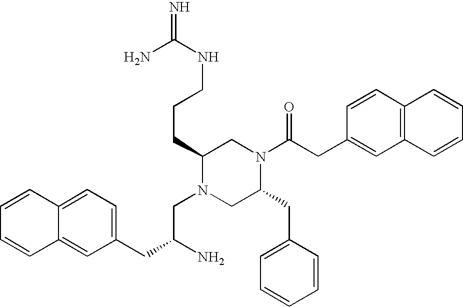 | 55 | 90 |
| 31 | Inactive | 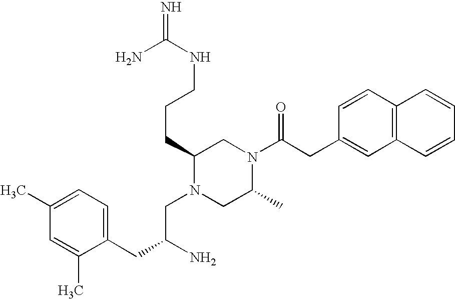 | 90 | 95 |
| 32 | Inactive | 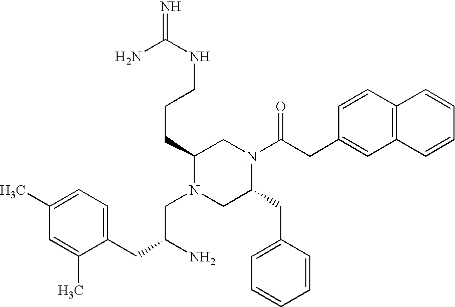 | 8 | 19 |

Applicants have surprisingly and unexpectedly found that melanocortin receptor-specific compounds, which may be either peptide-based or small molecules, may be selected by the methods of this invention which are useful for treatment of obesity and related energy homeostasis diseases, conditions and syndromes, which compounds do not induce a substantial, or any, sexual response in a mammal, such as an erectile response in a male. It has been recognized for several years that MC4-R agonists may be employed for the treatment of obesity and related energy homeostasis diseases, conditions and syndromes. However, a number of melanocortin-receptor specific compounds and agents heretofore evaluated for use in treatment of obesity and related conditions have had unacceptable side effects relating to initiation or induction of a sexual response, including a penile erection response in males. According to one embodiment of the present invention, compounds disclosed herein, and compounds selected by methods of the invention, do not generally induce any sexual response, and do not induce a substantial sexual response.

In a first embodiment, a compound is selected which is an agonist or partial agonist as to MC4-R, but which is further characterized in that (a) the compound attenuates the binding of both α-MSH and AgRP to melanocortin receptors, and specifically MC4-R. In a second embodiment, a compound is selected which is further and additionally characterized in that (b) the compound has a low efficacy, such that the agent is a weak agonist as MC4-R. In a third embodiment, a compound is selected which is further and additionally characterized in that (c) the compound has a Ki (nM) at MC4-R, determined with respect to NDP-α-MSH, that is half or less than half, and preferably substantially less than half, of the EC₅₀ (nM) at MC4-R.

It may thus be seen that the compounds of Examples 1, 2, 5, 6, 7, 8, 9, 10, 11, 15, 17, 18, 19, 21, 24, 25, 28 and 29 may each be classified as partial agonists or agonists as to MC4-R, and yet each meets the requirements of (a) above, in that each of the compounds attenuate the binding of both α-MSH and AgRP to MC4-R. These compounds include peptides, such as those of Examples 1, 15, 24 and 25; modified peptide-based compounds, such as that of Example 17; tri-substituted piperazine compounds such as those of Examples 218, 19 and 21; and tetra-substituted piperazine compounds such as those of Example 5, 6, 7, 8, 9, 10, 11, 28 and 29. It may readily be seen by inspection of Table 1 that the binding affinities of these compounds for α-MSH and AgRP are roughly equivalent, and in many instances are within experimental error not different from each other. Even where the Ki (nM) varies, such as in the peptide of Example 1 which has a Ki (nM) of 1 as to α-MSH binding to MC4-R and of 8 as to AgRP binding to MC4-R, the Ki value nonetheless illustrates high binding affinity with respect to each of α-MSH and AgRP. Similarly, the peptide of Example 1 inhibits, at a 1 μM level, approximately 99% of the binding of α-MSH as to MC4-R, and under similar experimental conditions inhibits approximately 92% of the binding of AgRP, again as to MC4-R.

Among the compounds in the preceding paragraph, a number of the compounds have a low efficacy, and are partial agonists. Thus the compounds of Examples 1, 2, 5, 6, 7, 10, 15, 17, 18, 19, 21, 24 and 28 are classified as partial agonists. In many instances, the partial agonists have very low intrinsic activity; thus the tri-substituted piperazine compound of Example 19 has an intrinsic activity of 0.19, and that of Example 21 has an intrinsic activity of 0.20. The tetra-substituted piperazine compound of Example 28 has an intrinsic activity of 0.16. Thus certain of the compounds are weak partial agonists, with an efficacy of less than about 50%, and in some instances less than about 30%.

Among the compounds described in the first embodiment above, a substantial number have a Ki (nM) at MC4-R, determined with respect to NDP-α-MSH, that is half or less than half, and preferably substantially less than half, of the EC₅₀ (nM) at MC4-R. Thus the compound of Example 1 has a Ki (nM) at MC4-R, determined with respect to NDP-α-MSH, of 1, and an EC₅₀ (nM) at MC4-R of 296. Example 2 has a Ki (nM) at MC4-R, determined with respect to NDP-α-MSH, of 15, and an EC₅₀ (nM) at MC4-R of 604. By examination of Table 1 it may be seen that a number of compounds of this invention have a Ki (nM) that is substantially less than half, and in many instances orders of magnitude less than half, than that of the corresponding EC₅₀ (nM) at MC4-R.

In a fourth embodiment of the invention, Applicants have surprisingly and unexpectedly found that compounds may be selected which may be classified as inactive with respect to agonist or antagonist status, based on the definition of partial agonist given herein, are thus not agonists or partial agonists as to MC4-R, but which are further characterized in that the compounds attenuate the binding of both α-MSH and AgRP to melanocortin receptors, and specifically MC4-R, and which are useful for treatment of obesity and related energy homeostasis diseases, conditions and syndromes, and further do not induce a substantial, or any, sexual response in a mammal, such as an erectile response in a male. Thus the compounds of Examples 3, 20, 22, 30, 31 and 32 are classified as inactive. The compounds of Examples 20 and 22 can further be characterized in that the compound has a Ki (nM) at MC4-R, determined with respect to NDP-α-MSH, that is substantially less than half, of the EC₅₀ (nM) at MC4-R. The compounds thus have some agonist effect, in that an ascertainable EC₅₀ (nM) at MC4-R can be determined, but the intrinsic activity is less than 0.1 (10%), based on maximal stimulation of adenylyl cyclase achievable by the compound in a MC4-R cell system where the maximal stimulation achieved by α-MSH or NDP-α-MSH is designated as an intrinsic activity of 1.0 (or 100%). Thus Example 20 has a Ki (nM) at MC4-R, determined with respect to NDP-α-MSH, of 12, and an EC₅₀ (nM) at MC4-R of >1000. Example 22 has a Ki (nM) at MC4-R, determined with respect to NDP-α-MSH, of 1, and also has an EC₅₀ (nM) at MC4-R of >1000. The compounds of Examples 20 and 22 each caused a decrease in body weight under the described experimental conditions. Other compounds, such as that of Example 16, are similarly inactive with respect to NDP-α-MSH, with the compound of Example 16 having a Ki (nM) at MC4-R of 2, and an EC₅₀ (nM) at MC4-R of >1000, and are effective to cause a decrease in body weight under the described experimental conditions. However, the Ki(nM) of the compound of Example 16 with respect to AgRP was not determined. The compounds of Examples 30, 31 and 32 were each inactive, each had an Ki (nM) at MC4-R determined with respect to both NDP-α-MSH and AgRP that was approximately equal, and each were effective to cause a decrease in body weight under the described experimental conditions.

It is believed that the scientific literature does not provide examples of compounds which are neither agonists nor partial agonists as to MC4-R compared to NDP-α-MSH, but which are nonetheless effective in causing a decrease in body weight. The great majority of the scientific theories heretofore explored have posited that an MC4-R specific compound must at least be a strong partial agonist, and preferably a full agonist, to be effective in causing a decrease in body weight. Applicants have thus surprisingly and unexpectedly found that compounds may bind to MC4-R with high affinity, both with respect α-MSH and optionally also with respect to AgRP, and that are functionally inactive with an intrinsic activity of less than about 10%, and may further have an EC₅₀ (nM) at MC4-R that is substantially higher than the Ki(nM) with respect to α-MSH, and may nonetheless be useful for treatment of obesity and related energy homeostasis diseases, conditions and syndromes. The EC₅₀ (nM) at MC4-R may be at least about one order of magnitude greater than the Ki(nM) with respect to α-MSH, and is preferably at least about two orders of magnitude greater than the Ki(nM) with respect to α-MSH.

In a fifth embodiment of the invention, Applicants have surprisingly and unexpectedly found that compounds may be selected which may be classified as antagonists with respect to MC4-R, and thus by definition are not agonists or partial agonists as to as to MC4-R, but which are further characterized in that the compounds attenuate the binding of both α-MSH and AgRP to melanocortin receptors, and specifically MC4-R, and which are useful for treatment of obesity and related energy homeostasis diseases, conditions and syndromes, and further do not induce a substantial, or any, sexual response in a mammal, such as an erectile response in a male. Thus the compounds of Examples 4, 12, 13, 23 and 26 are classified as antagonists, with pA₂ values as given. The compounds of Examples 4, 12, 13, and 23 each caused a decrease in body weight under the described experimental conditions. It is believed that the scientific and patent literature does not provide examples of compounds which are antagonists as to MC4-R with respect to NDP-α-MSH, or further with an ascertainable and substantial pA₂ value, but which are nonetheless effective in causing a decrease in body weight. The scientific theories heretofore developed have posited that an MC4-R specific compound cannot be an antagonist and be effective in causing a decrease in body weight. Applicants have thus surprisingly and unexpectedly found that compounds may bind to MC4-R with high affinity, both with respect to α-MSH and also with respect to AgRP, and which are functionally antagonists as to MC4-R with respect to NDP-α-MSH, and may nonetheless be useful for treatment of obesity and related energy homeostasis diseases, conditions and syndromes.

In a sixth embodiment of the invention, Applicants have unexpectedly and surprisingly found that a compound may be a full agonist with respect to MC4-R, and further attenuate the binding of both α-MSH and AgRP to MC4-R, and yet have a Ki (nM) at MC4-R, determined with respect to NDP-α-MSH, that is half or less than half, and preferably substantially less than half, that than of the EC₅₀ (nM) at MC4-R. Such compounds can be highly effective for treatment of obesity and related energy homeostasis diseases, conditions and syndromes, yet not induce a sexual response in a mammal, such as an erectile response in a male. Thus, the tetra-piperazine compound of Example 29 is an agonist under the assay conditions described herein, with intrinsic activity of 0.8, and has a Ki (nM) at MC4-R, determined with respect to NDP-α-MSH, of 11, a Ki(nM) with respect of AgRP of 15, and an EC₅₀ (nM) at MC4-R of 252. The compound of Example 29 caused decrease in food intake, yet did not induce penile erections. It is believed that the scientific and patent literature does not provide examples of compounds which are full agonists as to MC4-R with respect to NDP-α-MSH, have approximately equal Ki values with respect to both NDP-α-MSH and AgRP, have an EC₅₀ value that is substantially higher than the corresponding Ki value (here approximately 23 times greater), and yet be effective in causing a decrease in body weight without inducing a sexual response.

It may thus be seen that the invention, as described in the foregoing embodiments and elsewhere in this specification, provides new, novel and useful methods for selecting melanocortin receptor-specific compounds for use in treatment of obesity and related energy homeostasis diseases, conditions and syndromes. Thus, the methods of the invention may be used to screen and select compounds with the desired pharmacological profiles. Heretofore, as described in the Background of the Invention, methods employed by others have simply provided for screening for agonist status, typically as to MC4-R, selecting agonist compounds, and testing such compounds. This invention illustrates that significantly different mechanisms of action are implicated in selection of compounds for the specific indication of obesity and related energy homeostasis diseases, conditions and syndromes, and further selection of compounds which do not cause unintended effects, such as inducing a sexual response.

The invention further describes and characterizes new classes of compounds that may be employed in pharmaceutical compositions for treatment of obesity and related energy homeostasis diseases, conditions and syndromes. In one aspect, these compounds, which may be peptides, peptide-derived, or small molecules, may be agonists or partial agonists as to MC4-R, but which may further be characterized in that the compounds have high affinity, and on approximately the same order, as to MC4-R with respect to both α-MSH and AgRP, and thus attenuate the binding of both α-MSH and AgRP to MC4-R. These compounds may further optionally be characterized in that they have Ki (nM) at MC4-R, determined with respect to NDP-α-MSH, that is half or less than half, and preferably substantially less than half, of the EC₅₀ (nM) at MC4-R. Thus the EC₅₀ (nM) at MC4-R may be 2 times, 5 times, 10 times, 20 times, or more higher than the corresponding Ki (nM) at MC4-R, determined with respect to NDP-α-MSH.

In a second aspect, the invention describes and characterizes a second class of compounds that may be employed in pharmaceutical compositions for treatment of obesity and related energy homeostasis diseases, conditions and syndromes, which compounds, including peptides, peptide-derived, or small molecules, are inactive as to MC4-R, but which may further be characterized in that the compounds have high affinity, and on approximately the same order, to MC4-R with respect to both α-MSH and AgRP, and thus attenuate the binding of both α-MSH and AgRP to MC4-R. These compounds may further optionally be characterized in that they have Ki (nM) at MC4-R, determined with respect to NDP-α-MSH, that is half or less than half, and preferably substantially less than half, of the EC₅₀ (nM) at MC4-R. The compounds are “inactive as to MC4-R” in that the intrinsic activity is less than about 0.1 (10%), based on maximal stimulation of adenylyl cyclase achievable by the compound in a MC4-R cell system where the maximal stimulation achieved by α-MSH or NDP-α-MSH is designated as an intrinsic activity of 1.0 (or 100%). Thus the EC₅₀ (nM) at MC4-R may be 2 times, 5 times, 10 times, 20 times, 100 times or more higher than the corresponding Ki (nM) at MC4-R, determined with respect to NDP-α-MSH.

In a third aspect, the invention describes and characterizes a third class of compounds that may be employed in pharmaceutical compositions for treatment of obesity and related energy homeostasis diseases, conditions and syndromes, which compounds, including peptides, peptide-derived, or small molecules, are antagonists as to MC4-R, but which may further be characterized in that the compounds have high affinity, and on approximately the same order, to MC4-R with respect to both α-MSH and AgRP, and thus attenuate the binding of both α-MSH and AgRP to MC4-R. That MC4-R antagonist compounds exist which are effective in treatment of obesity and related energy homeostasis diseases, conditions and syndromes was not heretofore known.

It is hypothesized, without wishing to be bound by theory, that compounds specific for MC4-R and which attenuate the binding of both α-MSH and AgRP to MC4-R, affect the endogenous melanocortin system in mammals such as to cause an alteration therein resulting in a decrease in food intake or alternatively or additionally an alteration in energy homeostasis, thereby resulting in weight loss, or alternatively preventing weight gain. Thus, compounds selected by the methods of this invention, and compounds disclosed herein, may be employed in pharmaceutical compositions for the treatment of obesity and related energy homeostasis diseases, conditions and syndromes.

It may be seen that the invention provides methods that may readily be adapted for use to screen, select and characterize any melanocortin receptor-specific compound, whether a peptide, peptide derivative, small molecule or otherwise. It may further be seen that the methods may be adapted, by selecting different reactants, reaction conditions, reagents, or the like, and still remain within the scope of this invention. Thus, by way of example, and not limitation, other agonist compounds may be used in place of α-MSH, other inverse agonists may be used in place of the specified AgRP sequence, and the like. It is intended to include all such variations within the invention.

A related application, U.S. patent application Ser. No. ______ entitled Methods for Selection of Melanocortin Receptor-Specific Agents for Treatment of Obesity, is being filed concurrently herewith, Attorney Docket No. 056291-5366, and the specification thereof is incorporated herein by reference. The Methods for Selection of Melanocortin Receptor-Specific Agents for Treatment of Obesity application discloses a method for selecting melanocortin receptor-specific compounds for treatment of obesity and related disorders, and in particular compounds for treatment of obesity and related energy homeostasis or feeding disorders characterized by excess weight gain, but which compounds do not induce a sexual response, including a penile response. In the method, a compound is selected which has no or low potency in terms of cAMP accumulation in very low melanocortin receptor density systems, and in particular MC4-R low density systems, but that has high potency, including potency equal to or greater than NDP-α-MSH, again in terms of cAMP expression, in very high melanocortin receptor density systems, and in particular MC4-R high density systems, pharmaceutical preparations defined thereby, and methods and preparations for treatment of obesity and related energy homeostasis and feed disorders which do not induce a sexual response. By the methods of the Methods for Selection of Melanocortin Receptor-Specific Agents for Treatment of Obesity application it may be seen that certain compounds may be selected that are also selected by the methods disclosed herein. Representative compounds selectable by both the methods of the Methods for Selection of Melanocortin Receptor-Specific Agents for Treatment of Obesity application and this application include the compounds of Examples 10, 11, 27, 28 and 29 herein. Thus, another aspect the invention includes a method of selection of a compound, a compound so selected, and a method of treating obesity, which methods incorporate selection of a compound by any method disclosed herein and by any method disclosed in the Methods for Selection of Melanocortin Receptor-Specific Agents for Treatment of Obesity.

It may be further be seen that the invention provides methods that may readily be adapted for use to screen, select and characterize any melanocortin receptor-specific compound, whether a peptide, peptide derivative, small molecule or otherwise. It may further be seen that the methods may be adapted, by selecting different reactants, reaction conditions, reagents, or the like, and still remain within the scope of this invention. Thus, by way of example, and not limitation, other reference compounds may be used in place of α-MSH or AgRP, such as other known or hereafter development melanocortin agonists or inverse agonists, other cell lines may be employed, and the like. In particular, other means and methods for determining the functional activity of a compound, including but not limited to the means and methods heretofore described, may be employed. In general, once the principles of the invention are understood, one of ordinary skill in the art can determine and develop alternative means and methods of practicing the invention. It is intended to include all such variations within the invention.

While the invention is exemplified in terms of differentiation of the effects of melanocortin receptor-specific agents between effects associated with attenuation of food intake and/or body weight and those effects associated with a sexual response, including a penile erection response in males, it may readily be seen that the methods of the invention may be applied to other effects and responses associated with or resulting from administration of a melanocortin receptor-specific agent. In particular, systemic effects resulting from administration of a melanocortin receptor-specific agent, such as pressor effects resulting in an increase in blood pressure, pica behavior, disruption of normal behavioral satiety sequences and various other systemic effects, are believed to be associated with potent agonist responses derived through central melanocortin agents, and are thus believed to be associated with the mechanisms resulting in a sexual response, and not with the mechanisms associated with attenuation of food intake and/or body weight. Thus in another aspect the invention provides a method for differentiation and selection of melanocortin receptor-specific agents useful for attenuating food intake but which agents do not have, or do not substantially have, a pressor effect or other effects associated with agonist agents that do not attenuate the binding of both an agonist to a melanocortin receptor and an inverse agonist to the same melanocortin receptor.

### EXAMPLE 1 NH₂—(CH₂)₆—C(═O)-Ser(Bzl)-D-Phe(4-Cl)-Arg-Trp-NH₂

The peptide compound NH₂—(CH₂)₆—C(═O)-Ser(Bzl)-D-Phe(4-Cl)-Arg-Trp-NH₂ was synthesized by peptide synthesis methods as disclosed in International Patent Application No. PCT/US02/22196. The molecular weight was determined to be 845. Competitive inhibition testing of the compound yielded the following results:

| | | | |
|---|---|---|---|
| MC1-R | MC3-R | MC4-R | MC5-R |

Inhibition at 1 μM (NDP-α-MSH)

| | | | |
|---|---|---|---|
| 33% | 94% | 99% | 73% |

Ki (nM) (NDP-α-MSH)

| | | | |
|---|---|---|---|
| 351 | 27 | 1 | 208 |

Inhibition at 1 μM (AgRP)
MC4-R
92%
Ki (nM) (AgRP)
MC4-R
8

In a cAMP assay using MC1-R, MC4-R and MC5-R, at 1 μM concentrations the compound of Example 1 was a partial agonist at MC4-R.

The EC₅₀ (nM) at MC4-R was determined to be 296 with intrinsic activity of 0.3.

In mouse model IP feeding studies at 3 mg/kg dose levels, a maximal 26% decrease was observed in food intake for a period of 4 hours. In mouse model IN feeding studies at 10 μg/kg dose levels, a maximal 16% decrease was observed in food intake for a period of 4 hours.

In rat model IV feeding studies at 0.75 mg/kg dose levels, a maximal 37% decrease was observed in food intake for a period of 24 hours.

In rat model penile behavior induction experiments at 1 mg/kg (IV), 1.0 mean penile erections per rat were observed with only not more than 80% of rats in the group responding. However, on ICV administration of doses from 0.01 to 5 nmol, no penile erection response was observed.

### EXAMPLE 2 N-(3-{(S)-1-[(R)-2-Amino-3-(2,4-dichloro-phenyl)-propionyl]-4-[2-(1H-indol-3-yl)-ethyl]-3-oxo-piperazin-2-yl}-propyl)-guanidine

A compound of the following structure:

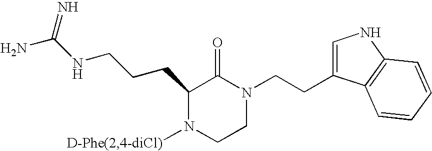

was synthesized by methods described in U.S. patent application Ser. No. 10/762,079. The molecular weight was determined to be 557.5 ESI-MS (M+1). Competitive inhibition testing of the compound yielded the following results (average of triplicates with actual mean values described):

| | | | |
|---|---|---|---|
| MC1-R | MC3-R | MC4-R | MC5-R |

Inhibition at 1 μM (NDP-α-MSH)

| | | | |
|---|---|---|---|
| 7% | 57% | 96% | 35% |

Ki (nM) (NDP-α-MSH)

| | | | |
|---|---|---|---|
| 1409 | 533 | 15 | 1578 |

Inhibition at 1 μM (AgRP)
MC4-R
96%
Ki (nM) (AgRP)
MC4-R
38

In a cAMP assay using MC1-R, MC4-R and MC5-R, at 1 μM concentrations the compound of Example 2 was a partial agonist at MC4-R, MC5-R and MC1-R.

The EC₅₀ (nM) at MC4-R was determined to be 604 with intrinsic activity of 0.3.

In rat model penile behavior induction experiments at 0.3 to 3 μg/kg (IV), the compound did not cause penile erection behavior.

### EXAMPLE 3 N-{3-[1-[2(R)-Amino-3-(2,4-dichloro-phenyl)-propionyl]-5(S)-isobutyl-4-(2-naphthalen-2-yl-ethyl)-piperazin-2(S)-yl]-propyl}-guanidine

The following compound was synthesized by methods described in U.S. patent application Ser. No. 10/837,519, using 2-naphthylacetic acid as J-COOH, L-leucinol as NH₂—CH(R₅)—CH(R₄)—OH, Fmoc-L-Arg(Boc)₂-OH as Prt-NH—CH(R₂)—COOH, and Boc-D-2,4-dichloro-Phe-OH as Q-COOH. It was tested as described above with the results shown. The mass was analyzed as 611.1 (M+H).

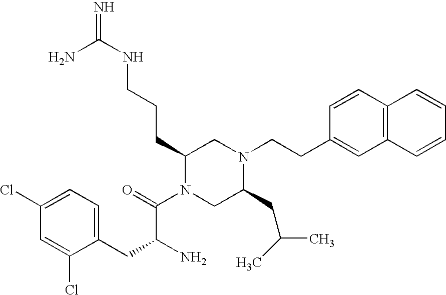

| | | | |
|---|---|---|---|
| MC1-R | MC3-R | MC4-R | MC5-R |

Inhibition at 1 μM (NDP-α-MSH)

| | | | |
|---|---|---|---|
| 4% | 16% | 95% | 28% |

Ki (nM) (NDP-α-MSH)

| | | | |
|---|---|---|---|
| 5895 | 495 | 48 | 698 |

MC4-R
Inhibition at 1 μM (AgRP)
91%
Ki (nM) (AgRP)
39

In a cAMP assay using MC1-R, MC4-R and MC5-R, at 1 μM concentrations the compound of Example 3 exhibited no intrinsic activity (inactive) at MC4-R, and was a partial agonist at MC1-R and MC5-R.

### EXAMPLE 4 N-{3-[1-[2(R)-Amino-3-(2,4-dichloro-phenyl)-propionyl]-5(R)-isobutyl-4-(2-naphthalen-2-yl-ethyl)-piperazin-2(S)-yl]-propyl}-guanidine

The following compound was synthesized by the methods of both Schemes 3 and 5 described in U.S. patent application Ser. No. 10/837,519, using 2-naphthylacetic acid as J-COOH, D-Leucinol as NH₂—CH(R₅)—CH(R₄)—OH, D-leucine methyl ester as NH₂—CH(R₅)—COOCH₃, Fmoc-L-Arg(Boc)₂-OH as Prt-NH—CH(R₂)—COOH, and Boc-D-2,4-dichloro-Phe-OH as Q-COOH. It was tested as described above with the results shown. The mass was analyzed as 611.1 (M+H).

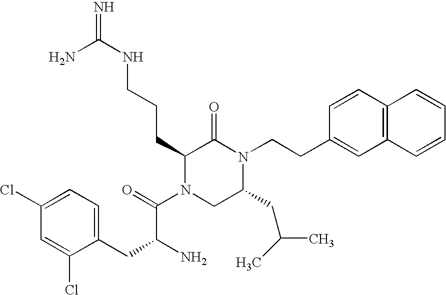

| | | | |
|---|---|---|---|
| MC1-R | MC3-R | MC4-R | MC5-R |

Inhibition at 1 μM (NDP-α-MSH)

| | | | |
|---|---|---|---|
| 21% | 64% | 99% | 75% |

Ki (nM) (NDP-α-MSH)

| | | | |
|---|---|---|---|
| 1364 | 87 | 5 | 160 |

MC4-R
Inhibition at 1 μM (ARP)
100%
Ki (nM) (AgRP)
6

In a cAMP assay using MC1-R, MC4-R and MC5-R, at 1 μM concentrations the compound of Example 4 exhibited no intrinsic activity at MC1-R and MC4-R, and was a partial agonist at MC5-R. The compound was determined to an antagonist as to MC4-R with pA₂ value of 7.7.

In rat model IV and IP feeding studies at 10 mg/kg dose levels, a maximal 89% (IV) and 33% (IP) decrease in food intake was observed for a period of 24 hours. In mouse model IP feeding studies at 10 mg/kg dose levels, a maximal 60% decrease was observed in food intake for a period of 4 hours.

### EXAMPLE 5 N-{3-[1-(2(R)-Amino-3-naphthalen-2-yl-propionyl)-5(R)-methyl-4-(2-p-tolyl-ethyl)-piperazin-2(S)-yl]-propyl}-guanidine

The following compound was synthesized by the method of Scheme 6 in U.S. patent application Ser. No. 10/837,519 using 4-methylphenylacetaldhyde as J-aldehyde, D-alanine methyl ester as NH₂—CH(R₅)—COOCH₃, and Boc-D-2-Nal-OH as Q-COOH. It was tested as described above with the results shown. The mass was analyzed as 515.4 (M+H).

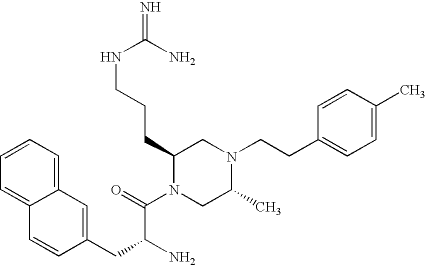

| | | | |
|---|---|---|---|
| MC1-R | MC3-R | MC4-R | MC5-R |

Inhibition at 1 μM (NDP-α-MSH)

| | | | |
|---|---|---|---|
| 32% | 61% | 95% | 65% |

Ki (nM) (NDP-α-MSH)

| | | | |
|---|---|---|---|
| 135 | 164 | 29 | 448 |

MC4-R
Inhibition at 1 μM (AgRP)
93%
Ki (nM) (AgRP)
42

In a cAMP assay using MC1-R, MC3-R, MC4-R and MC5-R, at 1 μM concentrations the compound of Example 5 exhibited no intrinsic activity at MC3-R, and was a partial agonist at MCL-R, MC4-R and MC5-R. An EC₅₀ value of 370 nM on MC-4R was determined with an intrinsic activity of 0.6.

In rat model IV penile erection induction experiments at doses ranging from 0.3 to 30 μg/Kg no penile erection response was observed.

### EXAMPLE 6 N-{3-[1-(2(R)-Amino-3-naphthalen-2-yl-propionyl)-4-(2-1H-indol-3-yl-acetyl)-5(R)-methyl-piperazin-2(S)-yl]-propyl}-guanidine

The following compound was synthesized by the method of Scheme 6 in U.S. patent application Ser. No. 10/837,519 using indole-3-acetic acid as J-COOH, D-alanine methyl ester as NH₂—CH(R₅)—COOCH₃, and Boc-D-2-Nal-OH as Q-COOH. It was tested as described above with the results shown. The mass was analyzed as 554.4 (M+H).

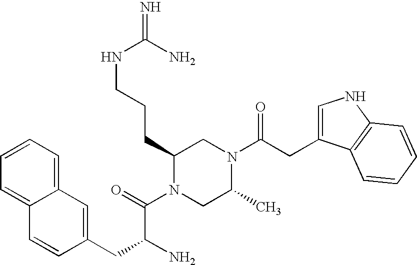

| | | | |
|---|---|---|---|
| MC1-R | MC3-R | MC4-R | MC5-R |

Inhibition at 1 μM (NDP-α-MSH)

| | | | |
|---|---|---|---|
| 20% | 35% | 90% | 6% |

Ki (nM) (NDP-α-MSH)

| | | | |
|---|---|---|---|
| 315 | 1822 | 35 | 1527 |

MC4-R
Inhibition at 1 μM (AgRP)
84%
Ki (nM) (AgRP)
83

In a cAMP assay using MC1-R, MC3-R, MC4-R and MC5-R, at 1 μM concentrations the compound of Example 6 exhibited no intrinsic activity at MC1-R and MC3-R, and was a partial agonist at MC4-R and MC5-R. The EC₅₀ value for MC4-R was over 1000 nM with an intrinsic activity of 0.2.

In rat model IV penile erection induction experiments at doses ranging from 0.3 to 30 μg/Kg no penile erection response was observed.

### EXAMPLE 7 N-{3-[1-(2(R)-Amino-3-naphthalen-2-yl-propionyl)-4-(2-1H-indol-3-yl-butyryl)-5(R)-methyl-piperazin-2(S)-yl]-propyl}-guanidine

The following compound was synthesized by the method of Scheme 6 in U.S. patent application Ser. No. 10/837,519 using indole-3-butyric acid as J-COOH, D-alanine methyl ester as NH₂—CH(R₅)—COOCH₃, and Boc-D-2-Nal-OH as Q-COOH. It was tested as described above with the results shown. The mass was analyzed as 582.6 (M+H).

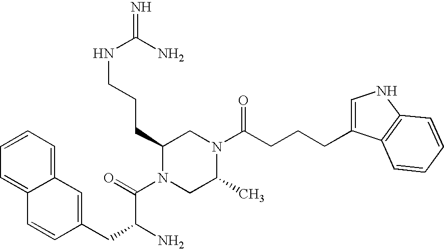

| | | | |
|---|---|---|---|
| MC1-R | MC3-R | MC4-R | MC5-R |

Inhibition at 1 μM (NDP-α-MSH)

| | | | |
|---|---|---|---|
| 0% | 32% | 88% | 62% |

Ki (nM) (NDP-α-MSH)

| | | | |
|---|---|---|---|
| 1203 | 657 | 90 | 271 |

MC4-R
Inhibition at 1 μM (AgRP)
82%
Ki (nM) (AgRP)
133

In a cAMP assay using MC1-R, MC3-R, MC4-R and MC5-R, at 1 μM concentrations the compound of Example 7 exhibited no intrinsic activity at MC1-R, MC3-R and MC5-R, and was a partial agonist at MC4-R. The EC₅₀ value for MC4-R was over 3000 nM with an intrinsic activity of 0.4.

In rat model penile behavior induction experiments by ICV administration of doses from 0.01 to 10 nmol, no penile erection response was observed.

### EXAMPLE 8 N-(3-{1-(2(R)-Amino-3-naphthalen-2-yl-propionyl)-4-[2-(1H-indol-3-yl)-ethyl]-5(R)-methyl-piperazin-2(S)-yl}-propyl)-guanidine

The following compound was synthesized by the method of Scheme 6 in U.S. patent application Ser. No. 10/837,519 using indole-3-acetaldehyde as J-aldehyde, D-alanine methyl ester as NH₂—CH(R₅)—COOCH₃, and Boc-D-2-Nal-OH as Q-COOH. It was tested as described above with the results shown. The mass was analyzed as 540.5 (M+H).

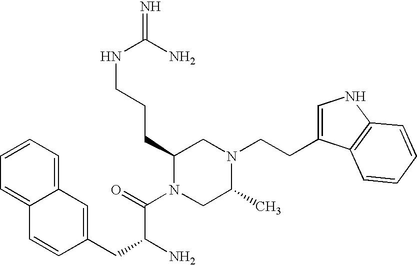

| | | | |
|---|---|---|---|
| MC1-R | MC3-R | MC4-R | MC5-R |

Inhibition at 1 μM (NDP-α-MSH)

| | | | |
|---|---|---|---|
| 2% | 27% | 93% | 51% |

Ki (nM) (NDP-α-MSH)

| | | | |
|---|---|---|---|
| 162 | 2122 | 37 | 808 |

MC4-R
Inhibition at 1 μM (AgRP)
89%
Ki (nM) (AgRP)
56

In a cAMP assay using MC1-R, MC3-R, MC4-R and MC5-R, at 1 μM concentrations the compound of Example 8 exhibited no intrinsic activity at MCL-R, MC3-R and MC5-R, and was a full agonist at MC4-R with EC₅₀ of over 100 nM and an intrinsic activity of 0.9.

### EXAMPLE 9 N-(3-{1-(2(R)-Amino-3-naphthalen-2-yl-propionyl)-5(R)-methyl-4-[2-(2-methyl-1H-indol-3-yl)-ethyl]-piperazin-2(S)-yl}-propyl)-guanidine

The following compound was synthesized by the method of Scheme 6 in U.S. patent application Ser. No. 10/837,519 using 2-methyl-indole-3-acetaldehyde as J-aldehyde, D-alanine methyl ester as NH₂—CH(R₅)—COOCH₃, and Boc-D-2-Nal-OH as Q-COOH. It was tested as described above with the results shown. The mass was analyzed as 554.5 (M+H).

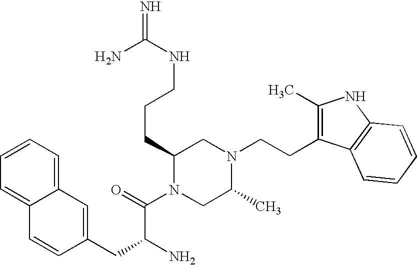

| | | | |
|---|---|---|---|
| MC1-R | MC3-R | MC4-R | MC5-R |

Inhibition at 1 μM (NDP-α-MSH)

| | | | |
|---|---|---|---|
| 73% | 57% | 95% | 76% |

Ki (nM) (NDP-α-MSH)

| | | | |
|---|---|---|---|
| 148 | 451 | 26 | 293 |

MC4-R
Inhibition at 1 μM (AgRP)
92%
Ki (nM) (AgRP)
35

In a cAMP assay using MC1-R, MC3-R, MC4-R and MC5-R, at 1 μM concentrations the compound of Example 9 exhibited no intrinsic activity at MC3-R, was a partial agonist at MC1-R, and was an agonist at MC4-R and MC5-R. The EC₅₀ value for MC4-R was over 384 nM with an intrinsic activity of 0.9.

In rat model penile behavior induction experiments by ICV administration of doses from 0.01 to 10 nM, no penile erection response was observed.

### EXAMPLE 10 N-(3-{1-(2(R)-Amino-(4-chloro-2-methyl-phenyl)-propionyl)-5(R)-methyl-4-[2-(1H-indol-3-yl)-ethyl]-piperazin-2(S)-yl}-propyl)-guanidine

The following compound was synthesized by the method of Scheme 5 in U.S. patent application Ser. No. 10/837,519 using indole-3-acetic acid as J-COOH, D-alanine methyl ester as NH₂—CH(R₅)—COOCH₃, and Boc-D-4-chloro-2-methyl-Phe-OH as Q-COOH. It was tested as described above with the results shown. The mass was analyzed as 538.6 (M+H).

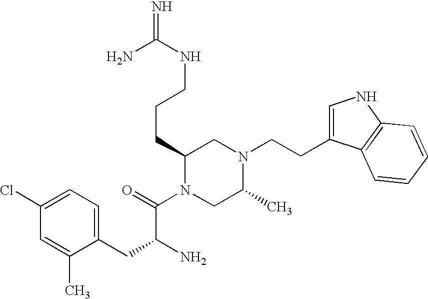

| | | | |
|---|---|---|---|
| MC1-R | MC3-R | MC4-R | MC5-R |

Inhibition at 1 μM (NDP-α-MSH)

| | | | |
|---|---|---|---|
| 40% | 74% | 98% | 75% |

Ki (nM) (NDP-α-MSH)

| | | | |
|---|---|---|---|
| 1730 | 177 | 5 | 360 |

MC4-R
Inhibition at 1 μM (AgRP)
97%
Ki (nM) (AgRP)
5

In a cAMP assay using MC1-R, MC3-R, MC4-R and MC5-R, at 1 μM concentrations the compound of Example 10 exhibited no intrinsic activity at MC1-R, MC3-R and MC5-R, and was a full agonist at MC4-R. The EC₅₀ value for MC4-R was over 7 nM with an intrinsic activity of 1.

In ICV feeding studies at 1 nmol dose levels, a 24 hour decrease in food intake of 16%, and decrease in body weight of 2%, was observed.

In mouse model IP feeding studies at 3 mg/kg dose levels, a maximal 10% decrease was observed in food intake for a period of 4 hours.

In rat model IV and ICV penile erection induction experiments at doses ranging from 0.3 to 30 μg/Kg given IV and at 0.01 to 10 nmole given ICV, no penile erection response was observed.

### EXAMPLE 11 N-{3-[1-[2(R)-amino-3-(4-chloro-2-dimethyl-phenyl)-propionyl]-5(R)-methyl-4-(2-naphthalen-2-yl-acetyl)-piperazin-2(S)-yl]-propyl}-guanidine

The following compound was synthesized by the method of Scheme 7 in U.S. patent application Ser. No. 10/837,519 using 2-naphthyl acetic acid as J-COOH, L-Orn(Boc) methyl ester as NH₂—CH(R₂)—COOCH₃, and Boc-D-4-chloro-2-methyl-Phe-OH as Q-COOH. It was tested as described above with the results shown. The mass was analyzed as 563.4 (M+H).

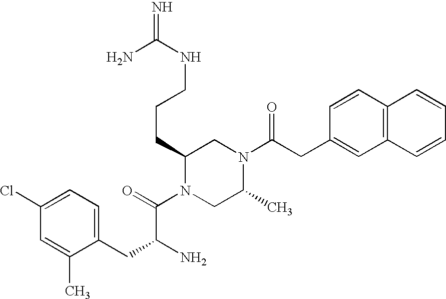

| | | | |
|---|---|---|---|
| MC1-R | MC3-R | MC4-R | MC5-R |

Inhibition at 1 μM (NDP-α-MSH)

| | | | |
|---|---|---|---|
| 3% | 34% | 96% | 36% |

Ki (nM) (NDP-α-MSH)

| | | | |
|---|---|---|---|
| 462 | 398 | 3 | 774 |

MC4-R
Inhibition at 1 μM (AgRP)
89%
Ki (nM) (AgRP)
27

In a cAMP assay for determination of agonist/antagonist status, it was determined that the compound was a partial agonist as to MC1-R and MC3-R, and an agonist as to MC4-R and MC5-R. The EC₅₀ value for MC4-R was 18 nM with an intrinsic activity of 0.9.

In rat model IV feeding studies at 10 mg/kg dose levels, a maximal 33% decrease was observed in food intake for a period of 24 hour.

In mouse model IP feeding studies at 10 mg/kg dose levels, a maximal 27% decrease was observed in food intake for a period of 4 hours.

In rat model IV penile erection induction experiments at doses ranging from 0.3 to 30 μg/kg, 0.5 to 0.7 mean penile erections per rat were observed with not more than 66% animals responding.

### EXAMPLE 12 N-{3-[1-[2(R)-Amino-3-(2,4-dichloro-phenyl)-propionyl]-5(R)-methyl-4-(2-naphthalen-2-yl-ethyl)-piperazin-2(S)-yl]-propyl}-guanidine

The following compound was synthesized by the methods of both Schemes 3 and 5 in U.S. patent application Ser. No. 10/837,519 using 2-naphthylacetic acid as J-COOH, (R)-(−)-2-amino-1-propanol as NH₂—CH(R₅)—CH(R₄)—OH, Fmoc-L-Arg(Boc)₂-OH as Prt-NH—C(R₂)—COOH, D-Alanine methyl ester as NH₂—CH(R₅)—COOCH₃ and Boc-D-2,4-dichloro-Phe-OH as Q-COOH. It was tested as described above with the results shown. The mass was analyzed as 569.3 (M+H).

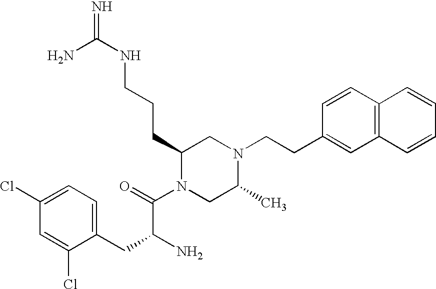

| | | | |
|---|---|---|---|
| MC1-R | MC3-R | MC4-R | MC5-R |

Inhibition at 1 μM (NDP-αMSH)

| | | | |
|---|---|---|---|
| 20% | 72% | 99% | 65% |

Ki (nM) (NDP-α-MSH)

| | | | |
|---|---|---|---|
| 1134 | 95 | 2 | 362 |

MC4-R
Inhibition at 1 μM (AgRP)
99%
Ki (nM) (AgRP)
2

In a cAMP assay for determination of agonist/antagonist status, it was determined that the compound was an antagonist as to MC4-R with pA₂ value of 7.7. At a 1 μM concentration it was inactive at MC1-R, MC3-R and MC5-R.

In rat model IV and IP feeding studies at 10 mg/kg dose levels, a maximal 87% (IV) and 19% (IP) decrease was observed in food intake for a period of 24 hour.

In mouse model IP feeding studies at 3 and 10 mg/kg dose levels, a maximal 10% and 30% decrease, respectively was observed in food intake for a period of 4 hours.

### EXAMPLE 13 N-{2-[2(S)-(3-Guanidino-propyl)-5(R)-isobutyl-4(2-naphthalen-2-yl-ethyl)-piperazin-1-yl]-1(R)-naphthalen-2-ylmethyl-2-oxo-ethyl}-acetamide

The following compound was synthesized by the method of Scheme 5 in U.S. patent application Ser. No. 10/837,519 using 2-naphthylacetic acid as J-COOH, D-leucinol as NH₂—CH(R₅)—CH(R₄)—OH, D-leucine methyl ester as NH₂—CH(R₅)—COOCH₃, and Boc-D-2-Nal-OH as Q-COOH as described in Example 35 thereof. An acetyl group was attached to the amino group of D-2-Nal by reaction of the compound of Example 35 with Ac-OSu in DMF. It was tested as described above with the results shown. The mass was analyzed as 635.9 (M+H).

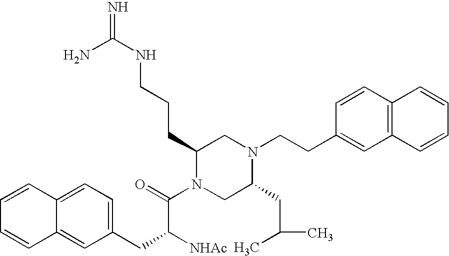

| | | | |
|---|---|---|---|
| MC1-R | MC3-R | MC4-R | MC5-R |

Inhibition at 1 μM (NDP-α-MSH)

| | | | |
|---|---|---|---|
| 0% | 40% | 100% | 64% |

Ki (nM) (NDP-α-MSH)

| | | | |
|---|---|---|---|
| 4616 | 474 | 6 | 378 |

Inhibition at 1 μM (AgRP)

100%

Ki (nM) (AgRP)

3.7

In a cAMP assay using MC1-R, MC3-R, MC4-R and MC5-R, at 1 μM concentrations the compound of Example 13 exhibited no intrinsic activity at MC1-R, MC3-R and MC4-R, and was a partial agonist at MC5-R. At MC4-R it tested as an antagonist with a pA₂ value of 7.3.

In rat model IV and IP feeding studies at 10 mg/kg dose levels, a maximal 65% (IV) and 33% (IP) decrease was observed in food intake for a period of 24 hours.

In mice model IP feeding studies at 10 mg/kg dose levels, a maximal 46% decrease was observed in food intake for a period of 4 hours.

### EXAMPLE 14 NH₂—(CH₂)₆—C(═O)-Ser(Bzl)-D-Phe(4-Cl)-Arg-Trp-Asp-NH₂

The peptide compound NH₂—(CH₂)₆—C(═O)-Ser(Bzl)-D-Phe(4-Cl)-Arg-Trp-Asp-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH)

| | | | |
|---|---|---|---|
| 35% | 73% | 98% | 48% |

Ki (nM) (NDP-α-MSH)

| | | | |
|---|---|---|---|
| 760 | 180 | 9 | 596 |

In a cAMP assay for determination of functionality as to MC4-R, it was determined that the compound had an EC₅₀ (nM) of 568, showed intrinsic activity of 0.5, and was a partial agonist as to MC4-R. It was inactive at MC1-R and MC5-R and a partial agonist at MC3-R.

In ICV feeding studies at 1 nmol dose levels, a 24 hour crease in food intake of 26%, and decrease in body weight of 2%, was observed. In IV feeding studies, a 24 hour decrease in food intake of 6%, and decrease in bodyweight of 1%, was observed.

In rat model IV penile erection induction experiments at 1 μg/kg, no penile erections were observed.

### EXAMPLE 15 NH₂—(CH₂)₆—C(═O)-Ser(Bzl)-D-Phe(4CL)-Arg-Trp-Asp-Phe-NH₂

The peptide compound NH₂—(CH₂)₆—C(═O)-Ser(Bzl)-D-Phe(4-Cl)-Arg-Trp-Asp-Phe-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH)

| | | | |
|---|---|---|---|
| 38% | 66% | 99% | 52% |

Ki (nM) (NDP-α-MSH)

| | | | |
|---|---|---|---|
| 242 | 76 | 5 | 405 |

Inhibition at 1 μM (AgRP)
MC4-R
95%
Ki (nM) (AgRP)
MC4-R
0.6

In a cAMP assay for determination of functionality as to MC4-R, it was determined that the compound was a partial agonist as to MC4-R. The EC₅₀ for MC4-R was 238 nM and showed intrinsic activity of 0.5.

In ICV feeding studies at 1 nmol dose levels, a 24 hour decrease in food intake of 50%, and decrease in body weight of 2%, was observed. In IV feeding studies, a 24 hour decrease in food intake of 30%, and decrease in body weight of 2%, was observed.

In rat model IV penile erection induction experiments at doses ranging from 0.001 to 100 μg/Kg no penile erection response was observed.

### EXAMPLE 16 NH₂—(CH₂)₆—C(═O)-Ser(Bzl)-D-Phe(4-Cl)-Arg-Trp-Ala-NH₂

The peptide compound NH₂—(CH₂)₆—C(═O)-Ser(Bzl)-D-Phe(4-Cl)-Arg-Trp-Ala-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH)

| | | | |
|---|---|---|---|
| 60% | 93% | 100% | 79% |

Ki (nM) (NDP-α-MSH)

| | | | |
|---|---|---|---|
| 142 | 35 | 2 | 112 |

In a cAMP assay for determination of functionality as to MC4-R, it was determined that the compound had an EC₅₀ (nM) of >1 μm and was functionally inactive as to MC4-R.

In ICV feeding studies at 1 nmol dose levels, a 24 hour decrease in food intake of 14%, and decrease in body weight of 2%, was observed. In IV feeding studies, a 24 hour decrease in food intake of 4% was observed.

In rat model IV penile erection induction experiments at a dose of 1 μg/Kg no penile erection response was observed.

### EXAMPLE 17 7′-amino-heptanoyl-Ser(Bzl)-D-Phe(4-Cl)-Arg-S-(−)-1-(1-naphthyl)ethylamide

The following peptide compound was synthesized by standard peptide synthesis methods as described in International Patent Application No. PCT/US02/22196. It was tested as described above with the results shown.

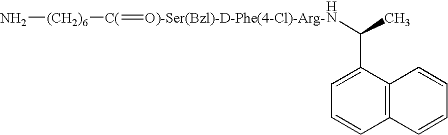

| | | | |
|---|---|---|---|
| MC1-R | MC3-R | MC4-R | MC5-R |

Inhibition at 1 μM (NDP-α-MSH)

| | | | |
|---|---|---|---|
| 46% | 85% | 98% | 64% |

Ki (nM) (NDP-α-MSH)

| | | | |
|---|---|---|---|
| 338 | 125 | 7 | 313 |

Inhibition at 1 μM (AgRP)

100%

Ki (nM) (AgRP)

0.6

In a cAMP assay for determination of functionality as to MC4-R, it was determined that the compound had an EC₅₀ (nM) of 17, showed intrinsic activity of 0.3, and was a partial agonist as to MC4-R.

In ICV feeding studies at 1 nmol dose levels, a 24 hour decrease in food intake of 13%, and decrease in body weight of 1%, was observed.

In rat model IV penile erection induction experiments at a dose of 1 μg/Kg no penile erection response was observed.

### EXAMPLE 18 N-{3-[(S)-1%-[(R)-2-Amino-3-(2,4-dichloro-phenyl)-propionyl]-4-(2-naphthalen-2-yl ethyl)-piperazin-2-yl]-propyl}-guanidine

The following compound was synthesized by methods described in U.S. patent application Ser. No. 10/762,079. It was tested as described above with the results shown.

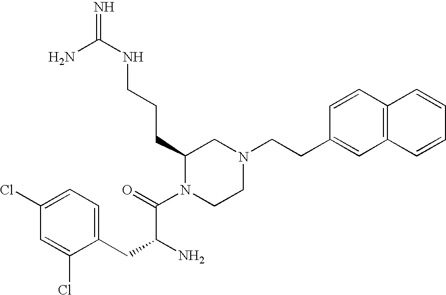

| | | | |
|---|---|---|---|
| MC1-R | MC3-R | MC4-R | MC5-R |

Inhibition at 1 μM (NDP-α-MSH)

| | | | |
|---|---|---|---|
| 6% | 87% | 99% | 75% |

Ki (nM) (NDP-α-MSH)

| | | | |
|---|---|---|---|
| 1198 | 97 | 3 | 259 |

Inhibition at 1 μM (AgRP)

100%

Ki (nM) (AgRP)

1

In a cAMP assay for determination of functionality as to MC4-R, it was determined that the compound had an EC₅₀ (nM) of 22, showed intrinsic activity of 0.4, and was a partial agonist as to MC4-R.

In rat model IV feeding studies at 10 mg/kg dose levels, a maximal 91% decrease was observed in food intake for a period of 24 hours.

In mouse model IP feeding studies at 10 mg/kg dose levels, a maximal 86% decrease was observed in food intake for a period of 4 hours.

In rat model IV penile erection induction experiments at doses ranging from 0.3 to 30 μg/Kg no penile erection response was observed. In rat model ICV penile erection induction experiments at doses ranging from 0.3 to 3 nmole no penile erection response was observed.

### EXAMPLE 19 (2S,5R)-5-Phenyl-pyrrolidine-2-carboxylic acid [(R)-2-[(S)-2-(4-amino-butyl)-4-(2-naphthalen-2-yl-ethyl)-3-oxo-piperazin-1-yl]-1-(2,4-dichloro-benzyl)-2-oxo-ethyl]-amide

The following compound was synthesized by the methods described in U.S. patent application Ser. No. 11/036,282. It was tested as described above with the results shown.

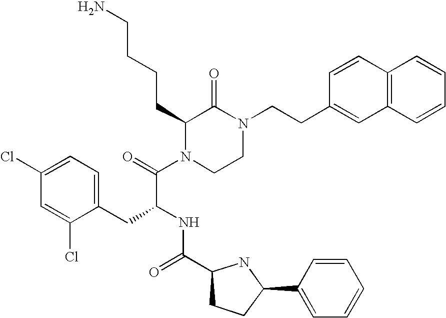

| | | | |
|---|---|---|---|
| MC1-R | MC3-R | MC4-R | MC5-R |

Inhibition at 1 μM (NDP-α-MSH)

| | | | |
|---|---|---|---|
| 0% | 18% | 92% | 51% |

Ki (nM) (NDP-α-MSH)

| | | | |
|---|---|---|---|
| 1370 | 855 | 50 | 789 |

Inhibition at 1 μM (AgRP)

95%

Ki (nM) (AgRP)

63

In a cAMP assay for determination of functionality as to MC4-R, it was determined that the compound had an EC₅₀ (nM) of 959, showed intrinsic activity of 0.2, and was a partial agonist as to MC4-R.

In ICV feeding studies at 1 nmol dose levels, a 24 hour decrease in food intake of 31%, and decrease in body weight of 2%, was observed.

### EXAMPLE 20 (2S,5R)-5-Phenyl-pyrrolidine-2-carboxylic acid {(R)-1-(2,4-dichloro-benzyl)-2-[(2S,5R)-2-(3-guanidino-propyl)-5-(naphthalen-2-yloxymethyl)-3-oxo-hexahydro-pyrrolo[1,2-a]imidazol-1-yl]-2-oxo-ethyl}-amide

The following compound was synthesized by the methods described in U.S. patent application Ser. No. 11/036,282. It was tested as described above with the results shown.

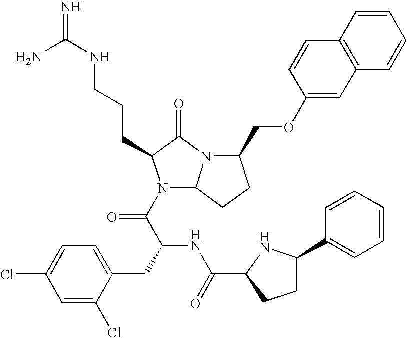

| | | | |
|---|---|---|---|
| MC1-R | MC3-R | MC4-R | MC5-R |

Inhibition at 1 μM (NDP-α-MSH)

| | | | |
|---|---|---|---|
| 16% | 43% | 97% | 86% |

Ki (nM) (NDP-α-MSH)

| | | | |
|---|---|---|---|
| 1041 | 312 | 12 | 99 |

Inhibition at 1 μM (AgRP)

99%

Ki (nM) (AgRP)

23

In a cAMP assay for determination of functionality as to MC4-R, it was determined that the compound had no intrinsic activity.

In ICV feeding studies at 1 nmol dose levels, a 24 hour decrease in food intake of 38.1%, and decrease in body weight of 3%, was observed. In IV feeding studies, a 24 hour decrease in food intake of 1%, and decrease in body weight of 2%, was observed.

In rat model IV penile erection induction experiments at a dose of 1 μg/Kg no penile erection response was observed.

### EXAMPLE 21 (E)-N—[(R)-2-[(S)-2-(4-Amino-butyl)-4-(2-naphthalen-2-yl-ethyl)-3-oxo-piperazin-1-yl]-1-(2,4-dichloro-benzyl)-2-oxo-ethyl]-3-phenyl-acrylamide

The following compound was synthesized by the methods described in U.S. patent application Ser. No. 11/036,282. It was tested as described above with the results shown.

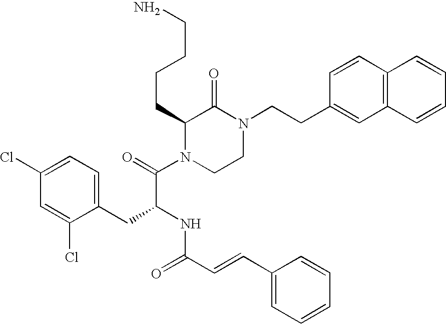

| | | | |
|---|---|---|---|
| MC1-R | MC3-R | MC4-R | MC5-R |

Inhibition at 1 μM (NDP-α-MSH)

| | | | |
|---|---|---|---|
| 30% | 46% | 96% | 60% |

Ki (nM) (NDP-α-MSH)

| | | | |
|---|---|---|---|
| 502 | 648 | 6 | 398 |

Inhibition at 1 μM (AgRP)

100%

Ki (nM) (AgRP)

5

In a cAMP assay for determination of functionality as to MC4-R, it was determined that the compound had an EC₅₀ (nM) of 112, showed intrinsic activity of 0.2, and was a partial agonist as to MC4-R.

In ICV feeding studies at 1 nmol dose levels, a 24 hour decrease in food intake of 4%, and decrease in body weight of 3%, was observed.

In rat model IV penile erection induction experiments at a dose of 1 μg/Kg no penile erection response was observed.

### EXAMPLE 22 N—[(R)-2-[(S)-2-(4-Amino-butyl)-4-(2-naphthalen-2-yl-ethyl)-3-oxo-piperazin-1-yl]-1-(2,4-dichloro-benzyl)-2-oxo-ethyl]-4-phenoxy-benzamide

The following compound was synthesized by the methods described in U.S. patent application Ser. No. 11/036,282. It was tested as described above with the results shown.

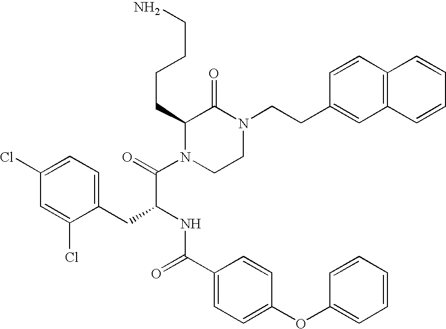

| | | | |
|---|---|---|---|
| MC1-R | MC3-R | MC4-R | MC5-R |

Inhibition at 1 μM (NDP-α-MSH)

| | | | |
|---|---|---|---|
| 35% | 51% | 99% | 51% |

Ki (nM) (NDP-α-MSH)

| | | | |
|---|---|---|---|
| 915 | 150 | 1 | 282 |

Inhibition at 1 μM (AgRP)

100%

Ki (nM) (AgRP)

0.9

In a cAMP assay for determination of functionality as to MC4-R, it was determined that the compound had no intrinsic activity.

In ICV feeding studies at 1 nmol dose levels, a 24 hour decrease in food intake of 2%, and decrease in body weight of 1%, was observed.

In rat model IV penile erection induction experiments at a dose of 1 μg/Kg no penile erection response was observed.

### EXAMPLE 23 (S)-1,2,3,4-Tetrahydro-isoquinoline-3-carboxylic acid {(R)-1-(2,4-dichloro-benzyl)-2-[(2S,5R)-2-(3-guanidino-propyl)-5-(naphthalen-2-yloxymethyl)-3-oxo-hexahydro-pyrrolo[1,2-a]imidazol-1-yl]-2-oxo-ethyl}-amide

The following compound was synthesized by the methods described in U.S. patent application Ser. No. 10/761,889. It was tested as described above with the results shown.

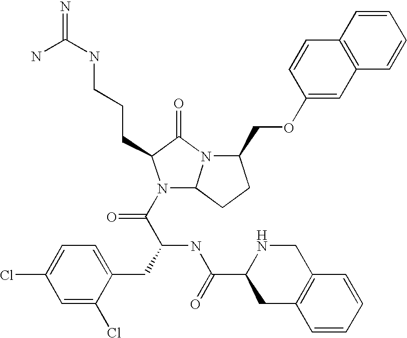

| | | | |
|---|---|---|---|
| MC1-R | MC3-R | MC4-R | MC5-R |

Inhibition at 1 μM (NDP-α-MSH)

| | | | |
|---|---|---|---|
| 75% | 88% | 100% | 96% |

Ki (nM) (NDP-α-MSH)

| | | | |
|---|---|---|---|
| 111 | 42 | 1 | 26 |

Inhibition at 1 μM (AgRP)

100%

Ki (nM) (AgRP)

1.2

In a cAMP assay for determination of functionality as to MC4-R, it was determined that the compound had a pA₂ value of 8.0, and was an antagonist as to MC4-R.

In ICV feeding studies at 1 nmol dose levels, a 24 hour decrease in food intake of 22% was observed.

In rat model IV penile erection induction experiments at a dose of 1 μg/Kg no penile erection response was observed.

### EXAMPLE 24 Heptanoyl-Thr(Bzl)-D-Phe(4-Cl)-Arg-Trp-NH₂

The peptide compound Heptanoyl-Thr(Bzl)-D-Phe(4-Cl)-Arg-Trp-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH)

| | | | |
|---|---|---|---|
| 28% | 39% | 91% | 65% |

Ki (nM) (NDP-α-MSH)

| | | | |
|---|---|---|---|
| 747 | 374 | 21 | 292 |

Inhibition at 1 μM (AgRP)
MC4-R
90%
Ki (nM) (AgRP)
MC4-R
69

In a cAMP assay for determination Of functionality as to MC4-K, it was determined that the compound had an EC₅₀ (nM) of >1000, and was a partial agonist as to MC4-R with an intrinsic activity of 0.4.

In ICV feeding studies at 1 nmol dose levels, a 24 hour decrease in food intake of 18%, and decrease in body weight of 1%, was observed.

In rat model IV penile erection induction experiments at a dose of 1 μg/Kg no penile erection response was observed.

### EXAMPLE 25 2-naphthylacetyl-1-amino-1-cyclohexane-carbonyl-D-Phe(4-Cl)-Arg-Trp-NH₂

The peptide corn pound 2-naphthylacetyl-1-amino-1-cyclohexane-carbonyl-D-Phe(4-Cl)-Arg-Trp-NH₂ was synthesized by standard peptide synthesis methods as described in International Patent Application No. PCT/US02/22196. It was tested as described above with the results shown.

| | | | |
|---|---|---|---|
| MC1-R | MC3-R | MC4-R | MC5-R |

Inhibition at 1 μM (NDP-α-MSH)

| | | | |
|---|---|---|---|
| 25% | 35% | 92% | 63% |

Ki (nM) (NDP-α-MSH)

| | | | |
|---|---|---|---|
| 897 | 453 | 40 | 300 |

Inhibition at 1 μM (AgRP)
MC4-R
91%
Ki (nM) (AgRP)
MC4-R
50

In a cAMP assay for determination of functionality as to MC4-R, it was determined that the compound had an EC₅₀ (nM) of >1000, and was a partial agonist as to MC4-R with an intrinsic activity of 0.2.

In ICV feeding studies at 1 nmol dose levels, a 24 hour decrease in food intake of 21%, and decrease in body weight of 1%, was observed.

### EXAMPLE 26N-{1(R)-(2,4-Dimethyl-benzyl)-2-[2(S)-(3-guanidino-propyl)-5(R)-methyl-4-(2-naphthalen-2-yl-ethyl)-piperazin-1-yl]-2-oxo-ethyl}-acetamide

The following compound was synthesized by the method of Scheme 5 of U.S. patent application Ser. No. 10/837,519, using 2-naphthylacetic acid as J-COOH, D-alaminol as NH₂—CH(R₅)—CH(R₄)—OH, D-alanine methyl ester as NH₂—CH(R₅—COOCH₃, and Boc-D-2,4-dimethyl-Phe-OH as Q-COOH. An acetyl group was attached to the amino group of D-2,4-dimethyl-Phe residue by the method described in Example 36 of U.S. patent application Ser. No. 10/837,519. It was tested as described above with the results shown. The mass was analyzed as 571.9 (M+H).

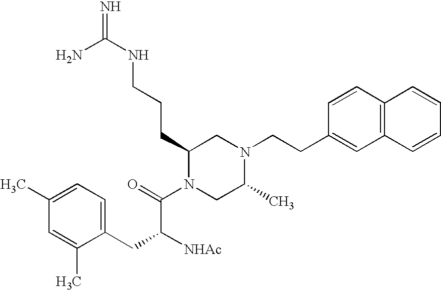

| | | | |
|---|---|---|---|
| MC1-R | MC3-R | MC4-R | MC5-R |

Ki (nM)

| | | | |
|---|---|---|---|
| 568 | 74 | 1 | 43 |

Inhibition at 1 μM (NDP-α-MSH)

| | | | |
|---|---|---|---|
| 28% | 75% | 100% | 89% |

Inhibition at 1 μM (AgRP)

100%

Ki (nM) (AgRP)

0.18

In a cAMP assay using MC4-R, at 1 μM concentrations the compound of Example 26 exhibited no intrinsic activity. It tested as an MC4-R antagonist with a pA₂ value of 8.3.

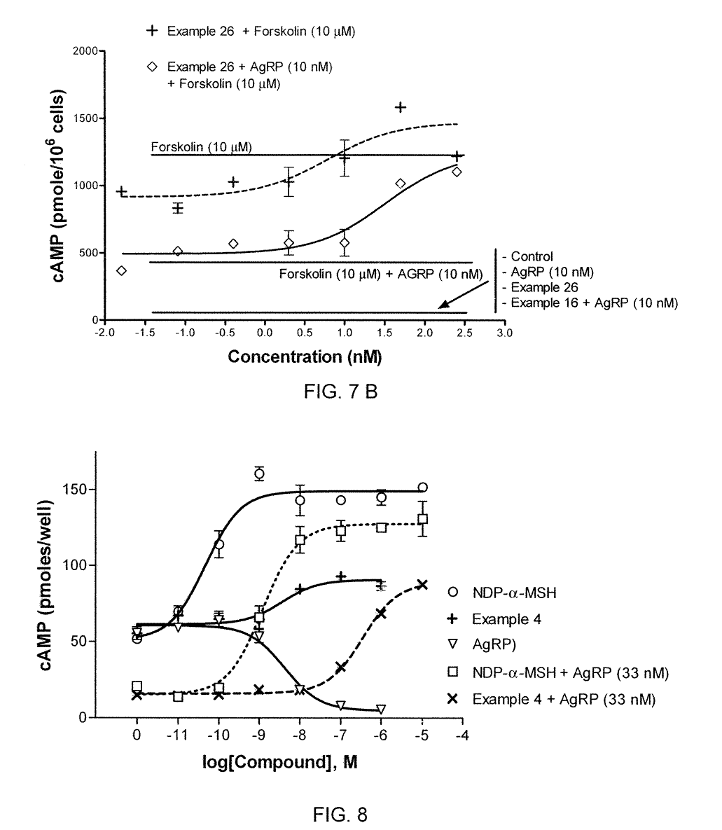

FIGS. 7A and 7B are plots showing the reversal of AgRP inhibition in forskolin-stimulated cAMP levels in HEK-293 cells over-expressing MC4-R receptors by use of the compound of Example 26, with FIG. 7A showing results over the range of 0 to 100 pmole/06 cells of CAMP accumulation, and FIG. 7B showing results over the range of 0 to 2000 pmole/10⁶ cells of cAMP accumulation.

In mouse model IP feeding studies at 10 mg/kg dose levels, a 4 hour decrease in food intake of 34% was observed.

### EXAMPLE 27 N-{3-[1-[2(R)-Amino-3-(2-methyl, 4-chloro-phenyl)-propionyl]-5(R)-methyl-4-(2-naphtha en-2-yl-ethy)-piperazin-2(S)-yl]-propyl}-guanidine

The following compound was synthesized by the methods of U.S. patent application Ser. No. 10/837,519 using 2-naphthylacetic acid as J-COOH, (R)-(−)-2-amino-1-propanol as NH₂—CH(R₅)—CH(R₄)—OH, Fmoc-L-Arg(Boc)₂-OH as Prt-NH—C(R₂)—COOH, D-Alanine methyl ester as NH₂—CH(R₅)—COOCH₃ and Boc-D-2-methyl, 4-chloro-D-Phe-OH as Q-COOH. It was tested as described above with the results shown. The mass was analyzed as 549 (M+H).

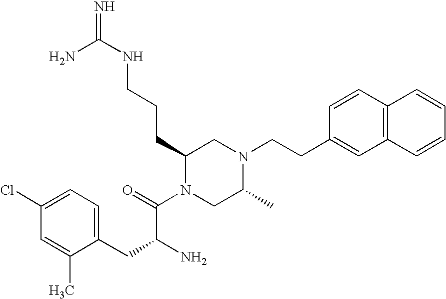

| | | | |
|---|---|---|---|
| MC1-R | MC3-R | MC4-R | MC5-R |

Inhibition at 1 μM (NDP-α-MSH)

| | | | |
|---|---|---|---|
| 6% | 74% | 100% | 77% |

Ki (nM) (NDP-α-MSH)

| | | | |
|---|---|---|---|
| 1052 | 99 | 1 | 219 |

In a cAMP assay for determination of functionality as to MC4-R, it was determined that the compound had an EC₅₀ (nM) of 214, showed intrinsic activity of 0.3, and was a partial agonist as to MC4-R.

In mouse model IP feeding studies at 3 and 10 mg/kg dose levels, a maximum 9% and 19% decrease, respectively, was observed in food intake for a period of 24 hour.

In rat model IV penile erection induction experiments at 1 and 3 mg/kg, no penile erection responses were observed.

### EXAMPLE 28 N-{3-[1-[2(R)-Amino-3-(2,4-dimethyl-phenyl)-propionyl]-5(R)-methyl-4-(2-naphthalen-2-yl-ethyl)-piperazin-2(S)-yl]-propyl}-guanidine

The following compound was synthesized by the methods of U.S. patent application Ser. No. 10/837,519 using 2-naphthylacetic acid as J-COOH, (R)-(−)-2-amino-1-propanol as NH₂—CH(R₅)—CH(R₄)—OH, Fmoc-L-Arg(Boc)₂-OH as Prt-NH—C(R₂)—COOH, D-Alanine methyl ester as NH₂—CH(R₅)—COOCH₃ and Boc-D-2-methyl, 2,4-dimethyl-D-Phe-OH as Q-COOH. It was tested as described above with the results shown. The mass was analyzed as 529 (M+H).

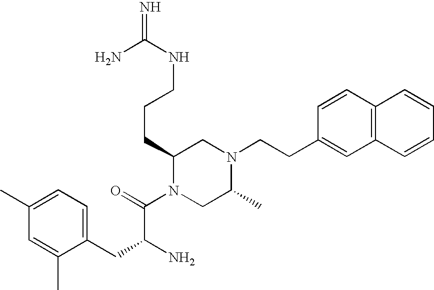

| | | | |
|---|---|---|---|
| MC1-R | MC3-R | MC4-R | MC5-R |

Inhibition at 1 μM (NDP-α-MSH)

| | | | |
|---|---|---|---|
| 9% | 62% | 100% | 59% |

Ki (nM) (NDP-α-MSH)

| | | | |
|---|---|---|---|
| 1233 | 167 | 11 | 665 |

Inhibition at 1 μM (AgRP)

100%

Ki (nM) (AgRP)

3

In a cAMP assay for determination of functionality as to MC4-R, it was determined that the compound had an EC₅₀ (nM) of 321, showed intrinsic activity of 0.2, and was a partial agonist as to MC4-R.

In mouse model IP feeding studies at 3 and 10 mg/kg dose levels, a maximum 11% and 24% decrease, respectively, was observed in food intake for a period of 24 hour.

In rat model IV penile erection induction experiments at 1 and 3 mg/kg, no penile erection were observed.

In rat model ICV penile erection induction experiments at doses of 1 and 3 nmole no penile erection responses were observed.

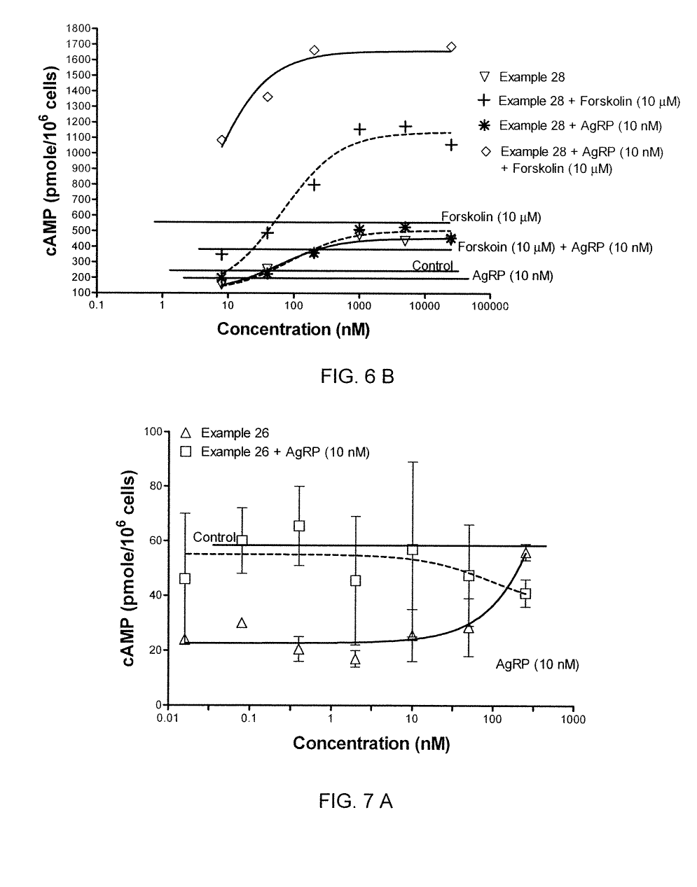

FIGS. 6A and 6B are plots showing the reversal of AgRP inhibition in forskolin-stimulated cAMP levels in HEK-293 cells over-expressing MC4-R receptors by use of the compound of Example 28, with FIG. 6A showing results over the range of 0 to 600 pmole/10⁶ cells of cAMP accumulation, and FIG. 6B showing results over the range of 0 to 2000 pmole/10⁶ cells of cAMP accumulation.

### EXAMPLE 29 N-{3-[1-[2(R)-Amino-3-(4-CHLORO-phenyl)-propionyl]-5(R)-methyl-4-(2-naphthalen-2-yl-ethyl)-piperazin-2(S)-yl]-propyl}-guanidine

The following compound was synthesized by the methods of U.S. patent application Ser. No. 10/837,519 using 2-naphthylacetic acid as J-COOH, (R)-(−)-2-amino-1-propanol as NH₂—CH(R₅)—CH(R₄)—OH, Fmoc-L-Arg(Boc)₂-OH as Prt-NH—C(R₂)—COOH, D-Alanine methyl ester as NH₂—CH(R₅)—COOCH₃ and Boc-D-2-methyl, 4-chloro-D-Phe-OH as Q-COOH. It was tested as described above with the results shown. The mass was analyzed as 535 (M+H).

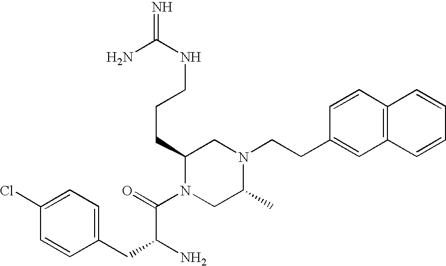

| | | | |
|---|---|---|---|
| MC1-R | MC3-R | MC4-R | MC5-R |

Inhibition at 1 μM (NDP-α-MSH)

| | | | |
|---|---|---|---|
| 64% | 70% | 97% | 61% |

Ki (nM) (NDP-α-MSH)

| | | | |
|---|---|---|---|
| 94 | 160 | 11 | 551 |

Inhibition at 1 μM (AgRP)

97%

Ki (nM) (AgRP)

15

In a cAMP assay for determination of functionality as to MC4-R, it was determined that the compound had an EC₅₀ (nM) of 252, showed intrinsic activity of 0.8, and was an agonist as to MC4-R.

In mouse model IP feeding studies at 3 mg/kg dose level, a maximum 46% decrease was observed in food intake for a period of 4 hours.

In rat model IV penile erection induction experiments at doses ranging 0.003 to 3 mg/kg, no penile erection responses were observed.

### EXAMPLE 30 N-{3-[1-[2(R)-Amino-3-naphthalen-2-yl-propyl]-5(R)-benzyl-4-(2-naphthalen-2-yl-acetyl)-piperazin-2(S)-yl]-propyl}-guanidine

The following compound was synthesized by a modification of the methods of U.S. patent application Ser. No. 10/837,519, and as disclosed in Melanocortin Receptor-Specific Compounds (U.S. patent application Ser. No. 11/464,051; U.S. patent application Ser. No. 11/464,069 and International Patent Application PCT/US06/31474), using 2-naphthylacetic acid as J-COOH, phenylalanine as NH₂—CH(R₅)—CH(R₄)—OH, Fmoc-L-Arg(Boc)₂-OH as Prt-NH—C(R₂)—COOH, and Boc-D-2′-naphthylalanine aldehyde as Q-COOH. It was tested as described above with the results shown. The mass was analyzed as 627 (M+H).

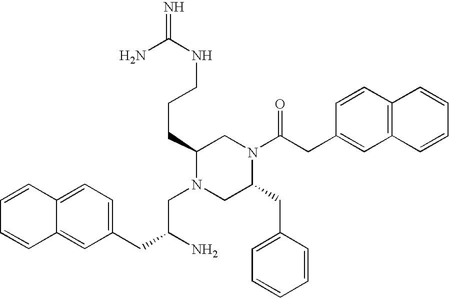

| | | | |
|---|---|---|---|
| MC1-R | MC3-R | MC4-R | MC5-R |

Inhibition at 1 μM (NDP-α-MSH)

| | | | |
|---|---|---|---|
| 32% | 49% | 92% | 87% |

Ki (nM) (NDP-α-MSH)

| | | | |
|---|---|---|---|
| 2774 | 387 | 61 | 178 |

Ki (nM) (AgRP)

90

In a cAMP assay for determination of functionality as to MC4-R, it was determined that the compound was inactive at a 1 μM concentration as to MC4-R.

In mouse model IP feeding studies at 3 and 10 mg/kg dose levels, a maximum 12% and 66% decrease, respectively, was observed in food intake for a period of 4 hours.

### EXAMPLE 31 N-{3-[1-[2(R)-Amino-3-(2,4-dimethyl-phenyl)-propyl]-5(R)-methyl-4-(2-naphthalen-2-yl-acetyl)-piperazin-2(S)-yl]-propyl}-guanidine

The following compound was synthesized by a modification of the methods of U.S. patent application Ser. No. 10/837,519, and as disclosed in Melanocortin Receptor-Specific Compounds (U.S. patent application Ser. No. 11/464,051), using 2-naphthylacetic acid as J-COOH, Fmoc-L-Arg(Boc)₂-OH as Prt-NH—C(R₂)—COOH, D-Alanine methyl ester as NH₂—CH(R₅)—COOCH₃ and Boc-D-2,4 dimethyl-D-Phe-OH aldehyde as Q-COOH. It was tested as described above with the results shown. The mass was analyzed as 528 (M+H).

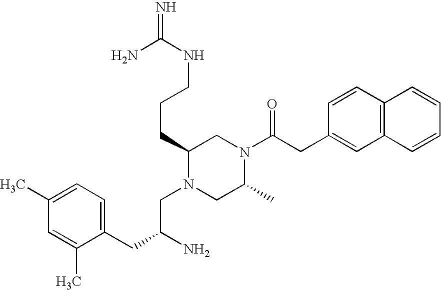

| | | | |
|---|---|---|---|
| MC1-R | MC3-R | MC4-R | MC5-R |

Inhibition at 1 μM (NDP-α-MSH)

| | | | |
|---|---|---|---|
| 11% | 24% | 83% | 83% |

Ki (nM) (NDP-α-MSH)

| | | | |
|---|---|---|---|
| 1355 | 1032 | 106 | 87 |

Ki (nM) (AgRP)

95

In a cAMP assay for determination of functionality as to MC4-R, it was determined that the compound was inactive at a 1 μM concentration as to MC4-R.

In mouse model IP feeding studies at 3 and 10 mg/kg dose levels, a maximum 9% and 66% decrease, respectively, was observed in food intake for a period of 4 hours.

### EXAMPLE 32 N-{3-[1-[2(R)-Amino-3-(2,4-dimethyl-phenyl)-propyl]-5(R)-benzyl-4-(2-naphthalen-2-yl-acetyl)-piperazin-2(S)-yl]-propyl}-guanidine

The following compound was synthesized by a modification of the methods of U.S. patent application Ser. No. 10/837,519, and as disclosed in Melanocortin Receptor-Specific Compounds (U.S. patent application Ser. No. 11/464,051; U.S. patent application Ser. No. 11/464,069 and International Patent Application PCT/US06/31474), using 2-naphthylacetic acid as J-COOH, (R)-(−)-2-amino-1-propanol as NH₂—CH(R₅)—CH(R₄)—OH, Fmoc-L-Arg(Boc)₂-OH as Prt-NH—C(R₂)—COOH, and Boc-D-2-methyl, 4-chloro-D-Phe-OH aldehyde as Q-COOH. It was tested as described above with the results shown. The mass was analyzed as 604 (M+H).

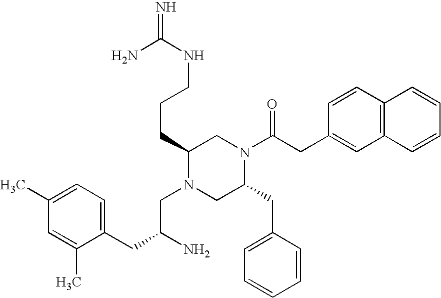

| | | | |
|---|---|---|---|
| MC1-R | MC3-R | MC4-R | MC5-R |

Inhibition at 1 μM (NDP-α-MSH)

| | | | |
|---|---|---|---|
| 0% | 79% | 96% | 94% |

Ki (nM) (NDP-α-MSH)

| | | | |
|---|---|---|---|
| 2882 | 78 | 9 | 115 |

Ki (nM) (AgRP)

19

In a cAMP assay for determination of functionality as to MC4-R, it was determined that the compound was inactive at a 1 μM concentration as to MC4-R.

In mouse model IP feeding studies at 3 and 10 mg/kg dose levels, a maximum 58% and 77% decrease, respectively, was observed in food intake for a period of 4 hours.

The preceding examples can be repeated with similar success by substituting the generically or specifically described reactants and/or operating conditions of this invention for those used in the preceding examples. Thus the agonist selected may be other than α-MSH or NDP-α-MSH, the inverse agonist may be other than AgRP, different assay systems and reporter systems associated with the assay systems may be employed, different reaction conditions may be employed, different sources of melanocortin receptors may be employed, different methods for determining attenuation of binding of an agonist or inverse agonist may be employed, and the like, and all such embodiments are intended to be included within the invention and the claims.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference as if set forth in full.

What is claimed is:

1. A method of treating weight related disorders, the method comprising administering to an obese patient or patient at risk of becoming obese a pharmaceutically effective amount of a compound or a pharmaceutically acceptable salt thereof that attenuates the binding of both an agonist as to one or more melanocortin receptors and an inverse agonist as to the same one or more melanocortin receptors.

2. The method of claim 1 wherein the weight related disorder comprises obesity or energy homeostasis or feeding disorders characterized by excess weight gain.

3. The method of claim 1 wherein the weight related disorder comprises above-optimum weight in a human or non-human mammal.

4. The method of claim 1 wherein the melanocortin receptor is melanocortin-4 receptor
(MC4-R).

5. The method of claim 4 wherein the agonist is alpha-melanocyte stimulating hormone
(α-MSH) and the inverse agonist is agouti-related protein (AgRP).

6. The method of claim 5 wherein α-MSH is NDP-α-MSH and AgRP is AgRP (83-132).

7. The method of claim 4 wherein the compound is an agonist as to human MC4-R.

8. The method of claim 4 wherein the compound is a partial agonist as to human MC4-R.

9. The method of claim 4 wherein the compound is functionally inactive or is a neutral antagonist as to human MC4-R.

10. The method of 4 wherein the compound is an antagonist as to human MC4-R.

11. The method of 4 wherein the compound is an inverse agonist as to human MC4-R.

12. The method of claim 4 wherein the compound is more specific for MC4-R than for any of melanocortin-1 receptor (MC1-R), melanocortin-3 receptor (MC3-R) or melanocortin-5 receptor (MC5-R).

13. The method of claim 12 wherein the compound has a Ki (nM) at MC4-R, determined with respect to α-MSH, which is at least an order of magnitude lower than the Ki (nM) at any of MC1-R, MC3-R or MC5-R.

14. The method of claim 5 wherein the compound attenuates the binding of both α-MSH as to MC4-R and AgRP as to MC4-R by inhibiting, in a competitive inhibition assay, at least about 90% of the binding of each α-MSH and AgRP to MC4-R.

15. The method of claim 6 wherein the compound is an agonist, antagonist, neutral antagonist, or inverse antagonist in a functional MC4-R cAMP assay.

16. The method of claim 4 wherein the compound has an intrinsic activity of less than about 0.1 (10%), based on maximal stimulation of adenylyl cyclase achievable by the compound in a MC4-R cell system where the maximal stimulation achieved by an agonist selected from α-MSH or NDP-α-MSH is designated as an intrinsic activity of 1.0 (100%), and has a Ki (nM) at MC4-R, determined with respect to the agonist, that is half or less than half its EC₅₀ (nM) at MC4-R.

17. The method of claim 16 wherein the Ki (nM) is at least one order of magnitude less than the EC₅₀ (nM).

18. The method of claim 17 wherein the Ki (nM) is more than one order of magnitude less than the EC₅₀ (nM).

19. The method of claim 18 wherein the Ki (nM) is more than two orders of magnitude less than the EC₅₀ (nM).

20. The method of claim 1 wherein the compound is a non-selective MC3-R and MC4-R agonist.

21. The method of claim 1 wherein the compound is a non-selective MC3-R and MC4-R antagonist.

22. The method of claim 1 wherein the compound is an agonist for MC3-R and antagonist for MC4-R.

23. The method of claim 1 wherein the compound is an antagonist for MC3-R and agonist for MC4-R.

24. The method of claim 1 wherein the compound is an inverse agonist for MC3-R and agonist for MC4-R.

25. The method of claim 1 wherein the compound is an inverse agonist for MC3-R and antagonist for MC4-R.

26. The method of claim 1 wherein the compound is an agonist for MC3-R and inverse agonist for MC4-R.

27. The method of claim 1 wherein the compound at least substantially does not induce or initiate a sexual response in a mammal.

28. The method of claim 1 wherein the compound does not induce or initiate a sexual response in the mammal.

29. A method of identifying a compound as a candidate compound for treating weight related disorders, the method comprising the steps of:
providing a melanocortin receptor assay system;
determining if the compound attenuates the binding of an agonist to a melanocortin receptor in the melanocortin receptor assay system;
determining if the compound attenuates the binding of an inverse agonist to the same melanocortin receptor in the melanocortin receptor assay system; and
selecting the compound as a candidate compound If the compound attenuates the binding of both the agonist and the inverse agonist to the melanocortin receptor in the melanocortin receptor assay system.

30. The method of claim 29 wherein the melanocortin receptor assay system comprises an MC4-R assay system.

31. The method of claim 29 wherein the agonist is alpha-melanocyte stimulating hormone (α-MSH) and the inverse agonist is agouti-related protein (AgRP).

32. The method of claim 31 wherein α-MSH is NDP-α-MSH and AgRP is AgRP (83-132).

33. The method of claim 29, further comprising the steps of:
determining the Ki (nM) of the compound with respect to MC4-R and at least one other melanocortin receptor; and
wherein the selection of the compound further comprises selecting the compound as a candidate compound If the compound has a Ki (nM) with respect to MC4-R that is at least about ten times lower than the Ki (nM) with respect to the at least one other melanocortin receptor.

34. The method of claim 33, wherein the at least one other melanocortin receptor comprises MCL-R, MC3-R and MC5-R.

35. The method of claim 29, further comprising the steps of:
determining specificity of the compound for MC1-R, MC3-R, MC4-R and MC5-R; and
wherein the selection of the compound further comprises selecting the compound as a candidate compound If the compound is more specific for MC4-R than for MC1-R, MC3-R or MC5-R.

36. The method of claim 29 wherein selecting the compound further requires that the compound attenuates the binding of both α-MSH as to MC4-R and AgRP as to MC4-R by inhibiting, in a competitive inhibition assay, at least about 90% of the binding of each α-MSH and AgRP to MC4-R.

37. The method of claim 29 wherein compounds having a Ki (nM) of less than about 10 as to both α-MSH binding to MC4-R and AgRP binding to MC4-R are selected.

38. The method of claim 37 wherein compounds having a Ki (nM) of less than about 5 as to both α-MSH binding to MC4-R and AgRP binding to MC4-R are selected.

39. The method of claim 33 wherein compounds having a Ki (nM) as to α-MSH binding to MC4-R that is no more than about five times higher or lower than the Ki (nM) as to AgRP binding to MC4-R are selected.

40. The method of claim 29 further comprising the steps of:
determining the Ki (nM) of the compound at MC4-R;
determining the EC₅₀ of the compound with respect to MC4-R; and
wherein the selection of the compound further requires that the compound has a Ki (nM) value that is half or less than half the EC₅₀ (nM) value.

41. The method of claim 40 wherein the selection of the compound further requires that the compound has a Ki (nM) value that is at least five times less than the EC₅₀ (nM) value.

42. The method of claim 40 wherein the selection of the compound further requires that the compound has a Ki (nM) value that is at least ten times less than the EC₅₀ (nM) value.

43. The method of claim 29, further comprising the steps of:
determining the intrinsic activity of the compound based on maximal stimulation of adenylyl cyclase achievable by the compound in a MC4-R cell system where the maximal stimulation achieved by α-MSH or NDP-α-MSH is designated as an intrinsic activity of 1.0 (100%); and
the selection of the compound further requires that the compound has an intrinsic activity of less than about 0.1 (10%).

44. The method of claim 43, further comprising the steps of:
determining the Ki (nM) of the compound at MC4-R;
determining the EC₅₀ of the compound with respect to MC4-R; and
the selection of the compound further requires that the compound has a Ki (nM) value that is half or less than half the EC₅₀ (nM) value.

45. A method of treating weight related disorders, comprising administration of a pharmaceutically effective amount of a compound that has a Ki(nM) at hMC4-R with respect to both NDP-α-MSH and AgRP of no more than about 50 (nM), and which has an intrinsic activity of less than about 0.3 (30%), based on maximal stimulation of adenylyl cyclase achievable by the compound in a MC4-R cell assay system where the maximal stimulation achieved by α-MSH or NDP-α-MSH is designated as an intrinsic activity of 1.0 (100%).

46. A pharmaceutical composition for treating weight related disorders, which pharmaceutical composition at least substantially does not induce or initiate a sexual response in a mammal, the composition comprising a compound that attenuates the binding of an agonist to a inverse agonist to the same melanocortin receptor in the melanocortin receptor assay system, or a pharmaceutically acceptable salt thereof, and a pharmaceutically acceptable carrier, wherein the composition at least substantially does not induce or initiate a sexual response in a mammal.
